(19) United States
(12) Patent Application Publication
(10) Pub. No.: US 2016/0360987 A1
(43) Pub. Date: Dec. 15, 2016

(54) MAGNETIC FIELD MEASUREMENT APPARATUS AND MAGNETIC FIELD MEASUREMENT METHOD

(71) Applicant: SEIKO EPSON CORPORATION, Tokyo (JP)

(72) Inventors: Mitsutoshi MIYASAKA, Suwa-shi (JP); Toshihiro SAITO, Suwa-shi (JP); Akihiro DEGUCHI, Chino-shi (JP); Ryuji HOKARI, Chino-shi (JP)

(73) Assignee: SEIKO EPSON CORPORATION, Tokyo (JP)

(21) Appl. No.: 15/171,566

(22) Filed: Jun. 2, 2016

(30) Foreign Application Priority Data
Jun. 9, 2015 (JP) 2015-116409

(51) Int. Cl. A61B 5/04; A61B 5/00; G01R 33/035

(52) U.S. Cl. CPC A61B 5/04007; G01R 33/0358; A61B 5/704

(57) ABSTRACT

A magnetic field measurement apparatus includes a magnetic sensor that detects a magnetic field from a subject, a table on which the subject is mounted, a shape measurement device that measures a surface shape of the subject, an average plane calculation unit that calculates an average plane of the surface shape, and a control unit that controls the table so that an opposing surface of the magnetic sensor is parallel to the average plane.

### BACKGROUND

1. Technical Field

The present invention relates to a magnetic field measurement apparatus and a magnetic field measurement method.

2. Related Art

A magnetic field measurement apparatus for measuring a magnetic field of the heart or a magnetic field of the brain, weaker than terrestrial magnetism, has been studied. The magnetic field measurement apparatus is noninvasive, and can measure states of organs without applying a load to a subject. JP-A-2001-170018 discloses a magnetic field measurement apparatus which measures a magnetic field of the heart by using a magnetic detection sensor. According to JP-A-2001-170018, the apparatus includes a table, and a person as a subject is mounted on the table. A superconducting quantum interference element is used in the magnetic detection sensor.

The table can be moved in three directions which are orthogonal to each other. The subject is positioned by using laser light. It is assumed that the three directions which are orthogonal to each other are XYZ directions, and a surface on which the subject is mounted corresponds to an XY plane. First, first laser light is applied obliquely in an XZ plane toward the table, and second laser light is applied obliquely in a YZ plane. In addition, third laser light advancing in the Z direction is applied. The first laser light, the second laser light, and the third laser light intersect each other at a reference point. The reference point is a position which does not move relative to the table.

When the table is moved to a location opposing the magnetic detection sensor, the distance between the magnetic detection sensor and the reference point is a known distance. A subject is mounted on the table. At this time, the reference point is used as a mark, and a position of the subject is measured. The table is moved so that the chest on the heart side of the subject is located at an appropriate location within a measurement range of the magnetic detection sensor. A height of the table is adjusted so that the distance between the magnetic detection sensor and the chest of the subject is an appropriate distance. In this case, a position of the subject can match the magnetic detection sensor with high accuracy by using a measurement value of the distance between the reference point which is clearly shown by the laser light and the chest of the subject.

In the magnetic field measurement apparatus disclosed in JP-A-2001-170018, the table is moved so that a relative position between the superconducting quantum interference element and the subject becomes an appropriate position, but there is a problem in that detection accuracy is low. Therefore, a magnetic field measurement apparatus which can detect a distribution of magnetic vectors of a subject is desirable.

### SUMMARY

An advantage of some aspects of the invention is to solve the problems described above, and the invention can be implemented as the following aspects or application examples.

### Application Example 1

A magnetic field measurement apparatus according to this application example includes a detection unit that detects a magnetic field from a subject; a movable table on which the subject is mounted; a measurement unit that measures a surface shape of the subject; a calculation unit that calculates an average plane of the surface shape; and a control unit that controls the movable table so that an opposing surface to the subject in the detection unit is parallel to the average plane.

According to the present inventor's intensive examination, in the magnetic field measurement apparatus of the related art as disclosed in JP-A-2001-170018, when a subject is mounted on the table, a normal direction of an average plane of the subject differs depending on a body shape of the subject. When a normal direction of an average plane of the chest of the subject is inclined with respect to a direction in which the magnetic detection sensor detects a magnetic vector, an average plane of a surface of the chest and a surface of the magnetic detection sensor on the chest side are not parallel to each other, but intersect. In this case, there may be the occurrence of a location where the distance between the surface of the chest and the magnetic detection unit is short and a location where the distance therebetween is long. A weaker magnetic vector is detected at a location where the distance between the surface of the chest and the magnetic detection sensor is short than a vector at a location where the distance therebetween is long. For this reason, it has been proven in the magnetic field measurement apparatus of the related art that detection accuracy is reduced. In contrast, according to this application example, the magnetic field measurement apparatus includes the movable table, and a subject is mounted on the movable table. The measurement unit measures a surface shape of a measured portion of the subject. Next, the calculation unit calculates an average plane of the surface shape of the subject. The surface shape of the subject is a curved surface, and the calculation unit defines the average plane so that deviation of the measurement unit relative to the surface shape is the minimum. Next, the control unit controls a tilt of the movable table so that the opposing surface is parallel to the average plane. In a state in which the opposing surface of the detection unit is parallel to the average plane, an average distance between the average plane and the opposing surface is shortened, and the detection unit detects a magnetic field coming out of the subject. As a result, the magnetic field measurement apparatus of the application example can detect a distribution of magnetic vectors of the subject with high accuracy without being influenced by a subject's body shape.

As the subject and the opposing surface become more distant from each other, a magnetic field reaching the opposing surface becomes weaker, and thus a signal-to-noise ratio (S/N ratio) of a signal output from the detection unit is lowered. In contrast, if the subject is in contact with the opposing surface, the detection unit receives vibration from the subject, and noise increases due to the vibration. In the application example, the control unit can control a tilt of the movable table so as to make the average plane parallel to the opposing surface of the detection unit, and can cause the subject to sufficiently come close to the opposing surface in a range in which there is no contact therebetween. As a result, the detection unit can detect a magnetic field coming out of the subject with high sensitivity.

### Application Example 2

In the magnetic field measurement apparatus according to the application example, the control unit may control the movable table so that the distance between the opposing surface and the subject becomes a predetermined distance.

According to this application example, the control unit controls the movable table so that the distance between the opposing surface and the subject becomes a predetermined distance. The predetermined distance is longer than the distance which the surface shape of the subject varies through a normal action of the subject such as breathing. The predetermined distance is short within a range in which the subject is not in contact with the detection unit. The control unit controls the movable table so that the distance between the subject and the opposing surface becomes a distance which does not cause contact therebetween due to a normal action of the subject. As a result, the subject can be made to come close to the detection unit within a range in which the subject is not in contact therewith.

### Application Example 3

The magnetic field measurement apparatus according to the application example may further include a magnetic shield unit that encloses the detection unit and the movable table, includes an opening through which the subject comes in and out, and attenuates an entering magnetic field line, and the measurement unit is provided in the opening.

According to this application example, the magnetic field measurement apparatus includes the magnetic shield unit. The magnetic shield unit attenuates an external magnetic field (an entering magnetic field line). The detection unit is provided inside the magnetic shield unit so as to measure a magnetic field. The magnetic shield unit includes the opening, and attenuates an entering magnetic field line. Consequently, the detection unit can perform measurement with less noise. The measurement unit is provided in the opening through which the subject comes in and out. Since the subject passes near the measurement unit, the measurement unit can easily measure a surface shape of the subject.

### Application Example 4

In the magnetic field measurement apparatus according to the application example, the measurement unit may scan the subject with a light beam, first light, and measure a location irradiated with the light beam.

According to this application example, the measurement unit scans the subject with a light beam. A location irradiated with the light beam is measured. A surface shape of the subject has unevenness, and the laser beam is reflected on the unevenness. Therefore, the measurement unit can easily detect the surface shape of the subject by detecting a position of the light beam reflected from the subject.

### Application Example 5

The magnetic field measurement apparatus according to the application example may further include a guide light irradiation unit that applies a light beam for guiding a position where the subject is mounted, and the measurement unit may also be used as the guide light irradiation unit, and the measurement unit may apply a light beam for guiding a position where the subject is mounted. The guide light irradiation unit irradiates a second light for pointing where the subject is mounted.

According to this application example, the measurement unit has a function of a guide light irradiation unit which applies a light beam for guiding a position where the subject is mounted on the movable table, and a function of measuring a surface shape of the subject. The subject is positioned targeting a location indicated by the light beam, and thus the subject can be mounted at a predetermined position on the movable table. The measurement unit also has a function of measuring a surface shape of the subject by scanning the subject with a light beam. Therefore, it is possible to reduce the number of constituent elements of the magnetic field measurement apparatus compared with a case where the magnetic field measurement apparatus separately includes the guide light irradiation unit and the measurement unit. As a result, it is possible to manufacture the magnetic field measurement apparatus with high productivity.

### Application Example 6

In the magnetic field measurement apparatus according to the application example, the movable table may include a plurality of leg portions, and the control unit may control lengths of the leg portions so as to tilt the subject.

According to this application example, the movable table includes a plurality of leg portions. The control unit controls lengths of the leg portions so as to tilt the subject on the movable table. Consequently, the average plane of the subject can be made parallel to the opposing surface. The movable table may have a structure in which a device controlling a tilt is provided at the center thereof. In contrast to this structure, in the application example, loads of the movable table and the subject can be distributed to the plurality of leg portions. Therefore, it is possible to control a tilt of the movable table by using the leg portions with a lightweight structure.

### Application Example 7

In the magnetic field measurement apparatus according to the application example, a portion of the movable table which is moved into the magnetic shield unit may be non-magnetic.

According to this application example, a portion of the movable table which can be moved into the magnetic shield unit is non-magnetic. The non-magnetic portion does not influence measurement of a magnetic field in the detection unit. Therefore, it is possible to prevent magnetization of the movable table from influencing measurement of a magnetic field.

### Application Example 8

In the magnetic field measurement apparatus according to the application example, a location where the detection unit detects a magnetic field may be a surface of the chest opposing the heart.

According to this application example, a location where the detection unit detects a magnetic field is a surface of the chest opposing the heart. A magnetic field generated due to the activity of the heart is output from the surface of the chest. As a result, the detection unit can detect the activity of the heart.

### Application Example 9

A magnetic field measurement method according to this application example includes mounting a subject on a movable table; measuring a surface shape of the subject; calculating an average plane of the subject; tilting the movable table so that an opposing surface to the subject in a detection unit is parallel to the average plane; causing the subject to come close to the opposing surface; and causing the detection unit to detect a magnetic field from the subject.

According to this application example, the subject is mounted on the movable table, and a surface shape of the subject is measured. An average plane of the subject is calculated. The subject is curved, and the average plane is defined so that deviation with the surface shape of the subject is the minimum. Next, a tilt of the movable table is controlled so that an opposing surface of a detection unit is parallel to the average plane of the subject. The subject is made to come close to the opposing surface of the detection unit, and the detection unit detects a magnetic field coming out of the subject.

In this application example, the average plane of the subject is calculated. The control unit controls a tilt of the movable table so that the average plane is parallel to the opposing surface of the detection unit. The subject comes close to the opposing surface without making contact therebetween. Therefore, the subject is made to come close to the opposing surface in a form in which the subject hardly contacts the detection unit. As a result, the detection unit can detect a magnetic field coming out of the subject with high sensitivity.

### Application Example 10

A living body magnetic field measurement apparatus according to this application example includes a magnetic detection unit that detects a distribution of a component in a first direction of a magnetic vector on a first surface of a subject; a table on which the subject is mounted in a state of being in contact with a second surface opposite to the first surface, and that includes a contact surface in contact with the second surface; a measurement unit that measures shapes of the first surface and the second surface; and a control unit that sets a normal direction of the first surface to be the same as the first direction, and controls a shape of the contact surface to be a shape corresponding to the shape of the second surface.

According to this application example, the living body magnetic field measurement apparatus includes the magnetic detection unit, and the magnetic detection unit detects a distribution of a component in a first direction of a magnetic vector on a first surface of a subject. The subject is mounted on a table. The first surface of the subject is directed toward the magnetic detection unit, and the second surface thereof is directed toward the table, and thus the second surface is in contact with the contact surface of the table. The measurement unit measures shapes of the first surface and the second surface. The control unit controls a shape of the contact surface to be a shape corresponding to the shape of the second surface, and sets a normal direction of the first surface of the subject to be the same as the first direction.

Therefore, the normal direction of the first surface of the subject can be adjusted to a direction in which the sensitivity of the magnetic detection unit is high. If the normal direction of the first surface is inclined with respect to the first direction, there may be the occurrence of a location where the distance between the first surface and the magnetic detection unit is short and a location where the distance therebetween is long. The weaker strength of a magnetic vector is detected at the location where the distance between the first surface and the magnetic detection unit is short than a vector at the location where the distance therebetween is long, and thus detection accuracy is reduced. In the application example, the normal direction of the first surface is the same as the first direction. As a result, it is possible to detect the distribution of the magnetic vector of the subject with high accuracy.

### Application Example 11

In the living body magnetic field measurement apparatus according to the application example, the contact surface may be divided into a plurality of division surfaces moved in the first direction, and the number of division surfaces may be 10 or larger and 20 or smaller.

According to this application example, the contact surface is divided into a plurality of division surfaces moved in the first direction. Positions of the plurality of division surfaces in the first direction match the subject, and thus the contact surface can be made to correspond to a shape of the second surface. The number of the plurality of division surfaces is equal to or larger than 10. Therefore, since the ten or more division surfaces are in contact with and support the subject, the subject can be stably supported, and the first surface can be made to be directed in a predetermined direction. The number of the plurality of division surfaces is equal to or smaller than 20. Therefore, the control unit can easily control positions of the division surfaces.

### Application Example 12

In the living body magnetic field measurement apparatus according to the application example, a width of each of the division surfaces may be equal to or more than 5 cm and equal to or less than 15 cm.

According to this application example, a width of each of the division surfaces is equal to or more than 5 cm and equal to or less than 15 cm. Therefore, since the division surfaces are in contact with and support the subject at the interval of 5 cm to 15 cm, the subject can be stably supported, and the first surface can be directed in a predetermined direction.

### Application Example 13

In the living body magnetic field measurement apparatus according to the application example, a movable range in which the division surfaces are moved in the first direction may be equal to or more than 3 cm and equal to or less than 10 cm.

According to this application example, a movable range of the division surfaces is equal to or more than 3 cm and equal to or less than 10 cm. In this case, if the subject is a person, the contact surface can match a shape of the back side of the person. Thus, since the division surfaces are in contact with and support the subject, the subject can be stably supported, and the first surface can be directed in a predetermined direction. Since the movable range is equal to or less than 10 cm, it is possible to easily control the division surfaces.

### Application Example 14

The living body magnetic field measurement apparatus according to the application example may further include a magnetic shield unit that encloses the magnetic detection unit and the table, includes a first opening through which the subject comes in and out, and attenuates an entering magnetic field line, and the control unit is present at a location separated from the first opening.

According to this application example, the living body magnetic field measurement apparatus includes the magnetic shield unit. The magnetic shield unit attenuates an entering magnetic field line. The magnetic detection unit and the table are provided inside the magnetic shield unit, and a magnetic field is measured. The magnetic shield unit is provided with the first opening, and the subject can come in and out through the first opening.

The control unit controlling the table is present at a location separated from the first opening. The control unit makes an electric signal flow so as to control the table. A magnetic field or a residual magnetic field is generated due to the electric signal, and becomes noise when detected by the magnetic detection unit. In the application example, since the control unit is present at the location separated from the first opening, the magnetic field or the residual magnetic field generated from the control unit hardly reaches the magnetic detection unit. As a result, the magnetic detection unit can perform measurement with less noise.

### Application Example 15

In the living body magnetic field measurement apparatus according to the application example, the magnetic shield unit may include a tube through which the inside and the outside of the magnetic shield unit communicate with each other, and the tube extends in a direction orthogonal to the first direction.

According to this application example, the magnetic shield unit includes the tube through which the inside and the outside of the magnetic shield unit communicate with each other, and the tube extends in a direction orthogonal to the first direction. A direction of a magnetic vector passing through the tube is orthogonal to the first direction. Therefore, a magnetic vector passing through the tube hardly influences the magnetic detection unit. As a result, the magnetic detection unit can perform measurement with less noise.

### Application Example 16

In the living body magnetic field measurement apparatus according to the application example, the magnetic shield unit may have a tubular shape extending in a second direction orthogonal to the first direction, and the tube extends in the second direction along the magnetic shield unit.

According to this application example, the tube extends in the second direction, and the second direction is orthogonal to the first direction. Therefore, a magnetic vector passing through the tube hardly influences the magnetic detection unit. As a result, the magnetic detection unit can perform measurement with less noise. Since the tube is provided along the magnetic shield unit, the tube is easily fixed to the magnetic shield unit. Thus, it is possible to easily provide the tube.

### Application Example 17

A living body magnetic field measurement method according to this application example includes measuring shapes of a first surface of a subject and a second surface opposing the first surface; calculating a shape of the second surface when a normal direction of the first surface is set to be the same as a direction of a component in a first direction in which a magnetic detection unit detects a magnetic vector distribution; forming a contact surface of a table which is in contact with the subject in a shape corresponding to the shape of the second surface; mounting the subject on the contact surface of the table; and causing the first surface to come close to the magnetic detection unit so that the distribution of the component in the first direction of the magnetic vector in the subject is detected.

According to this application example, shapes of the first surface and the second surface of the subject are measured. The second surface opposes the first surface. The first surface is a surface for detecting the strength of a magnetic vector in the magnetic detection unit, and the second surface is a surface on which the subject is in contact with the contact surface of the table. The magnetic detection unit detects the component in the first direction of the magnetic vector on the first surface. Next, a shape of the second surface when a normal direction of the first surface is set to be the same as the component in the first direction is calculated. In the calculation, a position and a tilt of the shape of the second surface obtained when the subject is mounted on the table are calculated.

Next, the contact surface of the table is formed in a shape corresponding to the shape with the calculated tilt. Successively, the subject is mounted on the contact surface of the table. The contact surface has the shape corresponding to the shape of the second surface of the subject, and the subject is mounted so that the second surface thereof is in contact with the contact surface. At this time, the first surface of the subject is directed in the first direction. Next, the first surface is caused to come close to the magnetic detection unit. It is possible to increase the sensitivity of the magnetic detection unit through the approach. The distribution of the component in the first direction of the magnetic vector in the subject is detected.

Through the above-described procedures, the first direction can match the normal direction of the first surface of the subject. If the normal direction of the first surface is inclined with respect to the first direction, there may be the occurrence of a location where the distance between the first surface and the magnetic detection unit is short and a location where the distance therebetween is long. The weaker strength of a magnetic vector is detected at the location where the distance between the first surface and the magnetic detection unit is short than a vector at the location where the distance therebetween is long, and thus detection accuracy is reduced. In the application example, the detection strength of a magnetic vector in the first surface can be made uniform. As a result, it is possible to detect the distribution of the magnetic vector of the subject with high accuracy.

### Application Example 18

A living body magnetic field measurement apparatus according to this application example includes a magnetic detection unit that detects a magnetic field coming out of a measured surface of a subject; a position measurement unit that measures a position of the measured surface in a first direction relative to the magnetic detection unit; a table on which the subject is mounted and that moves the subject; and a control unit that controls the table so that a distance in the first direction between the measured surface and the magnetic detection unit becomes a predetermined distance.

According to this application example, the living body magnetic field measurement apparatus includes the magnetic detection unit, the position measurement unit, the table, and the control unit. The magnetic detection unit detects a magnetic vector coming out of a measured surface of a subject. The position measurement unit measures a position of the measured surface in the first direction. The subject is mounted on the table, and the table moves the subject. The control unit controls a position of the table. The control unit controls a distance by which the table is moved on the basis of data regarding a position of the measured surface relative to the magnetic detection unit, measured by the position measurement unit. The control unit performs control so that the distance in the first direction between the measured surface and the magnetic detection unit becomes a predetermined distance. If the magnetic detection unit becomes distant from the measured surface, the strength of a magnetic field detected by the magnetic detection unit is in inverse proportion to the square of a distance from the measured surface. Therefore, detection performance of the magnetic detection unit is reduced as the magnetic detection unit becomes more distant from the measured surface. Since the magnetic detection unit vibrates when the measured surface is in contact with the magnetic detection unit, the measurement accuracy is reduced. In the application example, the measured surface can be made to come close to the magnetic detection unit in a range in which the measured surface is not in contact with the magnetic detection unit. The position measurement unit measures a position of the measured surface relative to the magnetic detection unit, and then the table causes the subject to come close to the magnetic detection unit. Therefore, even if the position measurement unit is separated from the magnetic detection unit, the subject can be made to come close to the magnetic detection unit. As a result, the living body magnetic field measurement apparatus can detect a magnetic field of the measured surface with high accuracy.

### Application Example 19

In the living body magnetic field measurement apparatus according to the application example, the table may move the subject in a second direction and a third direction, the second direction and the third direction may be orthogonal to the first direction, and the second direction and the third direction may intersect each other.

According to this application example, the table moves the subject in the second direction and the third direction. The second direction and the third direction are orthogonal to the first direction. The second direction and the third direction intersect each other. Therefore, the table can move the subject in a direction along a plane orthogonal to the first direction. As a result, the table can easily position the subject in the plane direction orthogonal to the first direction.

### Application Example 20

In the living body magnetic field measurement apparatus according to the application example, the second direction may be orthogonal to the third direction.

According to this application example, the second direction is orthogonal to the third direction. The table moves the subject in the second direction and the third direction orthogonal to each other. Therefore, the table can be moved according to the orthogonal coordinate system, and thus it is possible to easily control a movement position of the table.

### Application Example 21

The living body magnetic field measurement apparatus according to the application example may further include a magnetic shield unit that encloses the magnetic detection unit, includes a first opening through which the table comes in and out in the second direction, and attenuates an entering magnetic field line.

According to this application example, the living body magnetic field measurement apparatus includes the magnetic shield unit. The magnetic shield unit attenuates an entering magnetic field line. The magnetic detection unit is provided inside the magnetic shield unit so as to measure a magnetic field. The magnetic shield unit includes a first opening and attenuates an entering magnetic field line. Consequently, the magnetic detection unit can perform measurement with less noise.

### Application Example 22

In the living body magnetic field measurement apparatus according to the application example, the position measurement unit may measure a single location on the measured surface whose height from the table is large.

According to this application example, the position measurement unit measures a single location on the measured surface of which a height from the table is large. Therefore, it is possible to detect a position of a most protruding portion of the measured surface. As a result, the measured surface can be made to come close to the magnetic detection unit in a range in which the most protruding portion does not come into contact with the magnetic detection unit.

### Application Example 23

In the living body magnetic field measurement apparatus according to the application example, the position measurement unit may measure a stereoscopic shape of the measured surface.

According to this application example, the position measurement unit measures a stereoscopic shape of the measured surface. Therefore, it is possible to detect a position of a most protruding portion of the measured surface. As a result, the measured surface can be made to come close to the magnetic detection unit in a range in which the most protruding portion does not come into contact with the magnetic detection unit.

### Application Example 24

In the living body magnetic field measurement apparatus according to the application example, the position measurement unit may scan the measured surface with a light beam, and measures a location irradiated with the light beam.

According to this application example, the position measurement unit scans the measured surface with a light beam. A location irradiated with the light beam is measured. Therefore, the position measurement unit can detect a position of a most protruding portion within a range in which scanning is performed with the light beam.

### Application Example 25

The living body magnetic field measurement apparatus according to the application example may further include a guide light irradiation unit that applies a light beam for guiding a position where the subject is mounted, the position measurement unit irradiates the subject with a light beam and performs measurement, the position measurement unit is also used as the guide light irradiation unit, and the position measurement unit applies a light beam for guiding a position where the subject is mounted.

According to this application example, the living body magnetic field measurement apparatus has a function of a guide light irradiation unit and a function of a position measurement unit. The function of the guide light irradiation unit is a function of applying a light beam for guiding a position where the subject is mounted. The function of the position measurement unit is a function of irradiating the subject with a light beam so as to measure a shape of the subject. The position measurement unit is also used as the guide light irradiation unit, and the position measurement unit applies a light beam for guiding a position where the subject is mounted. Therefore, it is possible to reduce the number of constituent elements compared with a case where the living body magnetic field measurement apparatus separately includes the guide light irradiation unit and the position measurement unit. As a result, it is possible to manufacture the living body magnetic field measurement apparatus with high productivity.

### Application Example 26

In the living body magnetic field measurement apparatus according to the application example, the position measurement unit may be provided in the first opening.

According to this application example, the position measurement unit is provided in the first opening. The subject mounted on the table passes through the first opening. Therefore, the subject passes near the position measurement unit, and thus the position measurement unit can easily irradiate the subject with light.

### Application Example 27

In the living body magnetic field measurement apparatus according to the application example, a portion of the table which may be moved into the magnetic shield unit is non-magnetic.

According to this application example, a portion of the table which can be moved into the magnetic shield unit is non-magnetic. Therefore, it is possible to prevent magnetization of the table from influencing measurement of a magnetic field.

### Application Example 28

In the living body magnetic field measurement apparatus according to the application example, the control unit may be present at a location separated from the first opening.

According to this application example, the living body magnetic field measurement apparatus includes the magnetic shield unit. The magnetic shield unit attenuates an entering magnetic field line. The magnetic detection unit and the table are provided inside the magnetic shield unit, and a magnetic field is measured. The magnetic shield unit is provided with the first opening, and the subject can come in and out through the first opening.

The control unit controlling the table is present at a location separated from the first opening. The control unit makes an electric signal flow so as to control the table. A magnetic field or a residual magnetic field is generated due to the electric signal, and becomes noise when detected by the magnetic detection unit. In the application example, since the control unit is present at the location separated from the first opening, the magnetic field or the residual magnetic field generated from the control unit hardly reaches the magnetic detection unit. As a result, the magnetic detection unit can perform measurement with less noise.

### Application Example 29

In the living body magnetic field measurement apparatus according to the application example, the magnetic shield unit may include a tube through which the inside and the outside of the magnetic shield unit communicate with each other, and the tube may extend in a direction orthogonal to the first direction.

According to this application example, the magnetic shield unit includes the tube through which the inside and the outside of the magnetic shield unit communicate with each other, and the tube extends in a direction orthogonal to the first direction. A direction of a magnetic vector passing through the tube is orthogonal to the first direction. Therefore, a magnetic vector passing through the tube hardly influences the magnetic detection unit. As a result, the magnetic detection unit can perform measurement with less noise.

### Application Example 30

The living body magnetic field measurement apparatus according to the application example may further include a driving source that moves the table in the third direction, and the driving source may include an attachment/detachment portion that is located outside the magnetic shield unit and attaches or detaches the table and the driving source to each other or from each other.

According to this application example, the driving source moves the table in the third direction. The driving source includes the attachment/detachment portion that is located outside the magnetic shield unit and attaches or detaches the table and the driving source to each other or from each other. Therefore, the attachment/detachment portion connects the table to the driving source, and thus the table can be moved in the third direction by using the driving source. When the table is not moved in the third direction, the attachment/detachment portion can detach the driving source from the table. The driving source is located outside the magnetic shield unit, and can move the table into the magnetic shield unit. Therefore, it is possible for the inside of the magnetic shield unit to be hardly influenced by a magnetic field of the driving source. As a result, the magnetic detection unit can perform measurement with less noise.

### Application Example 31

The living body magnetic field measurement apparatus according to the application example may further include a driving source that moves the table in the second direction, and the driving source is located outside the magnetic shield unit.

According to this application example, the driving source moves the table in the second direction. The driving source is located outside the magnetic shield unit. Therefore, it is possible for the inside of the magnetic shield unit to be hardly influenced by a magnetic field of the driving source. As a result, the magnetic detection unit can perform measurement with less noise.

### Application Example 32

A living body magnetic field measurement method according to this application example includes mounting a subject on a table; causing a position measurement unit to measure a stereoscopic shape of a measured surface of the subject; calculating a most protruding portion of the stereoscopic shape; moving the table so that the most protruding portion comes close to a magnetic detection unit with a predetermined gap; and causing the magnetic detection unit to detect a distribution of a magnetic vector in the subject.

According to this application example, the subject is mounted on the table, and a stereoscopic shape of a measured surface of the subject is measured. A most protruding portion of the stereoscopic shape is calculated. Next, the table is moved so that the most protruding portion comes close to the magnetic detection unit with a predetermined gap. Successively, a distribution of a magnetic vector in the subject is detected. Therefore, the magnetic detection unit approaches and measures the measured surface in a range in which the magnetic detection unit does not come into contact with the measured surface. The position measurement unit measures a position of the measured surface relative to the magnetic detection unit, and then the table causes the subject to come close to the magnetic detection unit. Thus, even if the position measurement unit is separated from the magnetic detection unit, the subject can be made to come close to the magnetic detection unit. As a result, the living body magnetic field measurement apparatus can measure a magnetic field of the measured surface with high accuracy.

### Application Example 33

In the living body magnetic field measurement method according to the application example, in a case where the stereoscopic shape is measured, a position of the subject in a direction orthogonal to a longitudinal direction of the subject is set, and, in a case where the table is moved, the table may be moved in the longitudinal direction of the subject.

According to this application example, in a case where the stereoscopic shape is measured, first, a position of the subject in a direction orthogonal to a longitudinal direction of the subject is set. Consequently, it is possible to reliably measure a measurement region. Next, the table is moved in the longitudinal direction of the subject. Consequently, it is possible to measure a two-dimensional measurement region.

### Application Example 34

In the living body magnetic field measurement apparatus according to the application example, the magnetic shield unit may have a tubular shape extending in the second direction, and the tube may extend in the second direction along the magnetic shield unit.

According to this application example, the tube extends in the second direction, and the second direction is orthogonal to the first direction. Therefore, a magnetic vector passing through the tube hardly influences the magnetic detection unit. As a result, the magnetic detection unit can perform measurement with less noise. Since the tube is provided along the magnetic shield unit, the tube is easily fixed to the magnetic shield unit. Thus, it is possible to easily provide the tube.

### BRIEF DESCRIPTION OF THE DRAWINGS

The invention will be described with reference to the accompanying drawings, wherein like numbers reference like elements.

FIG. 1 is a schematic perspective view illustrating a configuration of a magnetic field measurement apparatus according to a first embodiment.

FIG. 2A is a schematic side sectional view for explaining a structure of a shape measurement device, and FIG. 2B is a schematic side view for explaining a structure of the shape measurement device.

FIG. 3A is a main portion schematic plan view for explaining an arrangement of a tilting device, and FIG. 3B is a schematic side sectional view for explaining a structure of the shape measurement device.

FIG. 4 is a main portion schematic perspective view illustrating a positional relationship between a measured surface and a magnetic sensor.

FIG. 5A is a schematic side view illustrating a structure of an attachment/detachment portion; FIG. 5B is a side view of a grooved rod; FIG. 5C is a side view of a grooved cylinder; and FIG. 5D is a schematic side view illustrating a structure of the attachment/detachment portion.

FIG. 6A is a schematic side view illustrating a structure of the magnetic sensor, and FIG. 6B is a schematic plan view illustrating a structure of the magnetic sensor.

FIG. 7 is an electrical control block diagram of a controller.

FIG. 8 is a flowchart illustrating a magnetic field measurement method.

FIGS. 9A to 9C are schematic diagrams for explaining the magnetic field measurement method.

FIGS. 10A to 10C are schematic diagrams for explaining the magnetic field measurement method.

FIGS. 11A and 11B are schematic diagrams for explaining the magnetic field measurement method.

FIGS. 12A to 12C are schematic diagrams for explaining the magnetic field measurement method.

FIG. 13 is a schematic perspective view illustrating a configuration of a living body magnetic field measurement apparatus according to a second embodiment.

FIG. 14A is a schematic side view illustrating a structure of a contour measurement section, and FIG. 14B is a schematic top view illustrating a structure of the contour measurement section.

FIGS. 15A and 15B are schematic sectional views illustrating a structure of the table.

FIG. 16A is a side view illustrating a structure of an X-direction table; FIG. 16B is a main portion schematic enlarged view for explaining a movable range of the division surface; FIG. 16C is a top sectional view for explaining a configuration of a tube; and FIG. 16D is a side sectional view for explaining a configuration of the tube.

FIG. 17A is a schematic side view illustrating a structure of a magnetic sensor, and FIG. 17B is a schematic plan view illustrating a structure of the magnetic sensor.

FIG. 18 is an electrical control block diagram of a controller.

FIG. 19 is a flowchart illustrating a living body magnetic field measurement method.

FIGS. 20A to 20E are schematic diagrams for explaining the living body magnetic field measurement method.

FIGS. 21A to 21C are schematic diagrams for explaining the living body magnetic field measurement method.

FIGS. 22A and 22B are schematic diagrams for explaining the living body magnetic field measurement method.

FIG. 23 is a schematic perspective view illustrating a configuration of a living body magnetic field measurement apparatus according to a fourth embodiment.

FIGS. 24A and 24B are schematic sectional views for explaining a structure of the position measurement device.

FIG. 25A is a perspective view of a three-dimensional image measured by the position measurement device, and FIG. 25B is a schematic side view of a stereoscopic image for explaining measurement in the position measurement device.

FIGS. 26A and 26B are schematic side sectional views illustrating a structure of the table.

FIG. 27A is a schematic side view illustrating a structure of an attachment/detachment portion; FIG. 27B is a side view of a grooved rod; FIG. 27C is a side view of a grooved cylinder; and FIG. 27D is a schematic side view illustrating a structure of the attachment/detachment portion.

FIG. 28A is a top sectional view for explaining a configuration of a tube, and FIG. 28B is a side sectional view for explaining a configuration of the tube.

FIG. 29A is a schematic side view illustrating a structure of a magnetic sensor, and FIG. 29B is a schematic plan view illustrating a structure of the magnetic sensor.

FIG. 30 is an electrical control block diagram of a controller.

FIG. 31 is a flowchart illustrating a living body magnetic field measurement method.

FIGS. 32A to 32C are schematic diagrams for explaining the living body magnetic field measurement method.

FIGS. 33A to 33C are schematic diagrams for explaining the living body magnetic field measurement method.

### DESCRIPTION OF EXEMPLARY EMBODIMENTS

In the present embodiments, with reference to the drawings, a description will be made of characteristic examples of a magnetic field measurement apparatus and a magnetic field measurement method of measuring a heart magnetic field generated from the heart by using the magnetic field measurement apparatus. In addition, respective members in the drawings are illustrated in different scales in order to be recognizable in the drawings.

### First Embodiment

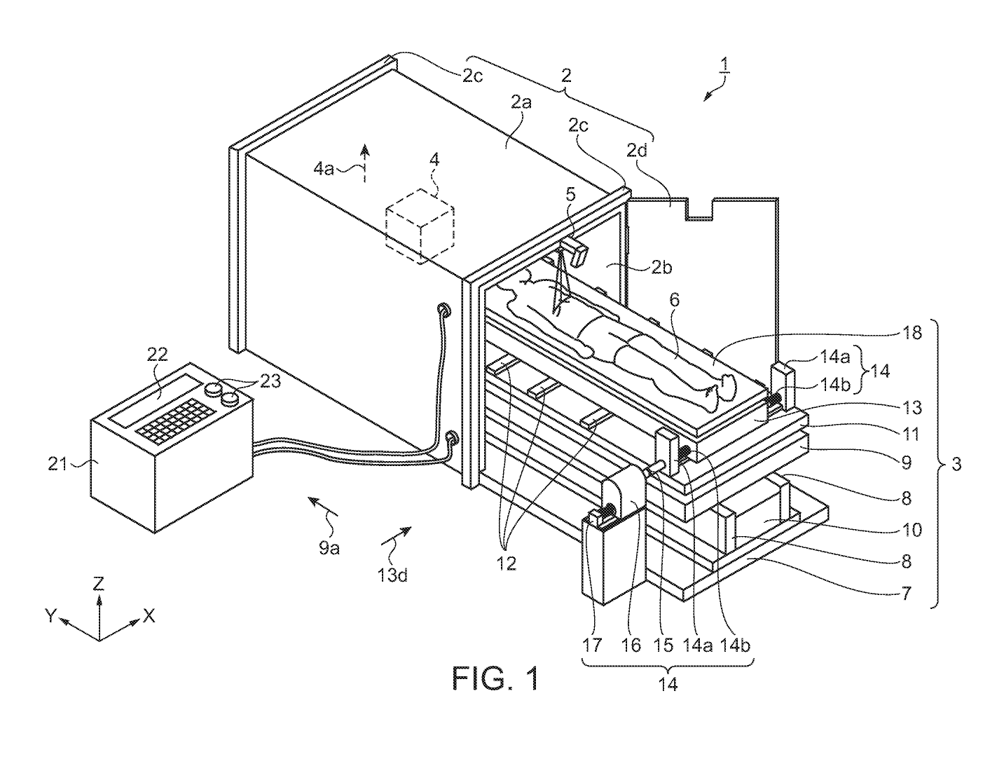

With reference to FIGS. 1 to 7, a description will be made of a structure of a magnetic field measurement apparatus according to a first embodiment. FIG. 1 is a schematic perspective view illustrating a configuration of the magnetic field measurement apparatus. As illustrated in FIG. 1, a magnetic field measurement apparatus 1 mainly includes an electromagnetic shield device 2 as a magnetic shield unit, a table 3 as a movable table, a magnetic sensor 4 as a detection unit, and a shape measurement device 5 as a position measurement unit, a guide light irradiation unit, and a measurement unit.

The electromagnetic shield device 2 includes a rectangular tubular main body 2a. A longitudinal direction of the main body 2a is set to a Y direction. The gravity direction is set to a −Z direction, and a direction orthogonal to the Y direction and the Z direction is set to an X direction. The electromagnetic shield device 2 prevents an external magnetic field such as terrestrial magnetism from entering a space where the magnetic sensor 4 is disposed. In other words, the electromagnetic shield device 2 attenuates a magnetic force line entering the inside. That is, the influence of the external magnetic field on the magnetic sensor 4 is minimized by the electromagnetic shield device 2, and a magnetic field in the location where the magnetic sensor 4 is present is considerably lower than the external magnetic field. The main body 2a extends in the Y direction, and the main body 2a functions as a passive magnetic shield. The inside of the main body 2a is hollow, and a sectional shape of surfaces passing through the X direction and the Z direction is a substantially quadrangle shape. The surfaces passing through the X direction and the Z direction indicate orthogonal planes orthogonal in the Y direction in the XZ section.

A sectional shape of the main body 2a is a square shape. The electromagnetic shield device 2 is provided with an opening 2b on the −Y direction side, and the table 3 protrudes out of the opening 2b. The size of the electromagnetic shield device 2 is not particularly limited, but, in the present embodiment, for example, a length thereof in the Y direction is about 200 cm, and one side of the opening 2b is about 90 cm. A subject 6 mounted on the table 3 can come in and out of the electromagnetic shield device 2 via the opening 2b along with the table 3. In the present embodiment, the subject 6 is a human, but an animal other than a human may be used as the subject 6. A door 2d is provided in the opening 2b via so as to be opened and closed via hinges. The opening 2b may be closed by the door 2d after the subject 6 and the table 3 enter the main body 2a. The door 2d can reduce a magnetic field entering the main body 2a.

The main body 2a and the door 2d are made of a ferromagnetic material having relative permeability of, for example, several thousands or more, or a conductor having high conductivity. As the ferromagnetic material, permalloy, ferrite, iron, chromium, cobalt-based amorphous metal, or the like may be used. As the conductor having high conductivity, for example, aluminum which has a magnetic field reduction function due to an eddy current effect may be used. The main body 2a may be formed by alternately stacking a ferromagnetic material and a conductor having high conductivity. In the present embodiment, for example, each of the main body 2a and the door 2d is formed by alternately staking an aluminum plate and a permalloy plate as two layers whose entire thickness is about 20 mm to 30 mm.

First Helmholtz coils 2c are provided at ends on the +Y direction side and the −Y direction side of the main body 2a. The first Helmholtz coils 2c are coils for correcting an entering magnetic field which enters the internal space of the main body 2a. The entering magnetic field indicates an external magnetic field which passes through the opening 2b and enters the internal space. The entering magnetic field is strongest with respect to the opening 2b in the Y direction. The first Helmholtz coils 2c generate a magnetic field which cancels out the entering magnetic field by using a current.

The table 3 is provided with a foundation 7. The foundation 7 is disposed on the bottom inside the main body 2a, and extends from the inside of the main body 2a to the outside of the opening 2b through the opening 2b in the Y direction. The foundation 7 extends in a direction in which the subject 6 is movable. A pair of Y-direction rails 8 extending in the Y direction is provided on the foundation 7. A Y-direction table 9 which is moved in the Y direction as a second direction 9a along the Y-direction rails 8 is provided on the Y-direction rails 8. A Y-direction linear motion mechanism 10 which moves the Y-direction table 9 is provided between the two Y-direction rails 8.

A Z-direction table 11 is provided on the Y-direction table 9, and a lifting device (not illustrated) is provided between the Y-direction table 9 and the Z-direction table 11. The lifting device lifts the Z-direction table 11. Six X-direction rails 12 extending in the X direction are provided on a surface on the +Z direction side of the Z-direction table 11. An X-direction table 13 which is moved in the X direction along the X-direction rails 12 is provided on the X-direction rails 12.

An X-direction linear movement mechanism 14 which moves the X-direction table 13 in the X direction as a third direction 13d is provided on the −Y direction side on the Z-direction table 11. The X-direction linear movement mechanism 14 includes a pair of bearing portions 14a, and the bearing portions 14a are provided to be erect on the Z-direction table 11. The X-direction table 13 is located between the two bearing portions 14a. The two bearing portions 14a rotatably support a first screw rod 14b. A first penetration hole (not illustrated) which penetrates in the X direction is provided in the X-direction table 13, and the first screw rod 14b is provided to penetrate through the first penetration hole of the X-direction table 13. A female screw (not illustrated) is formed on the first penetration hole, and the first screw rod 14b is engaged with the female screw.

An attachment/detachment portion 15 is provided at one end on the −X direction side of the first screw rod 14b, and the attachment/detachment portion 15 is fixed to the first screw rod 14b. If the attachment/detachment portion 15 is rotated, the first screw rod 14b is rotated. Since the first screw rod 14b is engaged with the female screw of the X-direction table 13, if the first screw rod 14b is rotated, the X-direction table 13 is moved in the X direction. The attachment/detachment portion 15 is coupled to a rotation shaft of an X-direction table motor 16. The X-direction table motor 16 rotates the attachment/detachment portion 15 so as to move the X-direction table 13 in the X direction. The X-direction table motor 16 is coupled to a motor movement portion 17 which moves the X-direction table motor 16 in the X direction. The X-direction linear movement mechanism 14 is constituted of the bearing portions 14a, the first screw rod 14b, the attachment/detachment portion 15, the X-direction table motor 16, the motor movement portion 17, and the like.

A tilting table 18 is provided over the X-direction table 13, and a tilting device (not illustrated) is provided between the X-direction table 13 and the tilting table 18. The tilting device tilts the tilting table 18 with respect to the X-direction table 13. The foundation 7, the Y-direction rails 8, the Y-direction table 9, the Y-direction linear motion mechanism 10, the Z-direction table 11, the X-direction rails 12, the X-direction table 13, the bearing portions 14a, the first screw rod 14b, and the tilting table 18 constituting the table 3 are made of non-magnetic materials such as a wood, a resin, a ceramic, and non-magnetic metal. A portion of the table 3 which is moved to the inside of the electromagnetic shield device 2 is made of a non-magnetic material. Consequently, it is possible to prevent magnetization of the table 3 from influencing measurement of a magnetic field.

In the electromagnetic shield device 2, the shape measurement device 5 is provided on the +Z direction side of the opening 2b. The shape measurement device 5 is a device used to position the subject 6 or measure a surface shape. The subject 6 mounted on the table 3 passes through the opening 2b. The subject 6 passes near the shape measurement device 5, and thus the shape measurement device 5 can easily irradiate the subject 6 with light beams. The shape measurement device 5 detects light reflected from the subject 6 so as to measure a shape of the subject 6.

The magnetic sensor 4 is provided inside the electromagnetic shield device 2. The magnetic sensor 4 is a sensor which detects a magnetic field generated from the heart of the subject 6. The magnetic sensor 4 is fixed to the electromagnetic shield device 2. The location where the magnetic field measurement apparatus 1 is disposed is adjusted to a state in which no magnetic field is substantially present by the electromagnetic shield device 2. Therefore, the magnetic sensor 4 can measure a magnetic field generated from the heart without being influenced by noise. The magnetic sensor 4 detects an intensity component of a magnetic field in a first direction 4a which is the same direction as the Z direction.

The first direction 4a and the second direction 9a are directions orthogonal to each other. The first direction 4a and the third direction 13d are directions orthogonal to each other. The second direction 9a and the third direction 13d are also directions orthogonal to each other. The table 3 moves the subject 6 in the second direction 9a and the third direction 13d orthogonal to each other. The table 3 is moved in an orthogonal coordinate system, and can thus easily control a position of the subject 6. The table 3 controls a tilt angle of the subject 6. A direction in which the electromagnetic shield device 2 extends is the second direction 9a.

A controller 21 is provided at a location separated from the opening 2b. The controller 21 outputs an electric signal so as to control the magnetic field measurement apparatus 1. Specifically, the controller 21 controls the electromagnetic shield device 2, the table 3, the magnetic sensor 4, and the shape measurement device 5. A magnetic field or a residual magnetic field is generated due to the electric signal of the controller 21, and becomes noise when detected by the magnetic sensor 4. Since the controller 21 is present at the location separated from the opening 2b, the magnetic field or the residual magnetic field generated from the controller 21 hardly reaches the magnetic sensor 4. As a result, the magnetic sensor 4 can perform measurement with less noise.

The controller 21 is provided with a display device 22 and an input device 23. The display device 22 is a liquid crystal display (LCD) or an organic light emitting diode (OLED). A measurement situation, a measurement result, and the like are displayed on the display device 22. The input device 23 is constituted of a keyboard, a rotary knob, or the like. An operator operates the input device 23 so as to input various instructions such as a measurement starting instruction or a measurement condition to the magnetic field measurement apparatus 1.

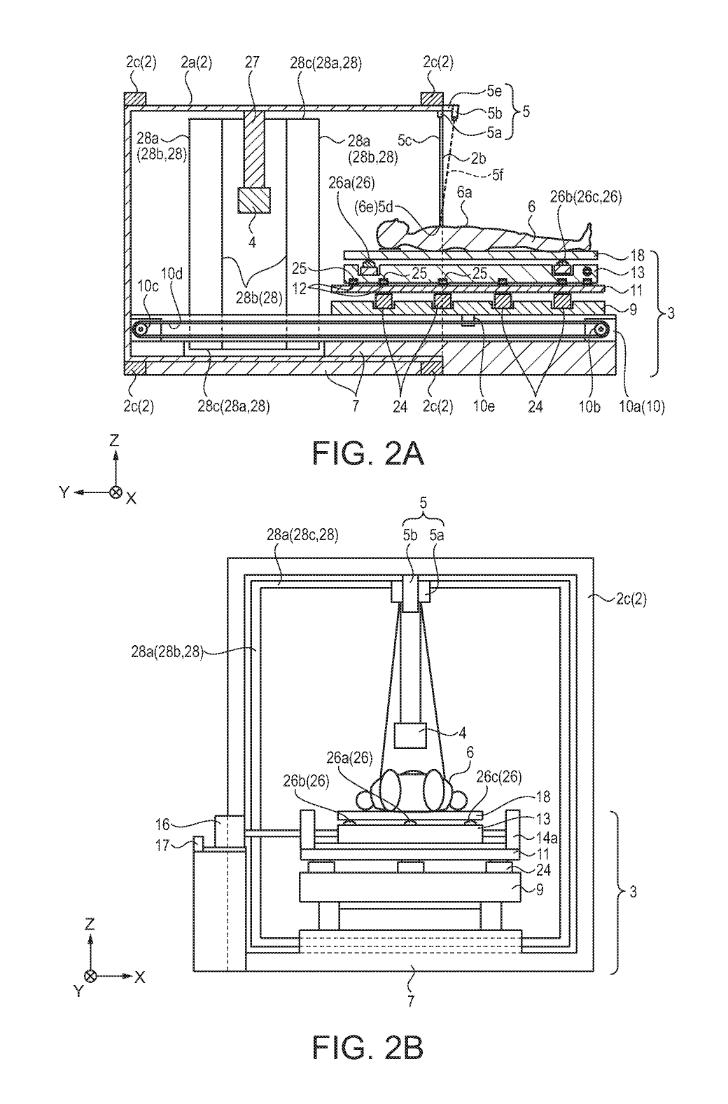

FIG. 2A is a schematic sectional view for explaining a structure of the shape measurement device, and is a view taken along the side surface of the electromagnetic shield device 2. FIG. 2B is a schematic sectional view for explaining a structure of the shape measurement device, and is a view in which the magnetic field measurement apparatus 1 is viewed from the −Y direction side. In FIGS. 2A and 2B, the shape measurement device 5 includes a laser scanning unit 5a and an imaging device 5b as a guide light irradiation unit. The laser scanning unit 5a is provided on a ceiling of the main body 2a in the opening 2b, and emits laser light 5c as light and a light beam in the −Z direction. A front face 6a of the subject 6 is irradiated with the laser light 5c. The laser light 5c is reflected from the front face 6a. The laser scanning unit 5a has a function of performing scanning with the laser light 5c in the X direction, and a function of irradiating a single point without scanning. When the laser scanning unit 5a performs scanning with the laser light 5c, a reflection point 5d at which the laser light 5c is reflected from the front face 6a is linear when viewed from the imaging device 5b. When the laser scanning unit 5a does not perform scanning with the laser light 5c, the reflection point 5d at which the laser light 5c is reflected from the front face 6a is a single point.

When the subject 6 is positioned, the subject 6 is mounted on the table 3 so as to be directed upward. The laser scanning unit 5a irradiates the chest of the subject 6 without scanning with the laser light 5c. The operator drives the Y-direction linear motion mechanism 10 so as to move the Y-direction table 9 in the Y direction. The operator drives the X-direction linear movement mechanism 14 and the X-direction table motor 16 so as to move the X-direction table 13 in the X direction. Positions of the table 3 in the X direction and the Y direction are adjusted so that the laser light 5c is applied to the xiphisternum 6e of the subject 6.

The shape measurement device 5 has a function of applying the laser light 5c as guide light, and a function of measuring a position. The function of applying guide light is a function of applying a light beam for guiding a position where the subject 6 is mounted. The function of measuring a position is a function of measuring a shape of the subject by irradiating the subject 6 with a light beam. The shape measurement device 5 has the function of applying guide light, and thus the shape measurement device 5 applies a light beam for guiding a position where the subject 6 is mounted. Therefore, it is possible to reduce the number of constituent elements compared with a case where the magnetic field measurement apparatus 1 separately includes a constituent element applying guide light and a constituent element measuring a position.

The imaging device 5b is provided in the opening 2b of the main body 2a via a support portion 5e. The imaging device 5b is obliquely provided with respect to an advancing direction of the laser light 5c. The imaging device 5b images reflected light 5f which is reflected from the front face 6a of the subject 6. In this case, the laser scanning unit 5a, the reflection point 5d, and the imaging device 5b form a triangle. The distance between the laser scanning unit 5a and the imaging device 5b is a known value. An angle formed between the laser light 5c and the reflected light 5f can be detected on the basis of an image captured by the imaging device 5b. Therefore, the shape measurement device 5 can measure the distance between the laser scanning unit 5a and the reflection point 5d by using a triangulation method. As mentioned above, the shape measurement device 5 irradiates the subject 6 with the laser light 5c so as to measure a location irradiated with the laser light 5c. A surface of the subject 6 is uneven, and the laser light 5c is reflected at the uneven surface. Therefore, the shape measurement device 5 can easily detect a surface shape of the subject 6 by detecting a position of the laser light 5c reflected from the subject 6.

The pair of first Helmholtz coils 2c is provided at the foundation 7. A shape of each of the first Helmholtz coils 2c is a quadrangle frame shape, and the first Helmholtz coils 2c are disposed to surround the main body 2a. A part of the first Helmholtz coil 2c on the −Z direction side is located in the inner part of the foundation 7. The main body 2a is installed inside the first Helmholtz coils 2c. Consequently, the first Helmholtz coils 2c have a structure of surrounding the main body 2a over the entire circumference thereof.

The Y-direction linear motion mechanism 10 includes a motor 10a as a driving source. A first pulley 10b is provided on a rotation shaft of the motor 10a, and a second pulley 10c is rotatably provided at an end on the Y direction side of the Y-direction linear motion mechanism 10. A timing belt 10d is hung on the first pulley 10b and the second pulley 10c. A connection portion 10e is provided on the timing belt 10d, and the connection portion 10e connects the timing belt 10d to the Y-direction table 9. When the motor 10a rotates the first pulley 10b, the connection portion 10e is moved in the Y direction by the torque of the motor 10a. The Y-direction table 9 is moved due to the movement of the connection portion 10e. Therefore, the motor 10a can move the Y-direction table 9 in the Y direction. The motor 10a changes a rotation direction of the first pulley 10b so as to move the Y-direction table 9 in both directions such as +Y direction and the −Y direction.

Materials of the Y-direction rails 8, the second pulley 10c, the timing belt 10d, and the connection portion 10e are non-magnetic materials. The timing belt 10d is made of rubber and resin. The Y-direction rails 8, the second pulley 10c, and the connection portion 10e are made of ceramics. Therefore, a portion of the Y-direction linear motion mechanism 10 entering the electromagnetic shield device 2 is non-magnetic.

Four lifting devices 24 are provided side by side in the Y direction in the Y-direction table 9. Each of the lifting devices 24 has a structure in which three air cylinders are arranged in the X direction. The lifting device 24 expands and contracts the air cylinders so as to lift the Z-direction table 11 in the first direction 4a. Each air cylinder is provided with a length measurement device (not illustrated), and thus the lifting device 24 detects a movement amount of the Z-direction table 11. The respective air cylinders move the Z-direction table 11 by the same distance, and thus the lifting devices 24 can move the Z-direction table 11 in parallel. Pneumatic equipment such as a compressor and an electromagnetic valve (not illustrated) is provided in the controller 21. The lifting devices 24 are controlled by the controller 21. The Y-direction table 9, the lifting devices 24, and the Z-direction table 11 are made of aluminum. Therefore, the Y-direction table 9, the lifting devices 24, and the Z-direction table 11 are non-magnetic.

The X-direction table 13 is provided with wheels 25 which are in contact with the X-direction rails 12. The wheels 25 are rotated, and thus the X-direction table 13 can be easily moved in the X direction. The X-direction table 13, the X-direction rails 12, and the wheels 25 are made of ceramics which are non-magnetic materials. Therefore, the X-direction table 13, the X-direction rails 12, and the wheels 25 are non-magnetic. A portion of the table 3 entering the electromagnetic shield device 2 is non-magnetic. Consequently, it is possible to prevent magnetization of the table 3 from influencing measurement of a magnetic field.

A tilting device 26 as a leg portion is provided between the X-direction table 13 and the tilting table 18. The tilting device 26 includes a first tilting portion 26a, a second tilting portion 26b, and a third tilting portion 26c. The first tilting portion 26a to the third tilting portion 26c have the same structure as that of the lifting device 24. The controller 21 controls a length of expansion/contraction of each of the first tilting portion 26a to the third tilting portion 26c. An X-direction table 13 side of the first tilting portion 26a is fixed to the X-direction table 13. The first tilting portion 26a on a tilting table 18 side has a conical shape, and is coupled to the tilting table 18 via a pivot bearing.

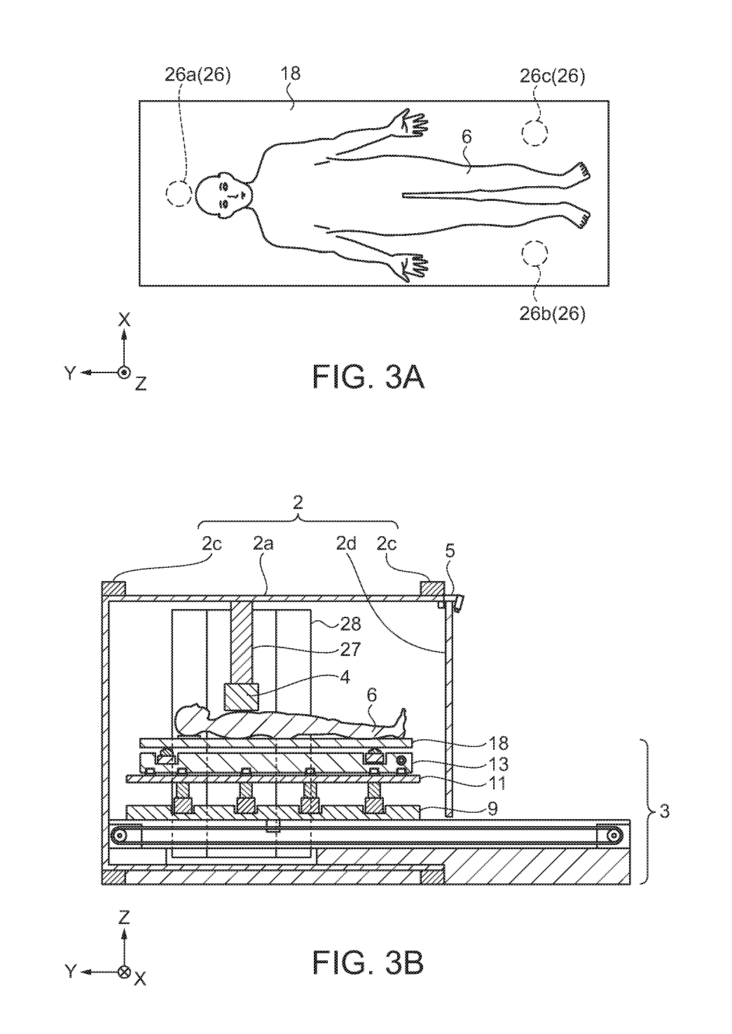

Both of an X-direction table 13 side and a tilting table 18 side of each of the second tilting portion 26b and the third tilting portion 26c have a conical shape. The second tilting portion 26b and the third tilting portion 26c are coupled to the X-direction table 13 and the tilting table 18 via pivot bearings. FIG. 3A is a main portion schematic plan view for explaining arrangement of the tilting device. As illustrated in FIG. 3A, the first tilting portion 26a is provided on the +Y direction side of the tilting table 18. The second tilting portion 26b is provided on the −X direction side of the −Y direction side of the tilting table 18. The third tilting portion 26c is provided on the +X direction side of the −Y direction side of the tilting table 18. A line connecting the first tilting portion 26a to the third tilting portion 26c to each other forms an isosceles triangle.

When the first tilting portion 26a is expanded or contracted, the tilting table 18 is rotated with a line connecting the second tilting portion 26b to the third tilting portion 26c as an axis. When the second tilting portion 26b is expanded or contracted, the tilting table 18 is rotated with a line connecting the first tilting portion 26a to the third tilting portion 26c as an axis. When the third tilting portion 26c is expanded or contracted, the tilting table 18 is rotated with a line connecting the first tilting portion 26a to the second tilting portion 26b as an axis. The controller 21 controls an expansion/contraction amount of each of the first tilting portion 26a to the third tilting portion 26c so as to control a tilt angle of the tilting table 18. In other words, the controller 21 controls lengths of the first tilting portion 26a to the third tilting portion 26c so as to tilt the subject 6.

As one of structures of a device tilting the tilting table 18, there is a structure in which a tilting device controlling a tilt is provided at the center of the tilting table 18. On the other hand, in the table 3 of the present embodiment, the first tilting portion 26a to the third tilting portion 26c have a structure of supporting the tilting table 18. When compared with the structure in which the tilting device is provided at the center, loads of the tilting table 18 and the subject 6 can be distributed to the first tilting portion 26a to the third tilting portion 26c in the table 3, and thus it is possible to control a tilt of the tilting table 18 with a lightweight structure.

Referring to FIGS. 2A and 2B again, the magnetic sensor 4 is provided on the ceiling of the main body 2a via a support member 27. A position of the center of the magnetic sensor 4 in the Z direction is the central position between the ceiling of the main body 2a and the bottom of the main body 2a. A position of the center of the magnetic sensor 4 in the X direction is the central position between a wall on the +X direction side of the main body 2a and a wall on the −X direction side thereof. The distance between the center of the magnetic sensor 4 in the Y direction and an end on the −Y direction side of the main body 2a is the same as the distance between the center of the magnetic sensor 4 and the wall on the +Y direction side of the main body 2a. When the center of the magnetic sensor 4 is located at this location, it is possible for the magnetic sensor 4 to be hardly influenced by a magnetic field which enters the electromagnetic shield device 2 from the outside thereof.

A second Helmholtz coil 28 having a cube frame shape is provided inside the electromagnetic shield device 2. Specifically, at least three correction coils are provided in the second Helmholtz coil 28 so as to be orthogonal to each other in the X direction, the Y direction, and the Z direction. In an X-direction correction coil 28a orthogonal to the X direction, a measurement space in which the subject 6 is disposed during measurement and the magnetic sensor 4 are interposed between a pair of coils from the X direction. The X-direction correction coil 28a generates a magnetic field in the X direction and cancels out an external magnetic field in the X direction so that an X component of a magnetic field of the measurement space and the space in which the magnetic sensor 4 is disposed is reduced to the extent or less of not having an adverse effect on measurement.

Two pairs of coils are provided in a Y-direction correction coil 28b orthogonal to the Y direction, and the measurement space and the magnetic sensor 4 are interposed between the coils of the Y-direction correction coil 28b from the Y direction. The Y-direction correction coil 28b includes two pairs of coils, and thus includes four coils. The Y-direction correction coil 28b generates a magnetic field in the Y direction and cancels out an external magnetic field in the Y direction so that a Y component of a magnetic field of the measurement space and the space in which the magnetic sensor 4 is disposed is reduced to the extent or less of not having an adverse effect on measurement. The main body 2a has a tubular shape extending in the Y direction, and an entering magnetic field is considerable in the Y direction. For this reason, the Y-direction correction coil 28b includes the two pairs of coils.

In a Z-direction correction coil 28c orthogonal to the Z direction, a measurement space in which the subject 6 is disposed during measurement and the magnetic sensor 4 are interposed between a pair of coils from the Z direction. The Z-direction correction coil 28c generates a magnetic field in the Z direction and cancels out an external magnetic field in the Z direction so that a Z component of a magnetic field of the measurement space and the space in which the magnetic sensor 4 is disposed is reduced to the extent or less of not having an adverse effect on measurement.

The second Helmholtz coil 28 has a square frame shape when viewed from each orthogonal direction side, and is disposed so that the central position of the square frame overlaps the central position of the magnetic sensor 4. A length of the side of the square is not particularly limited, but, in the present embodiment, a length of one side is equal to or more than 75 cm and equal to or less than 85 cm. In FIGS. 2A and 2B, the shape of the second Helmholtz coil 28 is a rectangular shape for better viewing, but is actually a square shape.

In the Y-direction correction coil 28b, the four coils are disposed at the same interval in the Y direction. When viewed from the X direction, an outer circumference of the second Helmholtz coil 28 has a square frame shape, and there is a structure in which two coils are disposed inside the square frame shape. The second Helmholtz coil 28 is disposed so that the central position of the square frame shape overlaps the central position of the magnetic sensor 4.

A shape of the second Helmholtz coil 28 viewed from the Z direction is a square frame shape which is the same as the shape viewed from the X direction. The second Helmholtz coil 28 is disposed so that the central position of the square frame shape overlaps the central position of the magnetic sensor 4. The second Helmholtz coil 28 has such a shape, and thus it is possible to further reduce a disturbance magnetic field in the magnetic sensor 4. Particularly, it is possible to reduce the influence of a magnetic flux which comes from the −Y direction side of the electromagnetic shield device 2.

When the table 3 is located on the −Y direction side of the electromagnetic shield device 2, a half or more of the table 3 protrudes out of the electromagnetic shield device 2. Consequently, the subject 6 is easily mounted on the table 3. When the subject 6 is mounted on the table 3, a height from the floor to the nose of the subject 6 is smaller than a height from the floor to the surface on the −Z direction side of the magnetic sensor 4. Therefore, when the Y-direction table 9 is moved in the Y direction, the subject 6 does not interfere with the magnetic sensor 4.

FIG. 3B a schematic side sectional view for explaining a structure of the shape measurement device, and illustrates a state in which the table 3 is moved into the electromagnetic shield device 2, and a heart magnetic field of the subject 6 is being measured. As illustrated in FIG. 3B, the Y-direction table 9 is moved in the +Y direction, and then the Z-direction table 11 is moved up. A distance by which the Z-direction table 11 is moved up is calculated by the controller 21.

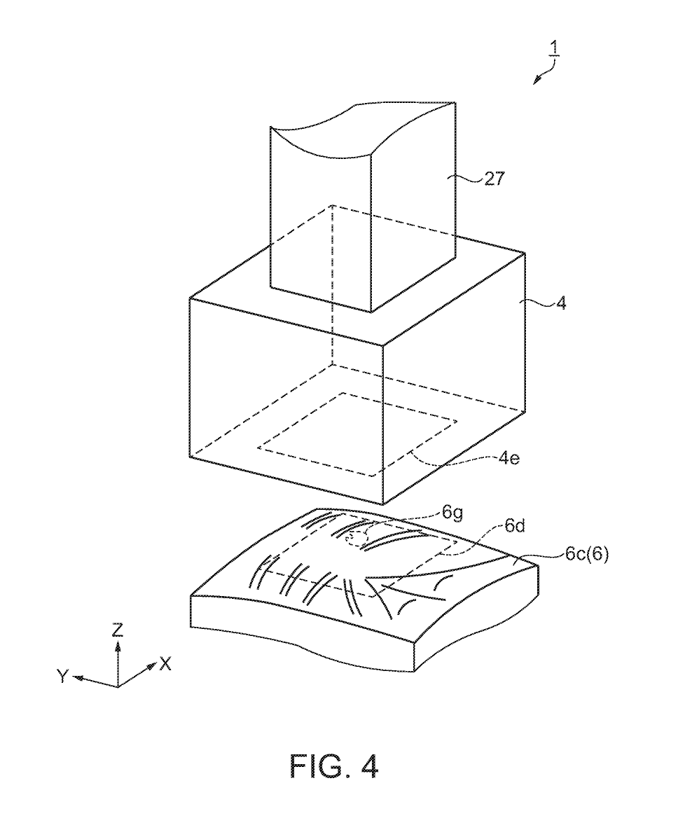

FIG. 4 is a main portion schematic perspective view illustrating a positional relationship between a measurement surface and the magnetic sensor. As illustrated in FIG. 4, a location measured by the magnetic sensor 4 on a surface of the chest 6c of the subject 6 is a measured surface 6d as a measured portion. In this case, the measured surface 6d is located at a location opposing the magnetic sensor 4, and comes close to the magnetic sensor 4. A surface which opposes the measured surface 6d in the magnetic sensor 4 is referred to as an opposing surface 4e or a first surface 4e. The controller 21 controls the table 3 so that the distance between the measured surface 6d and the magnetic sensor 4 becomes 5 mm as a predetermined distance. The measured surface 6d is measured by the magnetic sensor 4. The measured surface 6d is a surface opposing the heart 6g, and the magnetic sensor 4 detects a magnetic field generated from the heart 6g. A magnetic field generated due to an activity of the heart 6g is output from the surface of the chest 6c. As a result, the magnetic sensor 4 can detect the activity of the heart 6g.

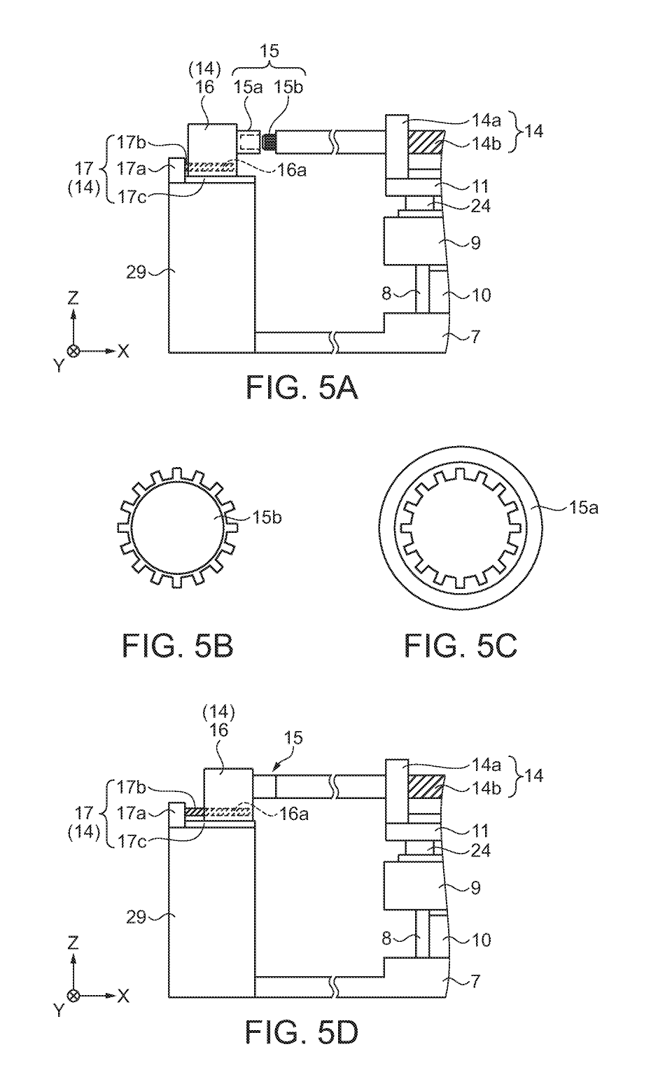

FIG. 5A is a schematic side view illustrating a structure of the attachment/detachment portion, and illustrates a separation state in which the attachment/detachment portion 15 is separated. As illustrated in FIG. 5A, an attachment/detachment portion installation stand 29 is on the −X direction side of the foundation 7. The motor movement portion 17 is provided at an end on the −X direction side on the attachment/detachment portion installation stand 29. The motor movement portion 17 is constituted of a motor 17a, a screw rod 17b, guide rails 17c, and the like. The motor 17a is provided on the −X direction side of the attachment/detachment portion installation stand 29, and the guide rails 17c are provided on the +X direction side of the motor 17a. The guide rails 17c are provided as a pair, and extend in the X direction.

The X-direction table motor 16 is provided on the guide rails 17c, and the X-direction table motor 16 is reciprocally moved along the guide rails 17c. A rotation shaft of the motor 17a is provided with the screw rod 17b extending in the X direction. A penetration hole 16a extending in the X direction is provided in the X-direction table motor 16, and a female screw is formed on the penetration hole 16a. The female screw of the penetration hole 16a is screwed with the screw rod 17b. When the motor 17a rotates the screw rod 17b, the X-direction table motor 16 is moved in the X direction along the guide rails 17c. A rotation shaft of the X-direction table motor 16 is provided with a grooved cylinder 15a. A grooved rod 15b is provided at an end on the −X direction side of the first screw rod 14b. When the X-direction table motor 16 is moved in the X direction, the grooved rod 15b is inserted into the grooved cylinder 15a.

FIG. 5B is a side view of the grooved rod, and is a view in which the grooved rod 15b is viewed from an axial direction. As illustrated in FIG. 5B, grooves are provided on an outer circumference of the grooved rod 15b. FIG. 5C is a side view of the grooved cylinder, and is a view in which the grooved cylinder 15a is viewed from an axial direction. As illustrated in FIG. 5C, grooves extending in the axial direction are provided on an inner diameter of the grooved cylinder 15a. An outer circumference shape of the grooved rod 15b is substantially the same as an inner circumference shape of the grooved cylinder 15a. When the grooved rod 15b is inserted into the grooved cylinder 15a, the grooves of the grooved cylinder 15a mesh with the grooves of the grooved rod 15b. Consequently, the torque applied to the grooved cylinder 15a is transmitted to the grooved rod 15b.

FIG. 5D is a schematic side view illustrating a structure of the attachment/detachment portion, and illustrates a connection state of the attachment/detachment portion 15. In FIG. 5D, the motor movement portion 17 moves the X-direction table motor 16 in the X direction, and thus the grooved rod 15b is inserted into the grooved cylinder 15a. When the X-direction table motor 16 rotates the rotation shaft, the grooved rod 15b is rotated due to rotation of the grooved cylinder 15a. Therefore, when the X-direction table motor 16 rotates the rotation shaft, the first screw rod 14b coupled to the grooved rod 15b is rotated. The X-direction table motor 16 moves the X-direction table 13 in the X direction.

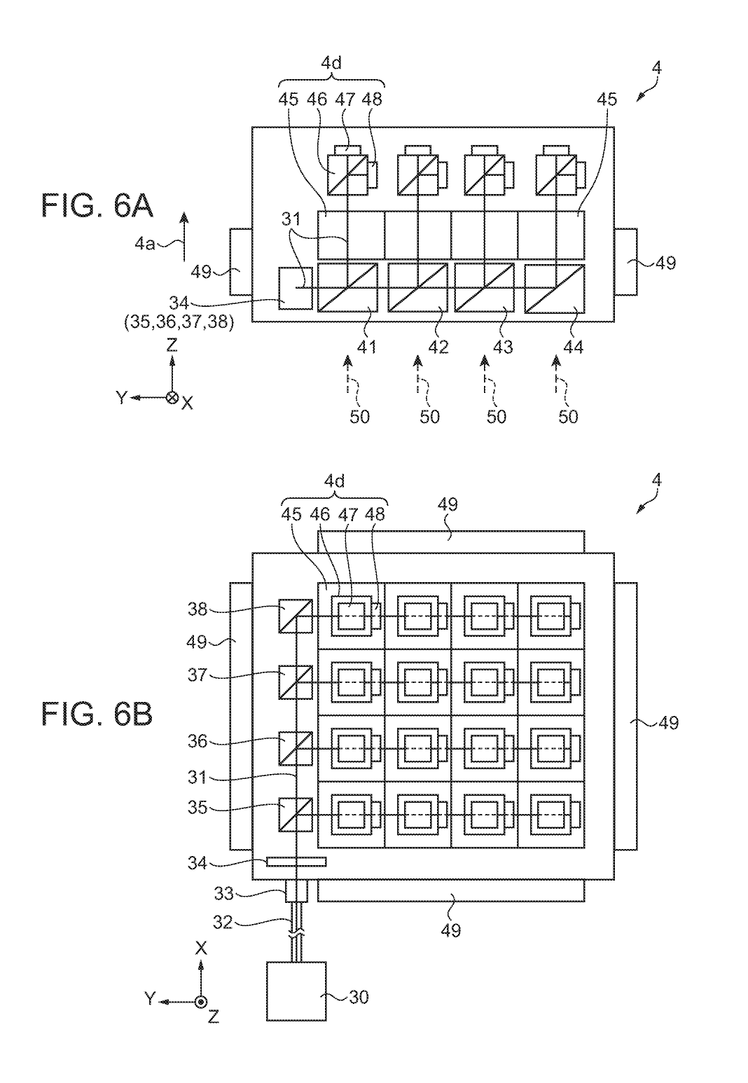

FIG. 6A is a schematic side view illustrating a structure of the magnetic sensor, and FIG. 6B is a schematic plan view illustrating a structure of the magnetic sensor. As illustrated in FIGS. 6A and 6B, laser light 31 is supplied from a laser light source 30 to the magnetic sensor 4. The laser light source 30 is provided in the controller 21, and the laser light 31 is supplied to the magnetic sensor 4 through an optical fiber 32. The magnetic sensor 4 is coupled to the optical fiber 32 via an optical connector 33.

The laser light source 30 outputs the laser light 31 with a wavelength corresponding to an absorption line of cesium. A wavelength of the laser light 31 is not particularly limited, but is set to a wavelength of 894 nm corresponding to the D1-line, in the present embodiment. The laser light source 30 is a tunable laser device, and the laser light 31 output from the laser light source 30 is continuous light with a predetermined light amount.

The laser light 31 supplied via the optical connector 33 travels in the +X direction and is applied to a polarization plate 34. The laser light 31 having passed through the polarization plate 34 is linearly polarized. The laser light 31 is sequentially applied to a first half mirror 35, a second half mirror 36, a third half mirror 37, and a first reflection mirror 38. Some of the laser light 31 is reflected by the first half mirror 35, the second half mirror 36, and the third half mirror 37 so as to travel in the −Y direction. The other light is transmitted therethrough so as to travel in the +X direction. The first reflection mirror 38 reflects the entire incident laser light 31 in the −Y direction. An optical path of the laser light 31 is divided into four optical paths by the first half mirror 35, the second half mirror 36, the third half mirror 37, and the first reflection mirror 38. Reflectance of each mirror is set so that light intensities of the laser light beams 31 on the respective optical paths are the same as each other.

Next, the laser light 31 is sequentially applied to a fourth half mirror 41, a fifth half mirror 42, a sixth half mirror 43, and a second reflection mirror 44. Some of the laser light 31 is reflected by the fourth half mirror 41, the fifth half mirror 42, and the sixth half mirror 43 so as to travel in the +Z direction. The other light 31 is transmitted therethrough so as to travel in the −Y direction. The second reflection mirror 44 reflects the entire incident laser light 31 in the +Z direction. A single optical path of the laser light 31 is divided into four optical paths by the fourth half mirror 41, the fifth half mirror 42, the sixth half mirror 43, and the second reflection mirror 44. Reflectance of each mirror is set so that light intensities of the laser light beams 31 on the respective optical paths are the same as each other. Therefore, the optical path of the laser light 31 is divided into the sixteen optical paths. In addition, reflectance of each mirror is set so that light intensities of the laser light beams 31 on the respective optical paths are the same as each other.

Gas cells 45 are provided on the respective optical paths of the laser light 31 on the +Z direction side of the fourth half mirror 41, the fifth half mirror 42, the sixth half mirror 43, and the second reflection mirror 44. The number of gas cells 45 is 16 in four rows and four columns. The laser light beams 31 reflected by the fourth half mirror 41, the fifth half mirror 42, the sixth half mirror 43, and the second reflection mirror 44 pass through the gas cells 45. The gas cell 45 is a box having a cavity therein, and an alkali metal gas is enclosed in the cavity. The alkali metal is not particularly limited, and potassium, rubidium, or cesium may be used. In the present embodiment, for example, cesium is used as the alkali metal.

A polarization separator 46 is provided on the +Z direction side of each gas cell 45. The polarization separator 46 is an element which separates the incident laser light 31 into two polarization components of the laser light 31, which are orthogonal to each other. As the polarization separator 46, for example, a Wollaston prism or a polarized beam splitter may be used.

A first photodetector 47 is provided on the +Z direction side of the polarization separator 46, and a second photodetector 48 is provided on the −Y direction side of the polarization separator 46. The laser light 31 having passed through the polarization separator 46 is applied to the first photodetector 47, and the laser light 31 reflected by the polarization separator 46 is applied to the second photodetector 48. The first photodetector 47 and the second photodetector 48 output signals corresponding to an amount of incident laser light 31 to the controller 21. If the first photodetector 47 and the second photodetector 48 generate magnetic fields, this may influence measurement, and thus it is preferable that the first photodetector 47 and the second photodetector 48 are made of a non-magnetic material. The magnetic sensor 4 includes heaters 49 which are provided on both sides in the X direction and both sides in the Y direction. Each of the heaters 49 preferably has a structure in which a magnetic field is not generated, and may employ, for example, a heater of a type of performing heating by causing steam or hot air to pass through a flow passage. Instead of using a heater, the gas cell 45 may be inductively heated by using a high frequency voltage.

The magnetic sensor 4 is disposed on the +Z direction side of the subject 6. A magnetic vector 50 as a magnetic field generated from the subject 6 enters the magnetic sensor 4 from the −Z direction side. The magnetic vector 50 passes through the fourth half mirror 41 to the second reflection mirror 44, and then passes through the gas cell 45. The magnetic vector 50 passes through the polarization separator 46, and comes out of the magnetic sensor 4.

The magnetic sensor 4 is a sensor which is called an optical pumping type magnetic sensor or an optical pumping atom magnetic sensor. Cesium in the gas cell 45 is heated and is brought into a gaseous state. The cesium gas is irradiated with the linearly polarized laser light 31, and thus cesium atoms are excited so that orientations of magnetic moments can be aligned. When the magnetic vector 50 passes through the gas cell 45 in this state, the magnetic moments of the cesium atoms precess due to a magnetic field of the magnetic vector 50. This precession is referred to as Larmore precession. The magnitude of the Larmore precession has a positive correlation with the strength of the magnetic vector 50. In the Larmore precession, a polarization plane of the laser light 31 is rotated. The magnitude of the Larmore precession has a positive correlation with a change amount of a rotation angle of the polarization plane of the laser light 31. Therefore, the strength of the magnetic vector 50 has a positive correlation with the change amount of a rotation angel of the polarization plane of the laser light 31. The magnetic sensor 4 has high sensitivity for a component of the magnetic vector 50 in the first direction 4a, and has low sensitivity for a component thereof orthogonal to the first direction 4a.

The polarization separator 46 separates the laser light 31 into two components of linearly polarized light which are orthogonal to each other. The first photodetector 47 and the second photodetector 48 respectively detect the strengths of the two components of linearly polarized light orthogonal to each other. Consequently, the first photodetector 47 and the second photodetector 48 can detect a rotation angle of a polarization plane of the laser light 31. The magnetic sensor 4 can detect strength of the magnetic vector 50 on the basis of a change of the rotation angel of the polarization plane of the laser light 31. An element constituted of the gas cell 45, the polarization separator 46, the first photodetector 47, and the second photodetector 48 is referred to as a sensor element 4d. In the present embodiment, sixteen sensor elements 4d of four rows and four columns are disposed in the magnetic sensor 4. The number and arrangement of the sensor elements 4d in the magnetic sensor 4 are not particularly limited. The sensor elements 4d may be disposed in three or less rows or five or more rows. Similarly, the sensor elements 4d may be disposed in three or less columns or five or more columns. The larger the number of sensor elements 4d, the higher the spatial resolution.

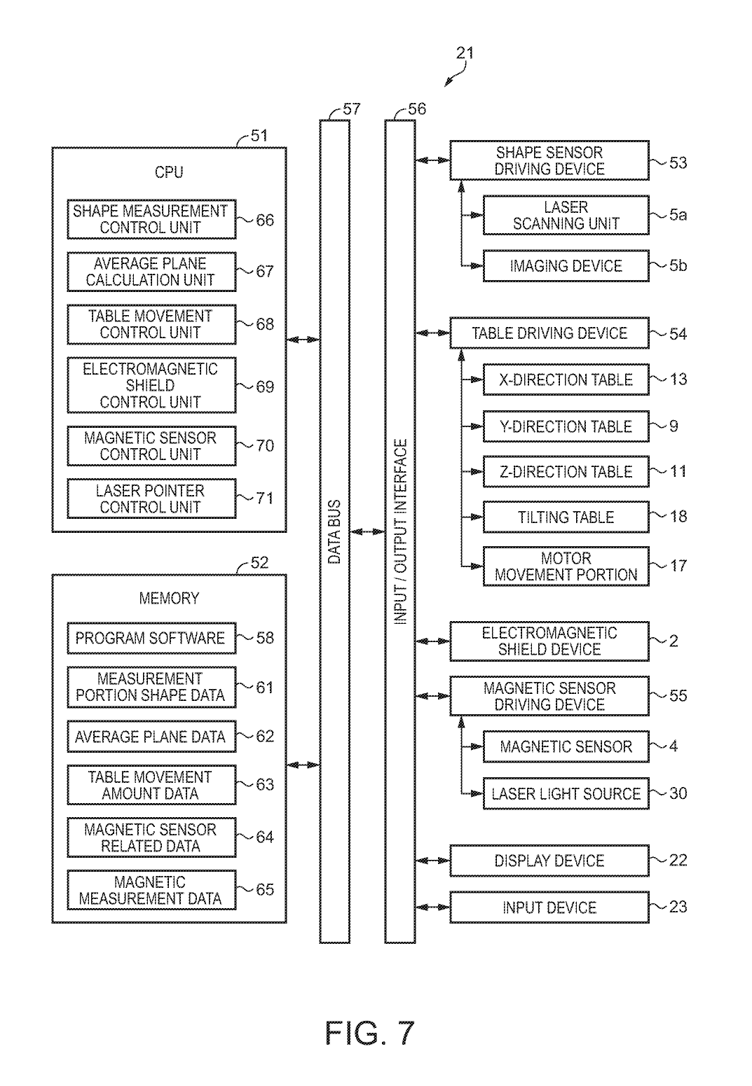

FIG. 7 is an electrical control block diagram of the controller. As illustrated in FIG. 7, the magnetic field measurement apparatus 1 includes the controller 21 controlling an operation of the magnetic field measurement apparatus 1. The controller 21 includes a central processing unit (CPU) 51 which performs various calculation processes as a processor, and a memory 52 which stores various information. A shape sensor driving device 53, a table driving device 54, the electromagnetic shield device 2, a magnetic sensor driving device 55, the display device 22, and the input device 23 are coupled to the CPU 51 via an input/output interface 56 and a data bus 57.

The shape sensor driving device 53 is a device which drives the laser scanning unit 5a and the imaging device 5b. The shape sensor driving device 53 drives the laser scanning unit 5a to emit the laser light 5c toward the subject 6. The shape sensor driving device 53 performs scanning with the laser light 5c in the horizontal direction. The shape sensor driving device 53 drives the imaging device 5b to capture an image of the reflection point 5d. In addition, the shape sensor driving device 53 irradiates a single location without scanning with the laser light 5c. The irradiated reflection point 5d is a guiding mark indicating a location where the subject 6 is positioned.

The table driving device 54 is a device which drives the X-direction table 13, the Y-direction table 9, the Z-direction table 11, the tilting table 18, and the motor movement portion 17. The table driving device 54 receives an instruction signal for moving a position of the X-direction table 13 from the CPU 51. The X-direction table 13 can be moved only when the Y-direction table 9 is located at a predetermined position. For this reason, first, the Y-direction table 9 is moved to the predetermined position. The table driving device 54 detects a position of the Y-direction table 9. The Y-direction table 9 includes a length measurement device detecting a position thereof, and the length measurement device detects a position of the Y-direction table 9. The table driving device 54 moves the Y-direction table 9, and thus the Y-direction table 9 is moved to a location where the grooved rod 15b opposes the grooved cylinder 15a.

Next, the table driving device 54 drives the motor movement portion 17 so that the grooved cylinder 15a is combined with the grooved rod 15b. Successively, the table driving device 54 detects a position of the X-direction table 13. The X-direction table 13 includes a length measurement device detecting a position thereof, and the length measurement device detects a position of the X-direction table 13. A difference between a position to which the X-direction table 13 is scheduled to be moved and the present position of the X-direction table 13 is calculated. The table driving device 54 drives the X-direction table motor 16 to move the X-direction table 13 to the position to which the X-direction table 13 is scheduled to be moved. Consequently, the table driving device 54 can move the X-direction table 13 to the location for which an instruction is given. Successively, the table driving device 54 drives the motor movement portion 17 to separate the grooved cylinder 15a from the grooved rod 15b.

Similarly, the table driving device 54 receives an instruction signal for moving a position of the Y-direction table 9 from the CPU 51. The table driving device 54 detects a position of the Y-direction table 9. A difference between a position to which the Y-direction table 9 is scheduled to be moved and the present position of the Y-direction table 9 is calculated. The table driving device 54 drives the motor 10a to move the Y-direction table 9 to the position to which the Y-direction table 9 is scheduled to be moved. Consequently, the table driving device 54 can move the Y-direction table 9 between the position inside the electromagnetic shield device 2 and the position outside the electromagnetic shield device 2. In a case where the shape measurement device 5 measures the chest 6c of the subject 6, the Y-direction table 9 is moved at a constant speed.

Similarly, the table driving device 54 receives an instruction signal for moving a position of the Z-direction table 11 from the CPU 51. Each of the lifting devices 24 lifting the Z-direction table 11 includes a length measurement device detecting position of the Z-direction table 11, and the table driving device 54 detects a position of the Z-direction table 11. A difference between a position to which the Z-direction table 11 is scheduled to be moved and the present position of the Z-direction table 11 is calculated. The lifting device 24 is an air cylinder, and the table driving device 54 is provided with pneumatic equipment such as a compressor or an electromagnetic valve driving the lifting device 24. The table driving device 54 controls an amount of air supplied to the lifting device 24 so as to move the Z-direction table 11 to the position to which the Z-direction table 11 is scheduled to be moved.

Similarly, the table driving device 54 receives an instruction signal for tilting the tilting table 18 from the CPU 51. The tilting device 26 tilting the tilting table 18 includes a length measurement device detecting a length of the tilting device 26. The table driving device 54 detects a tilt of the tilting table 18 by using the length of the tilting device 26 detected by the length measurement device. A difference between an angle at which the tilting table 18 is scheduled to be tilted and the present angle of the tilting table 18 is calculated. The tilting device 26 is an air cylinder, and the table driving device 54 is provided with pneumatic equipment such as a compressor or an electromagnetic valve driving the tilting device 26. The table driving device 54 controls an amount of air supplied to the tilting device 26 so as to tilt the tilting table 18 up to the angle at which the tilting table 18 is scheduled to be tilted.

The electromagnetic shield device 2 includes the first Helmholtz coil 2c and a sensor detecting an internal magnetic field. The electromagnetic shield device 2 reduces an internal magnetic field of the main body 2a by driving the first Helmholtz coil 2c in response to an instruction from the CPU 51.

The magnetic sensor driving device 55 is a device driving the magnetic sensor 4 and the laser light source 30. The magnetic sensor 4 is provided with the first photodetector 47, the second photodetector 48, and the heater 49. The magnetic sensor driving device 55 drives the laser light source 30, the heater 49, the first photodetector 47, and the second photodetector 48. The magnetic sensor driving device 55 drives the laser light source 30 to supply the laser light 31 to the magnetic sensor 4. The magnetic sensor driving device 55 drives the heater 49 so that the magnetic sensor 4 is maintained at a predetermined temperature. The magnetic sensor driving device 55 converts electric signals output from the first photodetector 47 and the second photodetector 48 into digital signals which are then output to the CPU 51.

The display device 22 displays predetermined information in response to an instruction from the CPU 51. The operator operates the input device 23 on the basis of the display content and inputs the instruction content. The instruction content is transmitted to the CPU 51.

The memory 52 is a concept including a semiconductor memory such as a RAM or a ROM, a hard disk, and an external storage device such as a DVD-ROM. In terms of a function, a storage region for storing program software 58 in which control procedures of an operation of the magnetic field measurement apparatus 1 are described, or a storage region for storing measurement portion shape data 61 which is data obtained by measuring a stereoscopic shape of the measured surface 6d of the subject 6 is set. In addition, a storage region for storing average plane data 62 obtained by calculating an average plane of the stereoscopic shape of the measured surface 6d of the subject 6 on the basis of the measurement portion shape data 61 is set. The average plane is a plane passing through an average location of points on a surface of the stereoscopic shape. Further, a storage region for storing table movement amount data 63 which is data regarding movement amounts of the X-direction table 13, the Y-direction table 9, and the Z-direction table 11, and a tilt angle of the tilting table 18, is set.

Still further, a storage region for storing magnetic sensor related data 64 which is data such as parameters used to drive the magnetic sensor 4 is set in the memory 52. Furthermore, a storage region for storing magnetic measurement data 65 which is data obtained by the magnetic sensor 4 measuring the measured surface 6d is set in the memory 52. Moreover, a storage region functioning as a work area for the CPU 51, a temporary file, or the like, and other various storage regions are set.

The CPU 51 controls measurement of a magnetic field generated from the heart of the subject 6 according to the program software 58 stored in the memory 52. As a specific function realizing unit, the CPU 51 includes a shape measurement control unit 66 which is a measurement unit. The shape measurement control unit 66 controls measurement of a stereoscopic shape of the measured surface 6d of the subject 6 by driving the shape measurement device 5 and the Y-direction table 9. The CPU 51 includes an average plane calculation unit 67 as a calculation unit. The average plane calculation unit 67 calculates an average plane by using a measurement result of the stereoscopic shape of the subject 6.

The CPU 51 includes a table movement control unit 68 as a control unit. The table movement control unit 68 controls movement and stoppage positions of the X-direction table 13, the Y-direction table 9, the Z-direction table 11, and the tilting table 18. The CPU 51 includes an electromagnetic shield control unit 69. The electromagnetic shield control unit 69 performs control for minimizing a magnetic field around the magnetic sensor 4 by driving the electromagnetic shield device 2.

The CPU 51 includes a magnetic sensor control unit 70. The magnetic sensor control unit 70 performs control for causing the magnetic sensor driving device 55 to drive the magnetic sensor 4 to detect the strength of the magnetic vector 50. The CPU 51 includes a laser pointer control unit 71. The laser pointer control unit 71 performs control for driving the laser scanning unit 5a to apply the laser light 5c to only a single point of a predetermined location.

In the present embodiment, the above-described respective functions of the magnetic field measurement apparatus 1 are realized in the program software by using the CPU 51, but, in a case where the above-described respective functions can be realized by hardware such as a stand-alone electronic circuit without using the CPU 51, such an electronic circuit may be used.

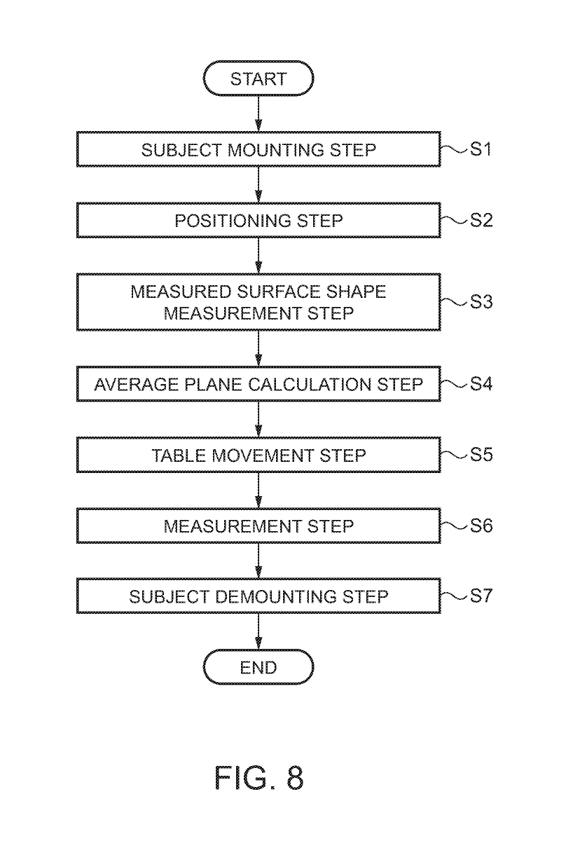

Next, a description will be made of a magnetic field measurement method using the above-described magnetic field measurement apparatus 1 with reference to FIGS. 8 to 12C. FIG. 8 is a flowchart illustrating a magnetic field measurement method. In the flowchart illustrated in FIG. 8, step S1 is a subject mounting step. In this step, the subject 6 is mounted on the tilting table 18. Next, the flow proceeds to step S2. Step S2 is a positioning step. In this step, the laser scanning unit 5a irradiates one location of the chest 6c with the laser light 5c. In this step, the operator operates the input device 23 so that the X-direction table 13 and the Y-direction table 9 are moved, and thus the xiphisternum 6e of the subject 6 is irradiated with the reflection point 5d. Next, the flow proceeds to step S3.

Step S3 corresponds to a measured surface shape measurement step. In this step, the shape measurement control unit 66 drives the Y-direction table 9 and the shape measurement device 5 to measure a surface shape of the measured surface 6d of the subject 6. Next, the flow proceeds to step S4. Step S4 is an average plane calculation step. In this step, the average plane calculation unit 67 calculates an average plane by using data regarding the measured surface shape. Next, the flow proceeds to step S5.

Step S5 is a table movement step. In this step, the table movement control unit 68 moves the table 3 and tilts the tilting table 18 so that the average plane of the measured surface 6d becomes parallel to the opposing surface 4e. The chest 6c of the subject 6 is moved to a location opposing the magnetic sensor 4. The measured surface 6d of the subject 6 comes close to the magnetic sensor 4. Next, the flow proceeds to step S6. Step S6 is a measurement step. In this step, the magnetic sensor control unit 70 causes the magnetic sensor driving device 55 to drive the magnetic sensor 4. The magnetic sensor 4 detects a magnetic field coming out of the chest 6c of the subject 6. Next, the flow proceeds to step S7. Step S7 is a subject demounting step. In this step, the table 3 is moved out of the electromagnetic shield device 2, and the subject 6 leaves the tilting table 18. Through the above steps, the process of measuring a magnetic field of the subject 6 is finished.

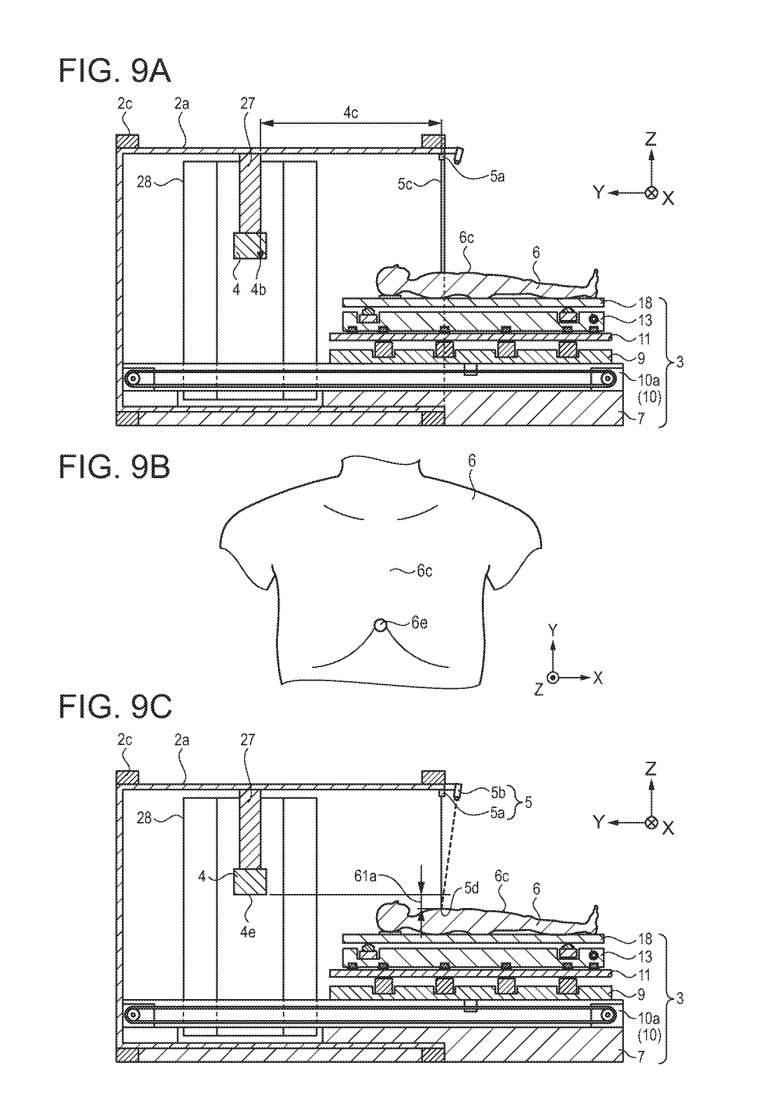

Next, with reference to FIGS. 9A to 12C, the magnetic field measurement method will be described in more detail so as to correspond to the steps illustrated in FIG. 8. FIGS. 9A to 12C are schematic diagrams for explaining the magnetic field measurement method. FIG. 9A is a diagram corresponding to the subject mounting step of step S1. As illustrated in FIG. 9A, in step S1, the subject 6 is mounted on the tilting table 18. A half or more of the tilting table 18 protrudes out of the electromagnetic shield device 2. The Z-direction table 11 is located at a low position, and thus the subject 6 easily moves onto the tilting table 18.

FIGS. 9A and 9B are diagrams corresponding to the positioning step of step S2. As illustrated in FIG. 9A, in step S2, the operator operates the input device 23 so as to input an instruction for starting positioning. The laser pointer control unit 71 outputs an instruction signal for applying the laser light 5c, to the shape sensor driving device 53. The shape sensor driving device 53 receives the instruction signal so as to drive the laser scanning unit 5a. The laser scanning unit 5a performs irradiation with the laser light 5c in the −Z direction. The laser light 5c is applied to a single point located in the −Z direction from the laser scanning unit 5a.

As illustrated in FIG. 9B, the xiphisternum 6e is present on the −Y direction side of the chest 6c in the subject 6. The xiphisternum 6e is a protrusion which protrudes at the lower end of sternum, and is present at a part called the pit of the stomach at which the rib bows join together. Referring to FIG. 9A again, the operator operates the input device 23 so as to input an instruction for moving the X-direction table 13 in the X direction. The table movement control unit 68 outputs a signal for moving the X-direction table 13, to the table driving device 54. The table driving device 54 drives the motor movement portion 17 to move the X-direction table motor 16 in the +X direction. Consequently, the grooved cylinder 15a is connected to the grooved rod 15b.

Next, the table driving device 54 drives the X-direction table motor 16 to move the X-direction table 13 in the X direction. The X-direction table 13 is moved in tracking of an instruction which is input by the operator via the input device 23. The operator causes the Y direction side of the xiphisternum 6e to be irradiated with the laser light 5c.

Successively, the operator operates the input device 23 so as to input an instruction for moving the Y-direction table 9. The table movement control unit 68 outputs a signal for moving the Y-direction table 9, to the table driving device 54. The table driving device 54 drives the motor movement portion 17 to move the X-direction table motor 16 in the −X direction. Consequently, the grooved cylinder 15a is separated from the grooved rod 15b.

Next, the table driving device 54 drives the motor 10a to move the Y-direction table 9 in the Y direction. The Y-direction table 9 is moved in tracking of an instruction which is input by the operator via the input device 23. The operator causes the xiphisternum 6e to be irradiated with the laser light 5c. Thereafter, the operator operates the input device 23 so as to input information indicating that positioning of the subject 6 has been finished.

A reference point 4b for checking a measurement point is set in the magnetic sensor 4. A position of the reference point 4b in the X direction is the same as the position in the X direction where the laser light 5c is applied in step S2. A distance in the Y direction between the position of the reference point 4b and a position through which the laser light 5c passes is set to a predetermined reference distance 4c.

FIG. 9C is a diagram corresponding to the measured surface measurement step of step S3. In step S3, the operator causes the subject 6 to take a normal breath. The subject 6 may take a deep breath so as to control his or her breathing. The operator operates the input device 23 so as to input an instruction for starting measurement of a stereoscopic shape of the measured surface 6d. The shape measurement control unit 66 receives the instruction for starting measurement, and outputs an instruction signal for applying the laser light 5c, to the shape sensor driving device 53. As illustrated in FIG. 9C, the laser scanning unit 5a irradiates the measured surface 6d with the laser light 5c, and reciprocally moves the reflection point 5d in the X direction. The imaging device 5b receives the reflected light 5f. Since the reflection point 5d is reciprocally moved on the measured surface 6d, the imaging device 5b captures an image in which the reflection point 5d forms a line. The measured surface 6d is uneven, and the image is an image of a curve. The shape sensor driving device 53 calculates a distance from the laser scanning unit 5a to the reflection point 5d by using the image data and a triangulation method, and outputs the calculated distance to the memory 52. The memory 52 stores data regarding the distance from the laser scanning unit 5a to the reflection point 5d as a part of the measurement portion shape data 61.

The shape measurement control unit 66 outputs an instruction signal for moving the Y-direction table 9 to the table driving device 54 in cooperation with the table movement control unit 68. A movement range of the Y-direction table 9 is the same as a range of the measured surface 6d. The table driving device 54 moves the Y-direction table 9 in the −Y direction and then moves the Y-direction table 9 in the +Y direction at a predetermined speed. The table driving device 54 outputs data indicating a position of the Y-direction table 9 in the Y direction to the memory 52. Consequently, the measurement portion shape data 61 of the memory 52 accumulates data regarding the distance between the laser scanning unit 5a and the reflection point 5d on the measured surface 6d. Next, the shape measurement control unit 66 subtracts a value of the distance between the shape measurement device 5 and the opposing surface 4e from the measurement portion shape data 61. Consequently the measurement portion shape data 61 becomes data regarding a distance 61a between the opposing surface 4e and the chest 6c.

When the shape measurement device 5 completes the measurement within the range of the measured surface 6d, the table movement control unit 68 outputs an instruction signal for moving the Y-direction table 9 to the table driving device 54 so that the xiphisternum 6e is located at the location opposing the laser scanning unit 5a. The table driving device 54 receives the instruction signal and moves the Y-direction table 9. The operator gives a message that the subject 6 is allowed to take a deep breath.

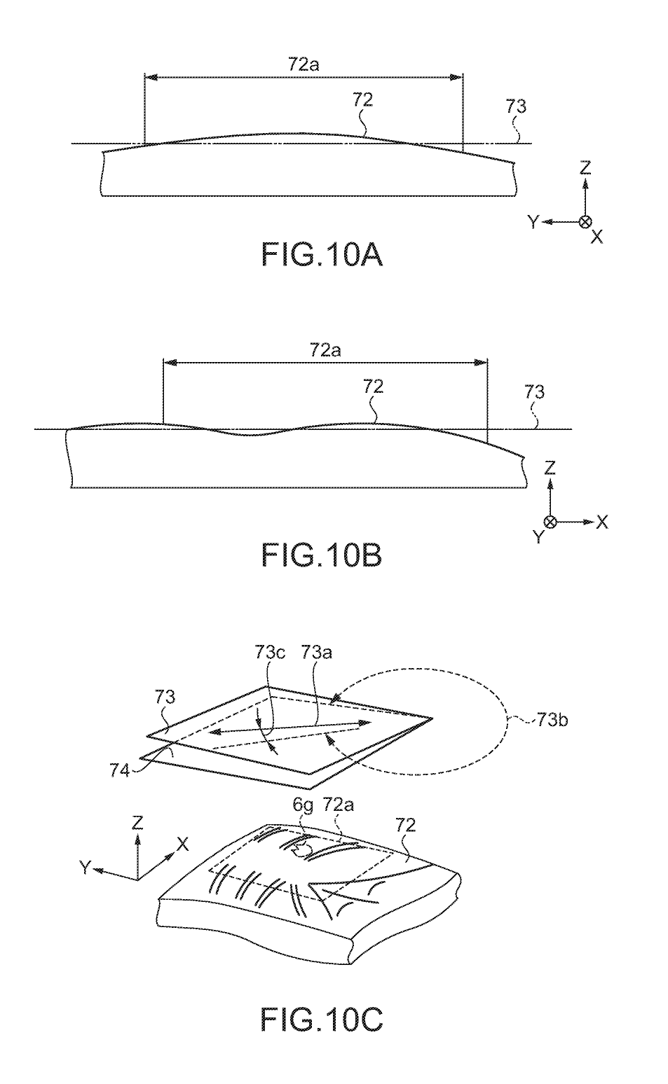

FIGS. 10A to 10C are diagrams corresponding to the average plane calculation step of step S4. As illustrated in FIGS. 10A and 10B, in step S4, the average plane calculation unit 67 calculates an average plane on the measured surface 6d. The measurement portion shape data 61 is data regarding a combination of an X coordinate and a Y coordinate of each measurement point, and the distance 61a. A measured surface 72 in the FIGS. 10A to 10C represents a shape obtained by plotting the measurement portion shape data 61. A measurement range 72a indicates a range of the measured surface 6d where a magnetic field is measured. The measured surface 72 is uneven in the measurement range 72a. The average plane calculation unit 67 calculates coefficients of an equation representing an average plane 73 by using a least square method and an approximation method. Specifically, in an equation of aX+bY+cZ+d=0, the coefficients a, b, c and d are calculated.

FIG. 10C is a diagram illustrating that the average plane 73 is moved from the measured surface 72 in the Z direction. In FIG. 10C, the average plane 73 and a horizontal plane 74 are illustrated. The horizontal plane 74 corresponds to an initial state of the tilting table 18, and indicates an upper surface of the tilting table 18 when tilting is not performed. The average plane calculation unit 67 calculates a tilt direction 73a of the average plane 73. The tilt direction 73a is a direction in which a tilt angle is the maximum. An angle of the tilt direction 73a on the horizontal plane 74 is referred to as a tilt direction azimuth angle 73b. An angle formed between the average plane 73 and the horizontal plane 74 in the tilt direction 73a is referred to as a tilt direction deviation angle 73c. The average plane calculation unit 67 calculates the tilt direction azimuth angle 73b and the tilt direction deviation angle 73c by using the equation representing the average plane 73.

Next, the average plane calculation unit 67 calculates an X-direction component and a Y-direction component of the tilt direction deviation angle 73c. Successively, an angle of the Y-direction component of the tilt direction deviation angle 73c is multiplied by a distance of a Y-direction component between the first tilting portion 26a and the second tilting portion 26b. Consequently, a difference between a distance by which the first tilting portion 26a is moved up and a distance by which the second tilting portion 26b is moved up is calculated. Next, an angle of the X-direction component of the tilt direction deviation angle 73c is multiplied by a distance of an X-direction component between the second tilting portion 26b and the third tilting portion 26c. Consequently, a difference between a distance by which the second tilting portion 26b is moved up and a distance by which the third tilting portion 26c is moved up is calculated. A rise distance of a portion which is not required to be moved up is set to 0 among the first tilting portion 26a to the third tilting portion 26c. Rise distances of the first tilting portion 26a to the third tilting portion 26c are calculated. The average plane calculation unit 67 calculates a location where the distance 61a between the opposing surface 4e and the measured surface 6d is shortest when the average plane 73 is set to be parallel to the horizontal plane 74, and the distance 61a.

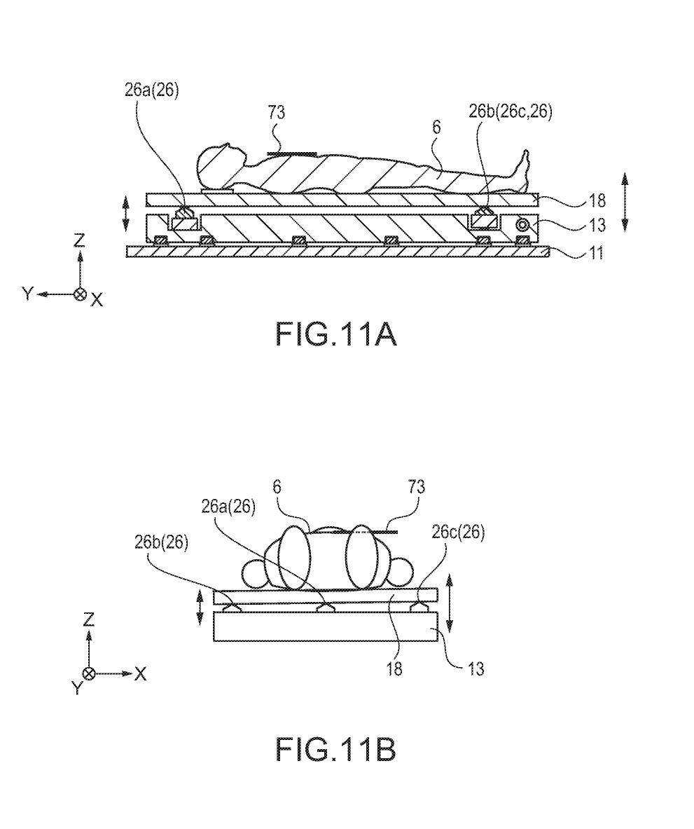

FIGS. 11A and 11B are diagrams corresponding to the table movement step of step S5. As illustrated in FIG. 11A, the table movement control unit 68 causes the table driving device 54 to tilt the tilting table 18. First, the tilting table 18 is tilted with the X direction as an axis. At this time, the table movement control unit 68 expands the second tilting portion 26b and the third tilting portion 26c by the same length, and expands the first tilting portion 26a. The first tilting portion 26a to the third tilting portion 26c are expanded so that a Y-direction component of an inclination of the tilt direction 73a becomes horizontal.

Next, as illustrated in FIG. 11B, the table movement control unit 68 tilts the tilting table 18 with the Y direction as an axis. At this time, the table movement control unit 68 expands one of the second tilting portion 26b and the third tilting portion 26c, and contracts the other. An X-direction component of the tilt direction deviation angle 73c is made horizontal. Consequently, the average plane 73 can be made parallel to the horizontal plane 74.

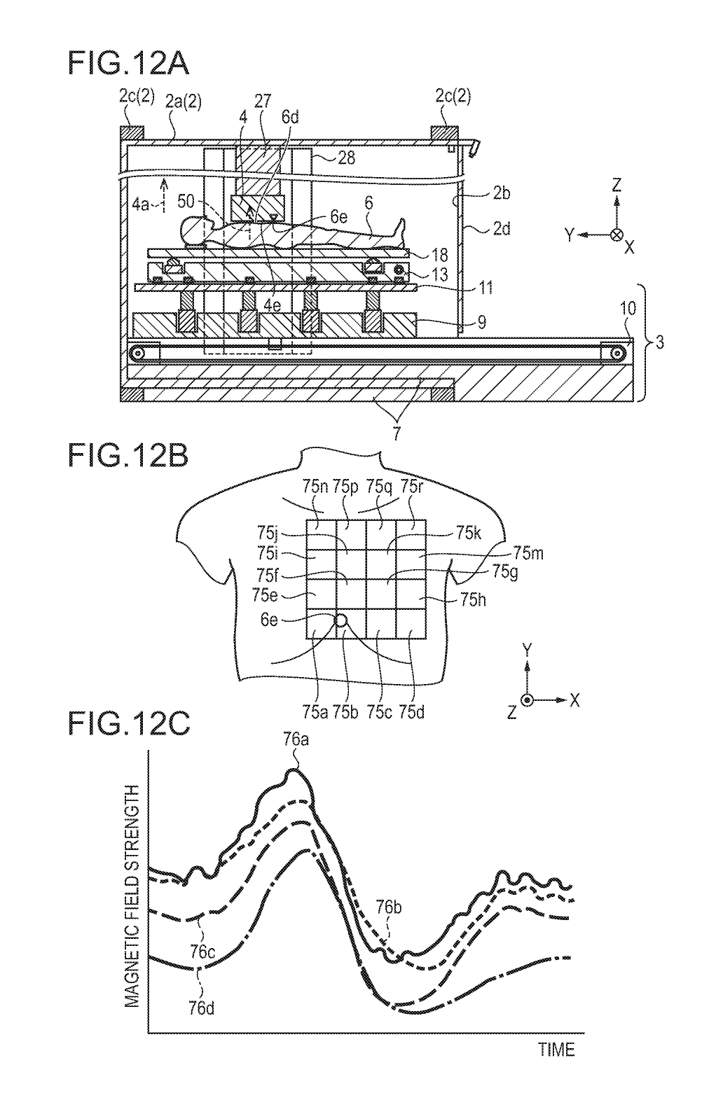

FIG. 12A is a diagram corresponding to the table movement step of step S5 and the measurement step of step S6. As illustrated in FIG. 12A, in step S5, the table movement control unit 68 outputs an instruction signal for moving the Y-direction table 9, to the table driving device 54. The table driving device 54 receives the instruction signal so as to move the Y-direction table 9 by the reference distance 4c in the +Y direction. As a result, the reference point 4b is located at a location opposing the xiphisternum 6e, and the measured surface 6d is located at a location opposing the magnetic sensor 4.

Next, the table movement control unit 68 outputs an instruction signal for moving up the Z-direction table 11 to the table driving device 54. The table driving device 54 receives the instruction signal so as to move up the Z-direction table 11 in the +Z direction. The Z-direction table 11 is moved up at the location where the distance between the measured surface 6d and the opposing surface 4e is shortest so that the distance becomes 5 mm. The distance between the measured surface 6d and the opposing surface 4e is not limited to 5 mm, and may be changed depending on a body shape of the subject 6. When the subject 6 takes a normal breath, a state occurs in which the opposing surface 4e of the magnetic sensor 4 is not in contact with the measured surface 6d. The distance between the measured surface 6d and the opposing surface 4e is short within a range in which the subject 6 is not in contact with the magnetic sensor 4. Since the magnetic sensor 4 vibrates when the measured surface 6d is in contact with the magnetic sensor 4, the measurement accuracy is reduced. In the present embodiment, since the subject 6 comes close to the magnetic sensor 4 within a range in which the measured surface 6d is not in contact with the magnetic sensor 4, the magnetic field measurement apparatus 1 can detect a magnetic field of the measured surface 6d with high accuracy.

If the magnetic sensor 4 becomes distant from the measured surface 6d, the strength of a magnetic field detected by the magnetic sensor 4 is in inverse proportion to the square of a distance from the measured surface 6d. Therefore, detection performance of the magnetic sensor 4 is reduced as the magnetic sensor 4 becomes more distant from the measured surface 6d. In the present embodiment, the measured surface 6d comes close to the magnetic sensor 4 to the extent to which the measured surface 6d is parallel to the opposing surface 4e, and is not in contact with the magnetic sensor 4. Therefore, the magnetic field measurement apparatus 1 can detect a magnetic field of the measured surface 6d with high accuracy. The table 3 is moved, and the door 2d is closed. Consequently, it is possible to prevent an external magnetic field from entering the electromagnetic shield device 2 through the opening 2b.

FIGS. 12A to 12C are diagrams corresponding to the measurement step of step S6. As illustrated in FIG. 12A, in step S6, the magnetic sensor 4 detects the magnetic vector 50 which travels in the first direction 4a from the measured surface 6d of the subject 6. The magnetic sensor control unit 70 outputs an instruction signal for starting measurement to the magnetic sensor driving device 55. The magnetic sensor driving device 55 receives the instruction signal for starting measurement, and drives the laser light source 30 and the heater 49. The laser light source 30 applies the laser light 31. If light emission of the laser light source 30 is stabilized, and the magnetic sensor 4 is stabilized at a predetermined temperature, the measurement is started. The strength of a magnetic field detected by the magnetic sensor 4 is output as an electric signal. The magnetic sensor driving device 55 converts electric signals output from the first photodetector 47 and the second photodetector 48 into electric signals indicating the strength of the magnetic field. The magnetic sensor driving device 55 converts the electric signals indicating the strength of the magnetic field into digital data which is then transmitted to the memory 52 as the magnetic measurement data 65.

In FIG. 12B, a first region 75a to a sixteenth region 75r indicate regions where the respective sensor elements 4d detect the magnetic vector 50. The first region 75a to the sixteenth region 75r are disposed in a lattice form of four rows and four columns. The xiphisternum 6e is disposed in the second region 75b. In this arrangement, the magnetic sensor 4 can detect the magnetic vector 50 generated from the heart of the subject 6 without leakage within the range of the first region 75a to the sixteenth region 75r.

FIG. 12C illustrates an example of change data of a magnetic field detected by the magnetic sensor 4. A longitudinal axis expresses the magnetic field strength, and the strength of an upper part in FIG. 12C is higher than the strength of a lower part therein. A transverse axis expresses a change in time, and time changes from a left part to a right part in FIG. 12C. The strength of the magnetic vector 50 detected by the sensor element 4d is referred to as the magnetic field strength. A first change line 76a indicates a change in the magnetic field strength in the twelfth region 75m, and indicates a change in the magnetic field strength on the upper left side of the heart. The upper left side of the heart represents a position in the X direction and the Y direction. A second change line 76b indicates a change in the magnetic field strength in the fourth region 75d, and indicates a change in the magnetic field strength on the lower left side of the heart. A third change line 76c indicates a change in the magnetic field strength in the second region 75b, and indicates a change in the magnetic field strength on the lower right side of the heart. A fourth change line 76d indicates a change in the magnetic field strength in the tenth region 75j, and indicates a change in the magnetic field strength on the upper right side of the heart. Sixteen magnetic field strength change lines can be obtained from the magnetic sensor 4. In FIG. 12C, for better viewing, four change lines are illustrated.

The first change line 76a has a peak, and then the second change line 76b has a peak. Next, the third change line 76c has a peak, and then the fourth change line 76d has a peak. As mentioned above, the peaks of the magnetic field strength move around the heart. When the heart does not normally operate, waveforms of the first change line 76a to the fourth change line 76d are deformed. Therefore, the operator can diagnose heart diseases of the subject 6 by observing waveforms of the first change line 76a to the fourth change line 76d.

After the measurement of a magnetic field is completed, the Z-direction table 11 is moved down, and the Y-direction table 9 is moved in the −Y direction, in the subject demounting step of step S7. The subject 6 leaves the table 3, and thus the process of measuring a magnetic field from the heart of the subject 6 is finished.

As described above, according to the present embodiment, the following effects are achieved.

(1) According to the present embodiment, the magnetic field measurement apparatus 1 includes the table 3, and the subject 6 is mounted on the table 3. The shape measurement device 5 measures a surface shape of the measured surface 6d of the subject 6. Next, the average plane calculation unit 67 calculates the average plane 73 of the surface shape of the subject 6. The measured surface 6d of the subject 6 is a curved surface, and the average plane calculation unit 67 sets the average plane 73 so that deviation with the measured surface 6d is minimized. Next, the table movement control unit 68 controls a tilt of the table 3 so that the opposing surface 4e is parallel to the average plane 73. The magnetic sensor 4 detects a magnetic field coming out of the subject 6 in a state in which the opposing surface 4e of the magnetic sensor 4 is parallel to the average plane 73, and the distance between the average plane 73 and the opposing surface 4e is short.

As the subject 6 and the opposing surface 4e become more distant from each other, a magnetic field reaching the opposing surface 4e becomes weaker, and thus a signal-to-noise ratio (S/N ratio) of a signal output from the magnetic sensor 4 is lowered. If the subject 6 is in contact with the opposing surface 4e, the magnetic sensor 4 receives vibration from the subject 6, and noise increases due to the vibration. In the present embodiment, the table movement control unit 68 can control a tilt of the table 3 so as to make the average plane 73 parallel to the opposing surface 4e of the magnetic sensor 4, and can cause the subject 6 to sufficiently come close to the opposing surface 4e in a range in which there is no contact therebetween. As a result, the magnetic sensor 4 can detect a magnetic field coming out of the subject 6 with high sensitivity.

(2) According to the present embodiment, the table movement control unit 68 controls the table 3 so that the distance between the opposing surface 4e and the subject 6 becomes a predetermined distance. The predetermined distance is longer than a distance which the subject 6 moves through a normal action of the subject 6 such as breathing. The predetermined distance is short within a range in which the subject 6 is not in contact with the magnetic sensor 4. The table movement control unit 68 controls the table 3 so that the distance between the subject 6 and the opposing surface 4e becomes a distance which does not cause contact therebetween due to an action of the subject 6. As a result, the subject 6 can be made to come close to the magnetic sensor 4 within a range in which the subject 6 is not in contact therewith.

(3) According to the present embodiment, the magnetic field measurement apparatus 1 includes the electromagnetic shield device 2. The electromagnetic shield device 2 attenuates an entering magnetic field line. The magnetic sensor 4 is provided inside the electromagnetic shield device 2 so as to measure a magnetic field. The electromagnetic shield device 2 includes the opening 2b and attenuates a magnetic field line coming through the opening 2b. Consequently, the electromagnetic shield device 2 can perform measurement with less noise. The shape measurement device 5 is provided in the opening 2b through which the subject 6 comes in and out. Since the subject 6 passes near the shape measurement device 5, the shape measurement device 5 can easily measure a shape of the measured surface 6d of the subject 6.

(4) According to the present embodiment, the shape measurement device 5 scans the subject 6 with a light beam. A location irradiated with the laser light 5c is measured. A surface shape of the subject 6 has an unevenness, and a position where the laser light 5c is reflected differs on the unevenness. Therefore, the shape measurement device 5 can easily detect a surface shape of the subject 6 by detecting a position of the laser light 5c reflected from the subject 6.

(5) According to the present embodiment, the shape measurement device 5 has a function of a guide light irradiation unit which applies the laser light 5c for guiding a position where the subject 6 is mounted on the table 3, and a function of measuring a shape of the subject 6. The xiphisternum 6e of the subject 6 is positioned at a location indicated by the laser light 5c, and thus the subject 6 can be mounted at a predetermined position on the table 3. The shape measurement device 5 also has a function of measuring a shape of the subject 6 by irradiating the subject 6 with a light beam. Therefore, it is possible to reduce the number of constituent elements compared with a case where the magnetic field measurement apparatus 1 separately includes the guide light irradiation unit and the shape measurement device 5. As a result, it is possible to manufacture the magnetic field measurement apparatus 1 with high productivity.

(6) According to the present embodiment, the tilting table 18 is provided with the tilting devices 26 including the three tilting portions. The table movement control unit 68 controls a length of the tilting device 26 so as to tilt the subject 6. Consequently, the average plane 73 of the subject 6 can be made parallel to the opposing surface 4e. The table 3 may have a structure in which a device controlling a tilt is provided at the center thereof. In contrast to this structure, loads of the table 3 and the subject 6 can be distributed to three leg portions, and thus it is possible to control a tilt of the table 3 with a lightweight structure.

(7) According to the present embodiment, a portion of the table 3 which is moved into the electromagnetic shield device 2 is non-magnetic. Therefore, it is possible to prevent magnetization of the table 3 from influencing measurement of a magnetic field.

(8) According to the present embodiment, a location where the magnetic sensor 4 detects a magnetic field is a surface of the chest 6c opposing the heart 6g. A magnetic field generated due to the activity of the heart 6g is output from the surface of the chest 6c. As a result, the magnetic sensor 4 can detect the activity of the heart 6g.

(9) According to the present embodiment, the subject 6 is mounted on the table 3, and a shape of the subject 6 is measured. The average plane 73 of the subject 6 is calculated. The measured surface 6d of the subject 6 is a curved surface, and the average plane 73 is set so that deviation with the measured surface 6d is minimized. Next, the table movement control unit 68 controls a tilt of the table 3 so that the opposing surface 4e of the magnetic sensor 4 is parallel to the average plane 73 of the subject 6. The subject 6 is made to come close to the opposing surface 4e of the magnetic sensor 4, and the magnetic sensor 4 detects the magnetic vector 50 coming out of the subject 6. In the present embodiment, the average plane 73 of the subject 6 is calculated. The table movement control unit 68 controls a tilt of the table 3 so that the average plane 73 is parallel to the opposing surface 4e of the magnetic sensor 4. The subject 6 is made to come close to the opposing surface 4e in a form in which the subject 6 hardly contacts the opposing surface 4e. As a result, the magnetic sensor 4 can detect the magnetic vector 50 coming out of the subject 6 with high sensitivity.

### Second Embodiment

In the present embodiment, with reference to the drawings, a description will be made of characteristic examples of a living body magnetic field measurement apparatus and a living body magnetic field measurement method of measuring a heart magnetic field generated from the heart by using the magnetic field measurement apparatus. A difference between the present embodiment and the first embodiment is that a shape of a table is deformed according to a shape of a back of a subject. A description of the same content as that in the first embodiment will not be repeated.

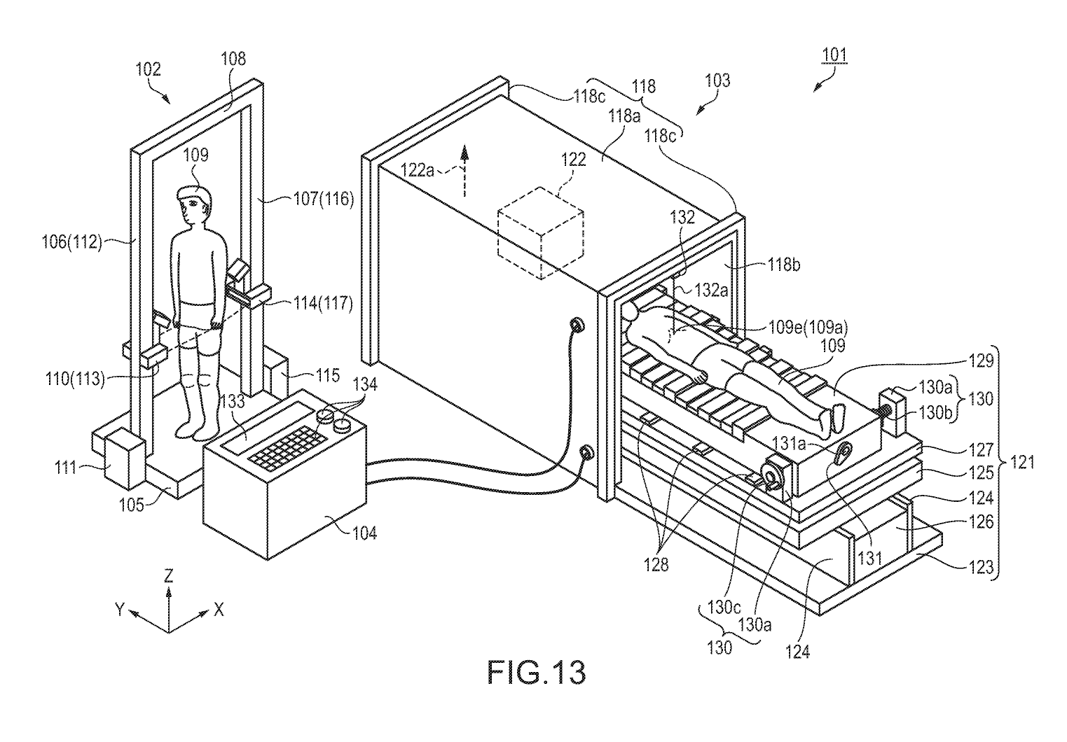

With reference to FIGS. 13 to 18, a description will be made of a structure of a living body magnetic field measurement apparatus according to the present embodiment. FIG. 13 is a schematic perspective view illustrating a configuration of the living body magnetic field measurement apparatus. As illustrated in FIG. 13, a living body magnetic field measurement apparatus 101 includes a contour measurement section 102 and a magnetic field measurement section 103 as a measurement unit, and a controller 104 controlling the contour measurement section 102 and the magnetic field measurement section 103.

The contour measurement section 102 includes a first foundation 105, and a first rail 106 and a second rail 107 are provided to be erect on the first foundation 105. A thickness direction of the first foundation 105 is set to a Z direction. The Z direction is a vertical direction. Directions in which an upper surface of the first foundation 105 extends are set to an X direction and a Y direction. The X direction and the Y direction are a horizontal direction, and the X direction and the Y direction are orthogonal to each other. The first rail 106 and the second rail 107 are provided side by side in the X direction. A beam portion 108 which crosslinks the first rail 106 with the second rail 107 is provided at ends on the Z direction sides of the first rail 106 and the second rail 107. The beam portion 108 can increase the strength of the first rail 106 and the second rail 107.

A subject 109 is disposed at an intermediate location between the first rail 106 and the second rail 107. The subject 109 stands on the first foundation 105 and faces the first rail 106. The contour measurement section 102 is a device which measures a surface shape or a contour of the subject 109. The first rail 106 is provided with a front stage 110, and the front stage 110 is movable in a lifting manner along the first rail 106. A first motor 111 is provided at the first rail 106 on the first foundation 105 side, and a first linear movement mechanism 112 is provided inside the first rail 106. The front stage 110 is lifted by the first motor 111 and the first linear movement mechanism 112. The first motor 111 is a stepping motor. The first linear movement mechanism 112 may employ a configuration in which a ball screw or a timing belt and a pulley are combined with each other. In the present embodiment, for example, the ball screw is used in the first linear movement mechanism 112. The front stage 110 is provided with a front sensor 113, and the front sensor 113 measures a surface shape of the chest of the subject 109.

Similarly, the second rail 107 is provided with a back stage 114, and the back stage 114 is movable in a lifting manner along the second rail 107. A second motor 115 is provided at the second rail 107 on the first foundation 105 side, and a second linear movement mechanism 116 is provided inside the second rail 107. The back stage 114 is lifted by the second motor 115 and the second linear movement mechanism 116. The same motor as the first motor 111 is used as the second motor 115. The same linear movement mechanism as the first linear movement mechanism 112 may be used as the second linear movement mechanism 116. A back sensor 117 is provided in the back stage 114, and the back sensor 117 measures a surface shape of a back of the subject 109 or the like.

The magnetic field measurement section 103 mainly includes an electromagnetic shield device 118 as a magnetic shield unit, a table 121, and a magnetic sensor 122 as a magnetic detection unit. The electromagnetic shield device 118 includes a rectangular tubular main body 118a so as to prevent a situation in which an external magnetic field such as terrestrial magnetism enters a space where the magnetic sensor 122 is disposed. In other words, the influence of the external magnetic field on the magnetic sensor 122 is minimized by the electromagnetic shield device 118, and a magnetic field in the location where the magnetic sensor 122 is present is considerably lower than the external magnetic field. The main body 118a extends in the Y direction, and thus functions as a passive magnetic shield. The inside of the main body 118a is hollow, and a sectional shape of surfaces (orthogonal planes in the Y direction in the XZ section) passing through the X direction and the Z direction is a substantially quadrangle shape. A sectional shape of the main body 118a is a square shape. The electromagnetic shield device 118 is provided with a first opening 118b on the −Y direction side, and the table 121 protrudes out of the first opening 118b. Regarding the size of the electromagnetic shield device 118, for example, a length thereof in the Y direction is about 200 cm, and one side of the first opening 118b is about 90 cm. The subject 109 laid down on the table 121 can come in and out of the electromagnetic shield device 118 via the first opening 118b along with the table 121.

The controller 104 is provided at a location separated from the first opening 118b. The controller 104 makes an electric signal flow so as to control the living body magnetic field measurement apparatus 101. A magnetic field or a residual magnetic field is generated due to the electric signal, and becomes noise when detected by the magnetic sensor 122. Since the controller 104 is present at the location separated from the first opening 118b, the magnetic field or the residual magnetic field generated from the controller 104 hardly reaches the magnetic sensor 122. As a result, the magnetic sensor 122 can perform measurement with less noise.

The main body 118a is made of a ferromagnetic material having relative permeability of, for example, several thousands or more, or a conductor having high conductivity. As the ferromagnetic material, permalloy, ferrite, iron, chromium, cobalt-based amorphous metal, or the like may be used. As the conductor having high conductivity, for example, aluminum which has a magnetic field reduction function due to an eddy current effect may be used. The main body 118a may be formed by alternately stacking a ferromagnetic material and a conductor having high conductivity. In the present embodiment, for example, the main body 118a is formed by alternately staking an aluminum plate and a permalloy plate as two layers whose entire thickness is about 20 mm to 30 mm.

First correction coils (first Helmholtz coils 118c) as a magnetic shield unit are provided at ends on the +Y direction side and the −Y direction side of the main body 118a. The first Helmholtz coils 118c are coils for correcting an entering magnetic field which enters the internal space of the main body 118a. The entering magnetic field indicates an external magnetic field which passes through the first opening 118b and enters the internal space. The entering magnetic field is strongest with respect to the first opening 118b in the Y direction. The first Helmholtz coils 118c generate a magnetic field which cancels out the entering magnetic field by using a current supplied from the controller 104.

The table 121 includes a second foundation 123. The second foundation 123 is disposed on the bottom inside the main body 118a, and extends from the inside of the main body 118a to the outside of the first opening 118b through the first opening 118b in the Y direction (a movable direction of the subject 109). A pair of Y-direction rails 124 extending in the Y direction is provided on the second foundation 123. A Y-direction table 125 which is moved in the Y direction along the Y-direction rails 124 is provided on the Y-direction rails 124. A Y-direction linear motion mechanism 126 which moves the Y-direction table 125 is provided between the two Y-direction rails 124. The Y-direction linear motion mechanism 126 is coupled to the controller 104, and is operated in response to an instruction from the controller 104.

A Z-direction table 127 is provided on the Y-direction table 125, and a lifting device (not illustrated) is provided between the Y-direction table 125 and the Z-direction table 127. The lifting device lifts the Z-direction table 127. Four X-direction rails 128 extending in the X direction are provided on a surface on the +Z direction side of the Z-direction table 127. An X-direction table 129 which is moved in the X direction along the X-direction rails 128 is provided on the X-direction rails 128.

An X-direction linear movement mechanism 130 which moves the X-direction table 129 in the X direction is provided on the −Y direction side on the Z-direction table 127. The X-direction linear movement mechanism 130 includes a pair of bearing portions 130a, and the bearing portions 130a are provided to be erect on the Z-direction table 127. The X-direction table 129 is located between the two bearing portions 130a. The two bearing portions 130a rotatably support a first screw rod 130b. A first penetration hole (not illustrated) which penetrates in the X direction is provided in the X-direction table 129, and the first screw rod 130b is provided to penetrate through the first penetration hole of the X-direction table 129. A female screw (not illustrated) is formed on the first penetration hole, and the first screw rod 130b is engaged with the female screw. A first handle 130c is provided at one end on the −X direction side of the first screw rod 130b, and the first handle 130c is fixed to the first screw rod 130b. If the first handle 130c is rotated, the first screw rod 130b is rotated. Since the first screw rod 130b is engaged with the female screw of the X-direction table 129, if the first screw rod 130b is rotated, the X-direction table 129 is moved in the X direction. Therefore, an operator rotates the first handle 130c so as to move the X-direction table 129 in the X direction.

A second handle 131 is provided on a side surface of the X-direction table 129 directed in the −Y direction. The second handle 131 is joined to a second screw rod 131a. A second penetration hole (not illustrated) which intersects the first screw rod 130b is provided in the X-direction table 129, and the second screw rod 131a is inserted into the second penetration hole. A female screw is formed on the second penetration hole, and the second screw rod 131a is engaged with the female screw of the second penetration hole. When the operator rotates the second handle 131, the second screw rod 131a presses the first screw rod 130b so as to minimize of rotation of the first screw rod 130b. Therefore, it is possible to prevent the X-direction table 129 from being moved in the X direction by operating the second handle 131. The second foundation 123, the Y-direction rails 124, the Y-direction table 125, the Y-direction linear motion mechanism 126, the Z-direction table 127, the X-direction rails 128, the X-direction table 129, and the like constituting the table 121 are made of non-magnetic materials such as a wood, a resin, a ceramic, and non-magnetic metal.

In the electromagnetic shield device 118, a laser pointer 132 is provided on the +Z direction side of the first opening 118b. The laser pointer 132 emits laser light 132a in the −Z direction. The subject 109 is mounted to face upward on the table 121. The chest of the subject 109 is irradiated with the laser light 132a. The operator drives the Y-direction linear motion mechanism 126 to move the Y-direction table 125 in the Y direction. The operator operates the first handle 130c so as to move the X-direction table 129 in the X direction. Positions of the table 121 in the X direction and the Y direction may be adjusted so that xiphisternum 109e of the subject 109 is irradiated with the laser light 132a.

The magnetic sensor 122 is provided inside the magnetic field measurement section 103. The magnetic sensor 122 is a sensor which detects a magnetic field generated from the heart of the subject 109. The magnetic sensor 122 is fixed to the electromagnetic shield device 118. The location where the magnetic field measurement section 103 is disposed is adjusted to a state in which no magnetic field is substantially present by the electromagnetic shield device 118. Therefore, the magnetic sensor 122 can measure a magnetic field generated from the heart without being influenced by noise. The magnetic sensor 122 detects an intensity component of a magnetic field in a first direction 122a which is the same direction as the Z direction.

The controller 104 is provided with a display device 133 and an input device 134. The display device 133 is a liquid crystal display (LCD) or an organic light emitting diode (OLED). A measurement situation, a measurement result, and the like are displayed on the display device 133. The input device 134 is constituted of a keyboard, a rotary knob, or the like. An operator operates the input device 134 so as to input various instructions such as a measurement starting instruction or a measurement condition to the living body magnetic field measurement apparatus 101.

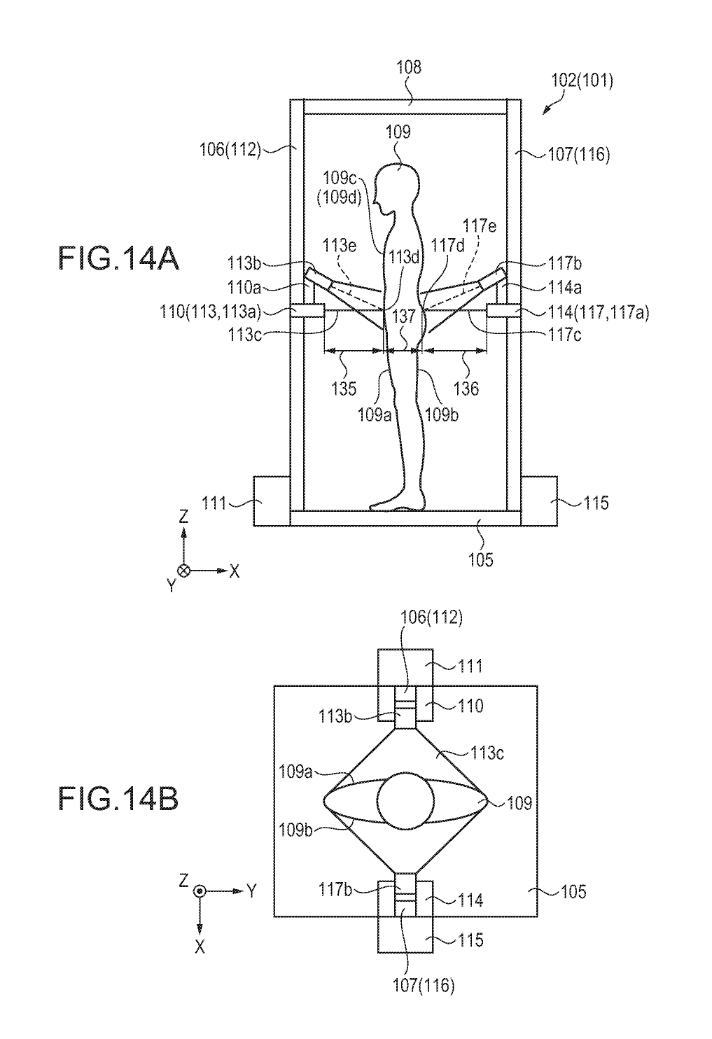

FIG. 14A is a schematic side view illustrating a structure of the contour measurement section, and FIG. 14B is a schematic top view illustrating a structure of the contour measurement section 102. In FIGS. 14A and 14B, the front sensor 113 includes a laser scanning unit 113a and an imaging device 113b. The laser scanning unit 113a is built into the front sensor 113, and emits laser light 113c in the X direction. The laser scanning unit 113a performs scanning with the laser light 113c in the Y direction. A front face 109a of the subject 109 is irradiated with the laser light 113c. The laser light 113c is reflected from the front face 109a. A reflection point 113d at which the laser light 113c is reflected from the front face 109a is linear when viewed from the front sensor 113.

The imaging device 113b is provided at the front stage 110 via a support portion 110a. The imaging device 113b is provided to be obliquely with respect to a traveling direction of the laser light 113c. The imaging device 113b images reflected light 113e which is reflected from the front face 109a of the subject 109. In this case, the laser scanning unit 113a, the reflection point 113d, and the imaging device 113b form a triangle. The distance between the laser scanning unit 113a and the imaging device 113b is a known value. An angle formed between the laser light 113c and the reflected light 113e can be detected on the basis of an image captured by the imaging device 113b. Therefore, the contour measurement section 102 can measure the distance between the front sensor 113 and the reflection point 113d by using a triangulation method. As a result, it is possible to measure a first distance 135 which is the distance between the front sensor 113 and the front face 109a of the subject 109.

The first motor 111 and the first linear movement mechanism 112 lift the front sensor 113. Consequently, the reflection point 113d is moved along the front face 109a of the subject 109. The first distance 135 at each location of the front face 109a of the subject 109 is measured. The first linear movement mechanism 112 is provided with a length measurement device (not illustrated). A position of the front stage 110 in the Z direction can be detected by using the length measurement device. The length measurement device includes a glass plate with a scale, and an optical sensor detecting the scale. The optical sensor detects a position of the glass plate. The laser light 113c is horizontally applied, and thus a position of the reflection point 113d in the Z direction can be detected. Since the laser light 113c is linearly applied to the front face 109a, the contour measurement section 102 can measure a stereoscopic shape of the front face 109a of the subject 109.

Similarly, the back sensor 117 includes a laser scanning unit 117a and an imaging device 117b. The laser scanning unit 117a is built into the back sensor 117, and emits laser light 117c in the −X direction. The laser scanning unit 117a performs scanning with the laser light 117c in the Y direction. The laser light 117c is applied to a back face 109b as a second face of the subject 109. The laser light 117c is reflected from the back face 109b. A reflection point 117d where the laser light 117c is reflected from the back face 109b is linear when viewed from the back sensor 117.

The imaging device 117b is provided at the back stage 114 via a support portion 114a. The imaging device 117b is provided to be obliquely with respect to a traveling direction of the laser light 117c. The imaging device 117b images reflected light 117e which is reflected from the back face 109b of the subject 109. In this case, the laser scanning unit 117a, the reflection point 117d, and the imaging device 117b form a triangle. The distance between the laser scanning unit 117a and the imaging device 117b is a known value. An angle formed between the laser light 117c and the reflected light 117e can be detected on the basis of an image captured by the imaging device 117b. Therefore, the contour measurement section 102 can measure the distance between the back sensor 117 and the reflection point 117d by using a triangulation method. As a result, it is possible to measure a second distance 136 which is the distance between the back sensor 117 and the back face 109b of the subject 109.

The second motor 115 and the second linear movement mechanism 116 lift the back sensor 117. Consequently, the reflection point 117d is moved along the back face 109b of the subject 109. The second distance 136 at each location of the back face 109b of the subject 109 is measured. The second linear movement mechanism 116 is provided with a length measurement device (not illustrated). A position of the back stage 114 in the Z direction can be detected by using the length measurement device. The laser light 117c is horizontally applied, and thus a position of the reflection point 117d in the Z direction can be detected. Therefore, the contour measurement section 102 can measure a shape of the back face 109b of the subject 109. Since a distance in the X direction between the front sensor 113 and the back sensor 117 is also a known value, it is possible to measure a subject width 137 which is the distance between the reflection point 113d and the reflection point 117d by subtracting the first distance 135 and the second distance 136 from the distance between the front sensor 113 and the back sensor 117.

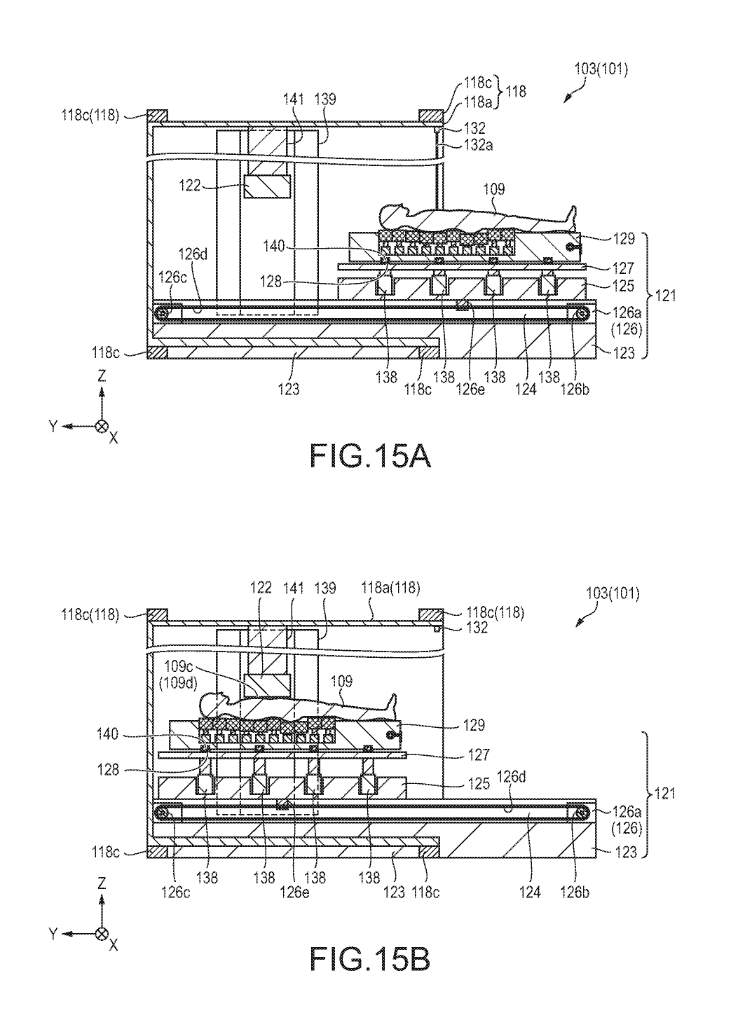

FIGS. 15A and 15B are schematic sectional views illustrating a structure of the table. FIG. 15A illustrates a state in which the table 121 is being moved in the −Y direction, and FIG. 15B illustrates a state in which the table 121 is moved into the magnetic field measurement section 103, and a heart magnetic field of the subject 109 is being measured. As illustrated in FIG. 15A, the pair of first Helmholtz coils 118c is disposed on the second foundation 123. A shape of the first Helmholtz coil 118c is a frame shape, and is disposed to surround the main body 118a.

The Y-direction linear motion mechanism 126 includes a motor 126a. A first pulley 126b is provided on a rotation shaft of the motor 126a, and a second pulley 126c is rotatably provided at an end on the Y direction side of the Y-direction linear motion mechanism 126. A timing belt 126d is hung on the first pulley 126b and the second pulley 126c. A connection portion 126e is provided on the timing belt 126d, and the connection portion 126e connects the timing belt 126d to the Y-direction table 125. When the motor 126a rotates the first pulley 126b, the connection portion 126e is moved in the Y direction by the torque of the motor 126a. The Y-direction table 125 is moved due to the movement of the connection portion 126e. Therefore, the motor 126a can move the Y-direction table 125 in the Y direction. The motor 126a changes a rotation direction of the first pulley 126b so as to move the Y-direction table 125 in both directions such as the +Y direction and the −Y direction.

Materials of the Y-direction rails 124, the second pulley 126c, the timing belt 126d, and the connection portion 126e are non-magnetic materials. The timing belt 126d is made of rubber and resin. The Y-direction rails 124, the second pulley 126c, and the connection portion 126e are made of ceramics.

Four lifting devices 138 are provided side by side in the Y direction in the Y-direction table 125. Each of the lifting devices 138 has a structure in which three air cylinders are arranged in the X direction. The lifting device 138 expands and contracts the air cylinders so as to lift the Z-direction table 127. Each air cylinder is provided with a length measurement device (not illustrated), and thus the lifting device 138 can detect a movement amount of the Z-direction table 127. The respective air cylinders move the Z-direction table 127 by the same distance, and thus the lifting devices 138 can move the Z-direction table 127 in parallel. Pneumatic equipment such as a compressor and an electromagnetic valve (not illustrated) is provided in the controller 104. The lifting devices 138 are controlled by the controller 104.

The X-direction table 129 is provided with wheels 140 which are in contact with the X-direction rails 128. The wheels 140 are rotated, and thus the X-direction table 129 can be easily moved in the X direction. The Z-direction table 127, the X-direction rails 128, and the wheels 140 are made of ceramics which are non-magnetic materials.

The magnetic sensor 122 is provided on the ceiling of the main body 118a via a support member 141. A position of the center of the magnetic sensor 122 in the Z direction is the central position between the ceiling of the main body 118a and the bottom of the main body 118a. A position of the center of the magnetic sensor 122 in the X direction is the central position between a wall on the +X direction side of the main body 118a and a wall on the −X direction side thereof. The distance between the center of the magnetic sensor 122 in the Y direction and an end on the −Y direction side of the main body 118a is twice longer than the distance between the center of the magnetic sensor 122 and the wall on the +Y direction side of the main body 118a. If the center of the magnetic sensor 122 is located at this location, it is possible for the magnetic sensor 122 to be hardly influenced by a magnetic field which enters the electromagnetic shield device 118 from the outside thereof.

Second correction coils (second Helmholtz coils 139) having a cube frame shape are provided inside the electromagnetic shield device 118. Specifically, at least three correction coils are provided so as to be orthogonal to each other in the X direction, the Y direction, and the Z direction. In the second Helmholtz coil 139 orthogonal to the X direction, a measurement space in which the subject 109 is disposed during measurement and the magnetic sensor 122 are interposed between a pair of coils from the X direction (left/right direction). The second Helmholtz coil 139 orthogonal to the X direction may generate a magnetic field in the X direction and may cancel out an external magnetic field in the X direction so that an X component of a magnetic field of the measurement space and the space in which the magnetic sensor 122 is disposed is reduced to the extent or less of not having an adverse effect on measurement. In the second Helmholtz coil 139 orthogonal to the Y direction, the measurement space and the magnetic sensor 122 are interposed between two pairs of coils (that is, four coils) from the Y direction (front/rear direction). The second Helmholtz coil 139 may orthogonal to the Y direction generate a magnetic field in the Y direction and may cancel out an external magnetic field in the Y direction so that a Y component of a magnetic field of the measurement space and the space in which the magnetic sensor 122 is disposed is reduced to the extent or less of not having an adverse effect on measurement. Since the main body 118a has a tubular shape extending in the front/rear direction, and an entering magnetic field is considerable in the Y direction, two pairs of second Helmholtz coils 139 are provided regarding the Y direction. In the second Helmholtz coil 139 orthogonal to the Z direction, the measurement space and the magnetic sensor 122 are interposed between a pair of coils from the Z direction (upper/lower direction). The second Helmholtz coil 139 orthogonal to the Z direction may generate a magnetic field in the Z direction and may cancel out an external magnetic field in the Z direction so that a Z component of a magnetic field of the measurement space and the space in which the magnetic sensor 122 is disposed is reduced to the extent or less of not having an adverse effect on measurement. The second Helmholtz coil 139 has a square frame shape when viewed from each orthogonal direction side, and is disposed so that the central position of the square frame overlaps the central position of the magnetic sensor 122. A length of the side of the square is not particularly limited, but, in the present embodiment, a length of one side is equal to or more than 75 cm and equal to or less than 85 cm. In FIGS. 15A and 15B, the shape of the second Helmholtz coil 139 is a rectangular shape for better viewing, but is actually a square shape.

Four second Helmholtz coils 139 having the square frame shape are disposed at the same interval in the Y direction. When viewed from the X direction, an outer circumference of the second Helmholtz coil 139 has a square frame shape, and there is a structure in which two coils are disposed inside the square frame shape. The second Helmholtz coil 139 is disposed so that the central position of the square frame shape overlaps the central position of the magnetic sensor 122.

A shape of the second Helmholtz coil 139 viewed from the Z direction is the same as the shape viewed from the X direction. The second Helmholtz coil 139 is disposed so that the central position of the square frame shape overlaps the central position of the magnetic sensor 122.

The second Helmholtz coil 139 has such a shape, and thus it is possible to further reduce a disturbance magnetic field in the magnetic sensor 122. Particularly, it is possible to reduce the influence of a magnetic flux which comes from the −Y direction side of the electromagnetic shield device 118.

When the table 121 is located on the −Y direction side of the electromagnetic shield device 118, a half or more of the table 121 protrudes out of the electromagnetic shield device 118. Consequently, the subject 109 is easily mounted on the table 121. When the subject 109 is mounted on the table 121, a height from the floor to the nose of the subject 109 is smaller than a height from the floor to the surface on the −Z direction side of the magnetic sensor 122. Therefore, when the Y-direction table 125 is moved in the Y direction, the subject 109 does not interfere with the magnetic sensor 122.

As illustrated in FIG. 15B, the Y-direction table 125 is moved in the +Y direction, and then the Z-direction table 127 is moved up. At this time, the chest 109c of the subject 109 is located at a location opposing the magnetic sensor 122, and comes close to the magnetic sensor 122. The surface of the chest 109c is a measured surface 109d as a first surface.

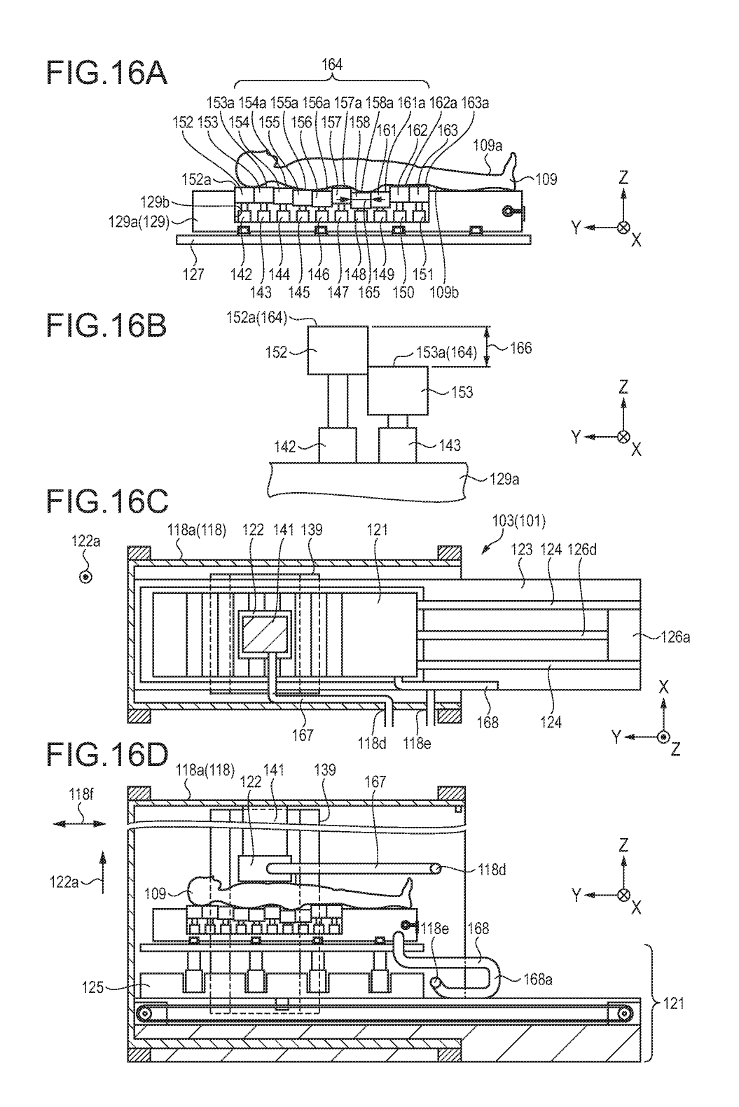

FIG. 16A is a side view illustrating a structure of the X-direction table. The X-direction table 129 includes a main body portion 129a, and the main body portion 129a is provided with a recess 129b on a surface on the Z direction side thereof. A length of the recess 129b in the X direction is the same as a width of the X-direction table 129 in the X direction. The recess 129b is located at a location opposing the head to the knee of the subject 109 mounted on the X-direction table 129. Ten lifting portions including a first lifting portion 142 to a tenth lifting portion 151 are provided side by side in the Y direction in the recess 129b. Each lifting portion has a structure in which three air cylinders are arranged in the X direction.

A first reception portion 152 to a tenth reception portion 163 are respectively provided on the +Z direction sides of the first lifting portion 142 to the tenth lifting portion 151. Each of the first reception portion 152 to the tenth reception portion 163 has a prism shape extending in the X direction. The first lifting portion 142 expands and contracts the three air cylinders so as to lift the first reception portion 152. Each air cylinder includes a length measurement device (not illustrated), and the first lifting portion 142 detects a movement amount of the first reception portion 152. The respective air cylinders move the first reception portion 152 by the same distance, and thus the first lifting portion 142 moves the first reception portion 152 in parallel.

The second lifting portion 143 to the tenth lifting portion 151 have the same structure as that of the first lifting portion 142. The second lifting portion 143 to the tenth lifting portion 151 can respectively move the second reception portion 153 to the tenth reception portion 163 in parallel. Pneumatic equipment such as a compressor and an electromagnetic valve (not illustrated) is provided in the controller 104. The controller 104 controls an amount of air supplied to the first lifting portion 142 to the tenth lifting portion 151, and thus controls each movement amount of the first lifting portion 142 to the tenth lifting portion 151.

Surfaces on the +Z direction sides of the first reception portion 152 to the tenth reception portion 163 are respectively a first division surface 152a to a tenth division surface 163a as division surfaces. The first division surface 152a to the tenth division surface 163a are collectively referred to as a contact surface 164. The contact surface 164 is a surface in contact with the subject 109 at the back face 109b. A shape of the contact surface 164 corresponds to a shape of the back face 109b.

The number of division surfaces constituting the contact surface 164 is preferably equal to or larger than 10 and equal to or smaller than 20, and more preferably fifteen. Since ten or more division surfaces are in contact with and support the subject 109, the subject 109 can be stably supported, and the measured surface 109d can be directed in the Z direction. The number of division surfaces is equal to or smaller than 20. Therefore, the controller 104 can easily control positions of the division surfaces. For better viewing, the number of division surfaces in FIG. 16A is 10.

A width 165 of each of the division surfaces constituting the contact surface 164 is equal to or more than 5 cm and equal to or less than 15 cm, and more preferably 10 cm. Since the division surfaces are in contact with and support the subject 109 at the interval of 5 cm to 15 cm, the subject 109 can be stably supported, and the measured surface 109d can be directed in the Z direction.

FIG. 16B is a main portion schematic enlarged view for explaining a movable range of the division surface. In FIG. 16B, the first lifting portion 142 is in a state of being expanded most. The second lifting portion 143 is in a state of being contracted most. In this case, a difference between the first division surface 152a and the second division surface 153a as a division surface is a movable range 166. The movable range 166 is preferably equal to or more than 3 cm and equal to or less than 10 cm. In this case, the contact surface 164 can match the shape of the back face 109b of the subject 109. Therefore, since the first division surface 152a to the tenth division surface 163a are in contact with and support the subject 109, the subject 109 can be stably supported, and the measured surface 109d can be directed in the +Z direction. Since the movable range is equal to or less than 10 cm, the controller 104 can easily control the division surfaces.

FIG. 16C is a top sectional view for explaining a configuration of a tube, and is a view in which the magnetic field measurement section 103 is cut on an XY plane crossing a support member 141. FIG. 16D is a side sectional view for explaining a configuration of the tube, and is a view in which the magnetic field measurement section 103 is cut on a YZ plane along the wall on the −Z direction side of the electromagnetic shield device 118.

The magnetic field measurement section 103 is provided with a first tube 167 as a tube and a second tube 168 as a tube. A wire through which electricity for driving the magnetic sensor 122 flows is provided in the first tube 167. The second tube 168 includes a tube through which air for driving the lifting devices 138 and the first lifting portion 142 to the tenth lifting portion 151 flows.

A second opening 118d and a third opening 118e are provided on a side surface of the −X direction side of the main body 118a. The first tube 167 is disposed through the second opening 118d and allows the inside and the outside of the electromagnetic shield device 118 to communicate with each other. In the second opening 118d, the first tube 167 extends in a direction orthogonal to the first direction 122a. In the second opening 118d, a direction of a magnetic vector passing through the first tube 167 is orthogonal to the first direction 122a. Therefore, the magnetic vector entering the electromagnetic shield device 118 through the first tube 167 hardly influences the magnetic sensor 122.

Similarly, the second tube 168 is disposed through the third opening 118e and causes the inside and the outside of the electromagnetic shield device 118 to communicate with each other. In the third opening 118e, the second tube 168 extends in a direction orthogonal to the first direction 122a. In the third opening 118e, a direction of a magnetic vector entering the electromagnetic shield device 118 through the second tube 168 is orthogonal to the first direction 122a. Therefore, the magnetic vector entering the electromagnetic shield device 118 through the second tube 168 hardly influences the magnetic sensor 122. As a result, the magnetic field measurement section 103 can perform measurement with less noise.

The direction in which the electromagnetic shield device 118 extends is set to a second direction 118f. The second direction 118f is a direction orthogonal to the first direction 122a. The first tube 167 extends in the second direction 118f along the main body 118a. Therefore, arrangement is obtained in which the first tube 167 is easily provided. The first tube 167 extends in the second direction 118f, and the second direction 118f is orthogonal to the first direction 122a. Therefore, a magnetic vector passing through the first tube 167 hardly influences the magnetic sensor 122. As a result, the magnetic field measurement section 103 can perform measurement with less noise.

The second tube 168 has a structure of tending to be bent, and the second tube 168 is bent twice at a bent portion 168a on the −Y direction side. When the Y-direction table 125 is moved in the Y direction, the bent portion 168a also is moved in the Y direction. Consequently, the second tube 168 can be moved with high durability without being twisted.

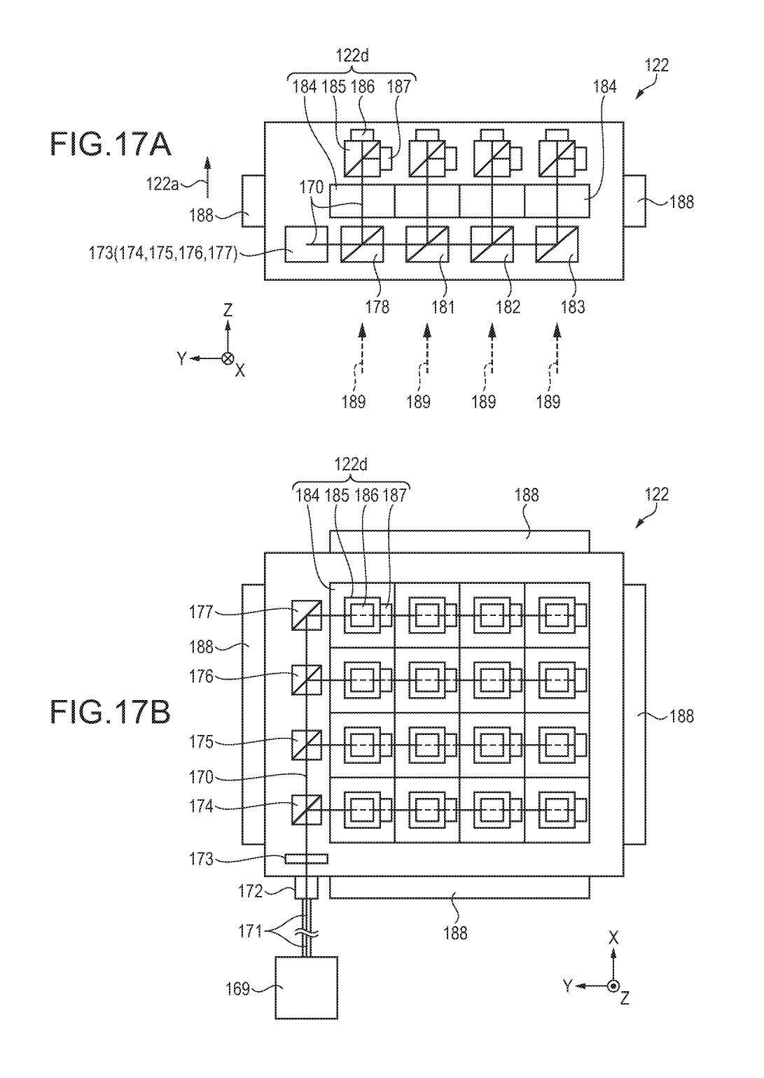

FIG. 17A is a schematic side view illustrating a structure of the magnetic sensor, and FIG. 17B is a schematic plan view illustrating a structure of the magnetic sensor. As illustrated in FIGS. 17A and 17B, laser light 170 is supplied from a laser light source 169 to the magnetic sensor 122. The laser light source 169 is provided in the controller 104, and the laser light 170 is supplied to the magnetic sensor 122 through an optical fiber 171 provided in the first tube 167. The magnetic sensor 122 is coupled to the optical fiber 171 via an optical connector 172.

The laser light source 169 outputs the laser light 170 with a wavelength corresponding to an absorption line of cesium. A wavelength of the laser light 170 is not particularly limited, but is set to a wavelength of 894 nm corresponding to the D1-line, in the present embodiment. The laser light source 169 is a tunable laser device, and the laser light 170 output from the laser light source 169 is continuous light with a predetermined light amount.

The laser light 170 supplied via the optical connector 172 travels in the +X direction and is applied to a polarization plate 173. The laser light 170 having passed through the polarization plate 173 is linearly polarized. The laser light 170 is sequentially applied to a first half mirror 174, a second half mirror 175, a third half mirror 176, and a first reflection mirror 177. Some of the laser light 170 is reflected by the first half mirror 174, the second half mirror 175, and the third half mirror 176 so as to travel in the −Y direction. The other light is transmitted therethrough so as to travel in the +X direction. The first reflection mirror 177 reflects the entire incident laser light 170 in the −Y direction. An optical path of the laser light 170 is divided into four optical paths by the first half mirror 174, the second half mirror 175, the third half mirror 176, and the first reflection mirror 177. Reflectance of each mirror is set so that light intensities of the laser light beams 170 on the respective optical paths are the same as each other.

Next, the laser light 170 is sequentially applied to a fourth half mirror 178, a fifth half mirror 181, a sixth half mirror 182, and a second reflection mirror 183. Some of the laser light 170 is reflected by the fourth half mirror 178, the fifth half mirror 181, and the sixth half mirror 182 so as to travel in the +Z direction. The other light 170 is transmitted therethrough so as to travel in the −Y direction. The second reflection mirror 183 reflects the entire incident laser light 170 in the +Z direction. A single optical path of the laser light 170 is divided into four optical paths by the fourth half mirror 178, the fifth half mirror 181, the sixth half mirror 182, and the second reflection mirror 183. Reflectance of each mirror is set so that light intensities of the laser light beams 170 on the respective optical paths are the same as each other. Therefore, the optical path of the laser light 170 is divided into the sixteen optical paths. In addition, reflectance of each mirror is set so that light intensities of the laser light beams 170 on the respective optical paths are the same as each other.

Gas cells 184 are provided on the respective optical paths of the laser light 170 on the +Z direction side of the fourth half mirror 178, the fifth half mirror 181, the sixth half mirror 182, and the second reflection mirror 183. The number of gas cells 184 is 16 in four rows and four columns. The laser light beams 170 reflected by the fourth half mirror 178, the fifth half mirror 181, the sixth half mirror 182, and the second reflection mirror 183 pass through the gas cells 184. The gas cell 184 is a box having a cavity therein, and an alkali metal gas is enclosed in the cavity. The alkali metal is not particularly limited, and potassium, rubidium, or cesium may be used. In the present embodiment, for example, cesium is used as the alkali metal.

A polarization separator 185 is provided on the +Z direction side of each gas cell 184. The polarization separator 185 is an element which separates the incident laser light 170 into two polarization components of the laser light 170, which are orthogonal to each other. As the polarization separator 185, for example, a Wollaston prism or a polarized beam splitter may be used.

A first photodetector 186 is provided on the +Z direction side of the polarization separator 185, and a second photodetector 187 is provided on the −Y direction side of the polarization separator 185. The laser light 170 having passed through the polarization separator 185 is applied to the first photodetector 186, and the laser light 170 reflected by the polarization separator 185 is applied to the second photodetector 187. The first photodetector 186 and the second photodetector 187 output currents corresponding to an amount of incident laser light 170 to the controller 104. If the first photodetector 186 and the second photodetector 187 generate magnetic fields, this may influence measurement, and thus it is preferable that the first photodetector 186 and the second photodetector 187 are made of a non-magnetic material. The magnetic sensor 122 includes heaters 188 which are provided on both sides in the X direction and both sides in the Y direction. Each of the heaters 188 preferably has a structure in which a magnetic field is not generated, and may employ, for example, a heater of a type of performing heating by causing steam or hot air to pass through a flow passage. Instead of using a heater, the gas cell 184 may be inductively heated by using a high frequency voltage.

The magnetic sensor 122 is disposed on the +Z direction side of the subject 109. A magnetic vector 189 generated from the subject 109 enters the magnetic sensor 122 from the −Z direction side. The magnetic vector 189 passes through the fourth half mirror 178 to the second reflection mirror 183, and then passes through the gas cell 184. The magnetic vector 189 passes through the polarization separator 185, and comes out of the magnetic sensor 122.

The magnetic sensor 122 is a sensor which is called an optical pumping type magnetic sensor or an optical pumping atom magnetic sensor. Cesium in the gas cell 184 is heated and is brought into a gaseous state. The cesium gas is irradiated with the linearly polarized laser light 170, and thus cesium atoms are excited so that orientations of magnetic moments can be aligned. When the magnetic vector 189 passes through the gas cell 184 in this state, the magnetic moments of the cesium atoms precess due to a magnetic field of the magnetic vector 189. This precession is referred to as Larmore precession. The magnitude of the Larmore precession has a positive correlation with the strength of the magnetic vector 189. In the Larmore precession, a polarization plane of the laser light 170 is rotated. The magnitude of the Larmore precession has a positive correlation with a change amount of a rotation angle of the polarization plane of the laser light 170. Therefore, the strength of the magnetic vector 189 has a positive correlation with the change amount of a rotation angel of the polarization plane of the laser light 170. The sensitivity of the magnetic sensor 122 is high in the first direction 122a in the magnetic vector 189, and is low in the direction orthogonal to the first direction 122a.

The polarization separator 185 separates the laser light 170 into two components of linearly polarized light which are orthogonal to each other. The first photodetector 186 and the second photodetector 187 respectively detect the strengths of the two components of linearly polarized light orthogonal to each other. Consequently, the first photodetector 186 and the second photodetector 187 can detect a rotation angle of a polarization plane of the laser light 170. The magnetic sensor 122 can detect the strength of the magnetic vector 189 on the basis of a change of the rotation angel of the polarization plane of the laser light 170. An element constituted of the gas cell 184, the polarization separator 185, the first photodetector 186, and the second photodetector 187 is referred to as a sensor element 122d. In the present embodiment, sixteen sensor elements 122d of four rows and four columns are disposed in the magnetic sensor 122. The number and arrangement of the sensor elements 122d in the magnetic sensor 122 are not particularly limited. The sensor elements 122d may be disposed in three or less rows or five or more rows. Similarly, the sensor elements 122d may be disposed in three or less columns or five or more columns. The larger the number of sensor elements 122d, the higher the spatial resolution.

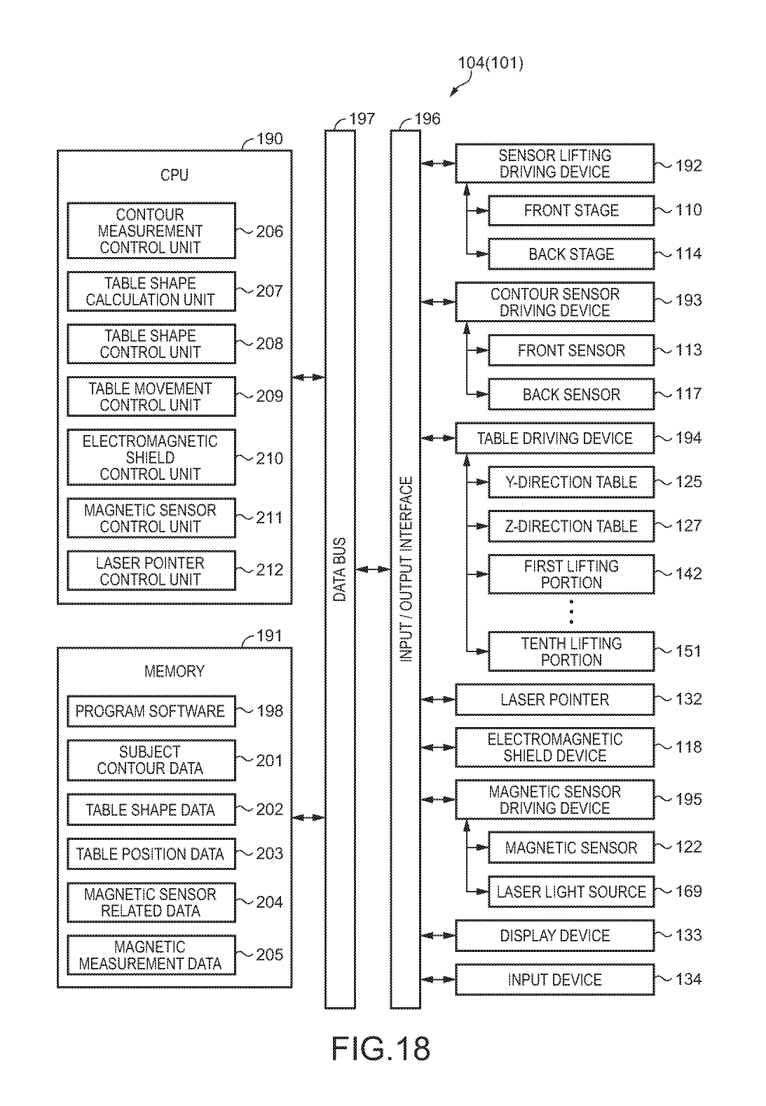

FIG. 18 is an electrical control block diagram of the controller. As illustrated in FIG. 18, the living body magnetic field measurement apparatus 101 includes the controller 104 controlling an operation of the living body magnetic field measurement apparatus 101. The controller 104 includes a central processing unit (CPU) 190 which performs various calculation processes as a processor, and a memory 191 which stores various information. A sensor lifting driving device 192, a contour sensor driving device 193, a table driving device 194, the laser pointer 132, the electromagnetic shield device 118, a magnetic sensor driving device 195, the display device 133, and the input device 134 are coupled to the CPU 190 via an input/output interface 196 and a data bus 197.

The sensor lifting driving device 192 is a device driving the front stage 110 and the back stage 114. The sensor lifting driving device 192 receives an instruction signal for moving positions of the front stage 110 and the back stage 114 from the CPU 190. The front stage 110 and the back stage 114 respectively include length measurement devices which detect positions thereof, and the sensor lifting driving device 192 detects positions of the front stage 110 and the back stage 114.

The sensor lifting driving device 192 calculates a difference between a position to which the front stage 110 is scheduled to be moved and the present position of the front stage 110. The sensor lifting driving device 192 drives the first motor 111 to move the front stage 110 to the position to which the first motor 111 is scheduled to be moved. Similarly, the sensor lifting driving device 192 calculates a difference between a position to which the back stage 114 is scheduled to be moved and the present position of the back stage 114. The sensor lifting driving device 192 drives the second motor 115 to move the back stage 114 to the position to which the back stage 114 is scheduled to be moved.

The contour sensor driving device 193 is a device driving the front sensor 113 and the back sensor 117. The contour sensor driving device 193 drives the laser scanning unit 113a to emit the laser light 113c toward the subject 109. The contour sensor driving device 193 causes scanning to be performed with the laser light 113c in the horizontal direction. The contour sensor driving device 193 drives the imaging device 113b to capture an image of the reflection point 113d. Similarly, the contour sensor driving device 193 drives the laser scanning unit 117a to emit the laser light 117c toward the subject 109. The contour sensor driving device 193 causes scanning to be performed with the laser light 117c in the horizontal direction. The contour sensor driving device 193 drives the imaging device 117b to capture an image of the reflection point 117d.

The table driving device 194 is a device driving the Y-direction table 125, the Z-direction table 127, and the first lifting portion 142 to the tenth lifting portion 151. The table driving device 194 receives an instruction signal for moving a position of the Y-direction table 125 from the CPU 190. The Y-direction table 125 includes a length measurement device which detects a position thereof, and the table driving device 194 detects a position of the Y-direction table 125. A difference between a position to which the Y-direction table 125 is scheduled to be moved and the present position of the Y-direction table 125 is calculated. The table driving device 194 drives the motor 126a to move the Y-direction table 125 to the position to which the Y-direction table 125 is scheduled to be moved. Consequently, the table driving device 194 can move the Y-direction table 125 between the position inside the electromagnetic shield device 118 and the position outside the electromagnetic shield device 118.

Similarly, the table driving device 194 receives an instruction signal for moving a position of the Z-direction table 127 from the CPU 190. Each of the lifting devices 138 lifting the Z-direction table 127 includes a length measurement device detecting position of the Z-direction table 127, and the table driving device 194 detects a position of the Z-direction table 127. A difference between a position to which the Z-direction table 127 is scheduled to be moved and the present position of the Z-direction table 127 is calculated. The lifting device 138 is an air cylinder, and the table driving device 194 is provided with pneumatic equipment such as a compressor or an electromagnetic valve driving the lifting device 138. The table driving device 194 controls an amount of air supplied to the lifting device 138 so as to move the Z-direction table 127 to the position to which the Z-direction table 127 is scheduled to be moved.

Similarly, the table driving device 194 receives an instruction signal for moving positions of the first division surface 152a to the tenth division surface 163a from the CPU 190. The first lifting portion 142 to the tenth lifting portion 151 which lift the first division surface 152a to the tenth division surface 163a respectively include length measurement devices measuring positions of the first division surface 152a to the tenth division surface 163a, and the table driving device 194 detects positions of the first division surface 152a to the tenth division surface 163a. A difference between a position to which each of the first division surface 152a to the tenth division surface 163a is scheduled to be moved and the present position of each surface is calculated. The first lifting portion 142 to the tenth lifting portion 151 are air cylinders, and the table driving device 194 is provided with pneumatic equipment such as a compressor or an electromagnetic valve driving the first lifting portion 142 to the tenth lifting portion 151. The table driving device 194 controls an amount of air supplied to the first lifting portion 142 to the tenth lifting portion 151 so as to move the first division surface 152a to the tenth division surface 163a to the positions to which the first division surface 152a to the tenth division surface 163a are scheduled to be moved.

The laser pointer 132 includes a light source emitting the laser light 132a. The laser pointer 132 receives an instruction from the CPU 190, and turns on and off the laser light 132a.

The electromagnetic shield device 118 includes the first Helmholtz coil 118c and a sensor detecting an internal magnetic field. The electromagnetic shield device 118 reduces an internal magnetic field of the main body 118a by driving the first Helmholtz coil 118c in response to an instruction from the CPU 190.

The magnetic sensor driving device 195 is a device driving the magnetic sensor 122 and the laser light source 169. The magnetic sensor 122 is provided with the first photodetector 186, the second photodetector 187, and the heater 188. The magnetic sensor driving device 195 drives the first photodetector 186, the second photodetector 187, and the heater 188. The magnetic sensor driving device 195 converts electric signals output from the first photodetector 186 and the second photodetector 187 into digital signals which are then output to the CPU 190. The magnetic sensor driving device 195 drives the heater 188 so that the magnetic sensor 122 is maintained at a predetermined temperature. The magnetic sensor driving device 195 drives the laser light source 169 to supply the laser light 170 to the magnetic sensor 122.

The display device 133 displays predetermined information in response to an instruction from the CPU 190. The operator operates the input device 134 on the basis of the display content and inputs the instruction content. The instruction content is transmitted to the CPU 190.

The memory 191 is a concept including a semiconductor memory such as a RAM or a ROM, a hard disk, and an external storage device such as a DVD-ROM. In terms of a function, a storage region for storing program software 198 in which control procedures of an operation of the living body magnetic field measurement apparatus 101 are described, or a storage region for storing subject contour data 201 which is data obtained by measuring a contour of the subject 109 is set. In addition, a storage region for storing table shape data 202 which is data indicating a shape of the contact surface 164 in the table 121 is set.

Further, a storing region for storing table position data 203 which is data indicating a position of the Y-direction table 125, the Z-direction table 127, and the X-direction table 129 is set. Still further, a storage region for storing magnetic sensor related data 204 which is data such as parameters used to drive the magnetic sensor 122 is set in the memory 191. Furthermore, a storage region for storing magnetic measurement data 205 which is data measured by the magnetic sensor 122 is set in the memory 191. Moreover, a storage region functioning as a work area for the CPU 190, a temporary file, or the like, and other various storage regions are set.

The CPU 190 controls measurement of a magnetic field generated from the heart of the subject 109 according to the program software 198 stored in the memory 191. As a specific function realizing unit, the CPU 190 includes a contour measurement control unit 206 which is a measurement unit. The contour measurement control unit 206 controls measurement of a contour of the subject 109 by lifting the front sensor 113 and the back sensor 117. The CPU 190 includes a table shape calculation unit 207 as a control unit. The table shape calculation unit 207 calculates a shape of the contact surface 164 in accordance with a shape of the subject 109. The CPU 190 includes a table shape control unit 208 as a control unit. The table shape control unit 208 performs control for forming a shape of the contact surface 164 of the X-direction table 129 so that the shape becomes the same as a shape of the contact surface 164 calculated by the table shape calculation unit 207.

The CPU 190 includes a table movement control unit 209. The table movement control unit 209 controls movement and stoppage positions of the Y-direction table 125, the Z-direction table 127, and the X-direction table 129. The CPU 190 includes an electromagnetic shield control unit 210. The electromagnetic shield control unit 210 performs control for minimizing a magnetic field around the magnetic sensor 122 by driving the electromagnetic shield device 118.

The CPU 190 includes a magnetic sensor control unit 211. The magnetic sensor control unit 211 performs control for causing the magnetic sensor driving device 195 to drive the magnetic sensor 122 to detect the strength of the magnetic vector 189. The CPU 190 includes a laser pointer control unit 212. The laser pointer control unit 212 performs control for driving the laser pointer 132 to turn on and off the laser light 132a.

In the present embodiment, the above-described respective functions of the living body magnetic field measurement apparatus 101 are realized in the program software by using the CPU 190, but, in a case where the above-described respective functions can be realized by a stand-alone electronic circuit (hardware) without using the CPU 190, such an electronic circuit may be used.

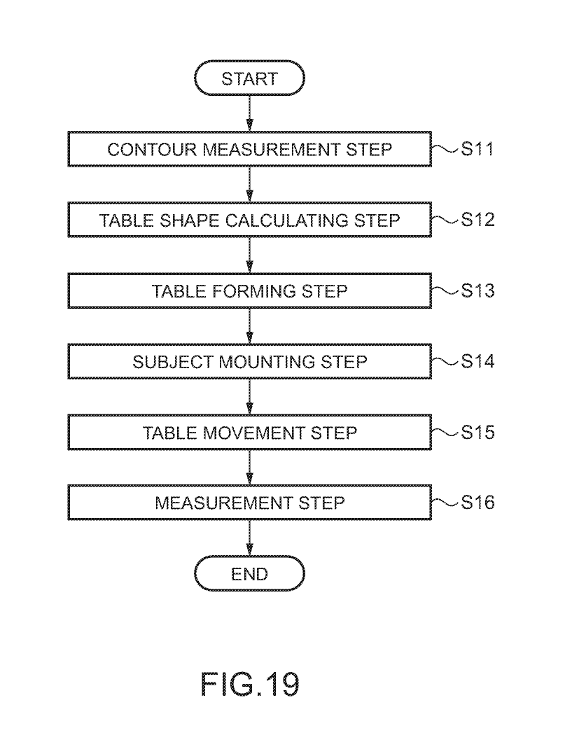

Next, a description will be made of a living body magnetic field measurement method using the above-described living body magnetic field measurement apparatus 101 with reference to FIGS. 14A and 14B and FIGS. 19 to 22B. FIG. 19 is a flowchart illustrating a living body magnetic field measurement method. In the flowchart illustrated in FIG. 19, step S11 corresponds to a contour measurement step. In this step, the contour measurement section 102 measures a contour of the subject 109. Next, the flow proceeds to step S12. Step S12 is a table shape calculation step. In this step, the table shape calculation unit 207 calculates a shape of the contact surface 164 of the X-direction table 129. Next, the flow proceeds to step S13.

Step S13 is a table forming step. In this step, the table shape control unit 208 forms a shape of the contact surface 164 of the X-direction table 129. Next, the flow proceeds to step S14. Step S14 is a subject mounting step. In this step, the subject 109 is mounted on the contact surface 164 of the X-direction table 129. Next, the flow proceeds to step S15. Step S15 is a table movement step. In this step, the table movement control unit 209 moves the table 121 so that the chest 109c of the subject 109 is moved to a location opposing the magnetic sensor 122. Next, the flow proceeds to step S16.

Step S16 is a measurement step. In this step, the magnetic sensor control unit 211 causes the magnetic sensor driving device 195 to drive the magnetic sensor 122 to measure a magnetic field coming out of the chest 109c of the subject 109. Through the above steps, the process of measuring a magnetic field of the subject 109 is finished.

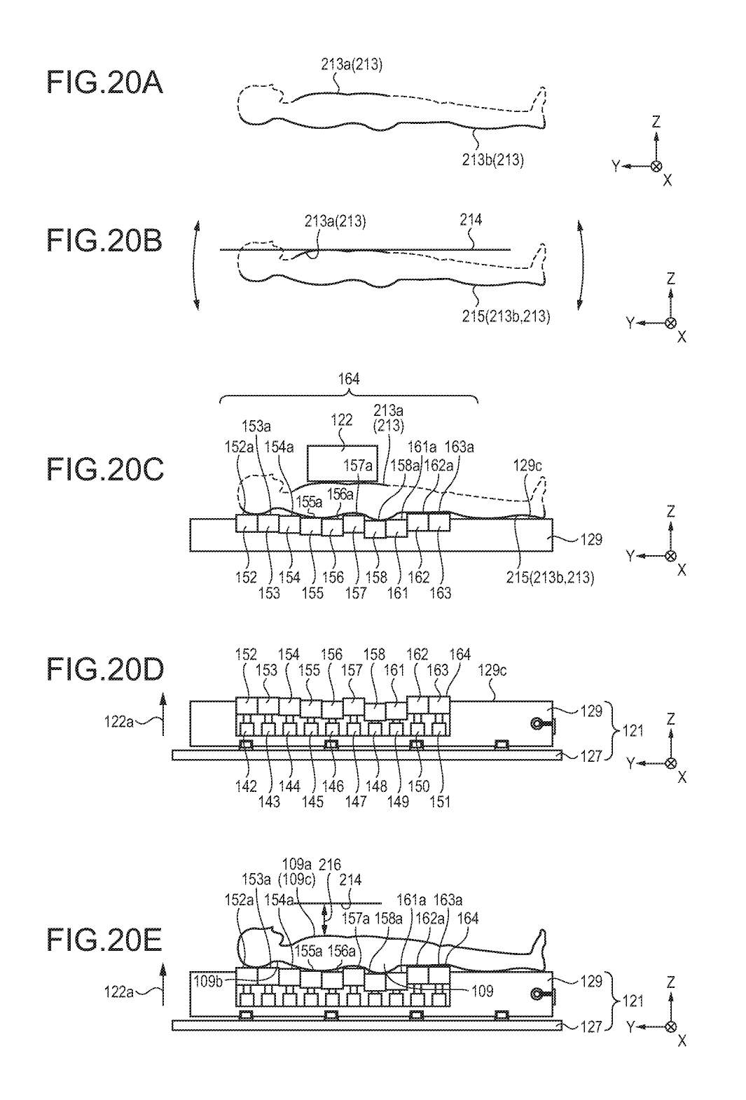

Next, with reference to FIGS. 14A and 14B, and FIG. 20A to FIG. 22B, the magnetic field measurement method will be described in more detail so as to correspond to the steps illustrated in FIG. 19. FIG. 20A to FIG. 22B are schematic diagrams for explaining the living body magnetic field measurement method.

FIGS. 14A and 14B, and FIG. 20A are diagrams corresponding to the contour measurement step of step S11. As illustrated in FIGS. 14A and 14B, the subject 109 is mounted on the first foundation 105. The subject 109 is in a standing attitude. The operator operates the input device 134 so as to input a distance from the first foundation 105 to the abdomen of the subject 109. The operator operates the input device 134 so as to input a distance from the first foundation 105 to the neck of the subject 109. The operator operates the input device 134 so as to input the height of the subject 109.

The contour measurement control unit 206 outputs an instruction signal to the sensor lifting driving device 192 so as to move the front stage 110 and the back stage 114. First, the contour measurement control unit 206 moves down the front stage 110 and the back stage 114 to the first foundation 105. The contour measurement control unit 206 outputs an instruction signal to the contour sensor driving device 193 so as to drive the back sensor 117. The back sensor 117 measures the second distance 136. The contour measurement control unit 206 performs both movement of the front stage 110 and the back stage 114 and measurement using the back sensor 117. Consequently, the contour measurement section 102 measures a shape of the back face 109b.

When the front sensor 113 is moved to the location opposing the abdomen of the subject 109, the contour measurement control unit 206 outputs an instruction signal to the contour sensor driving device 193 so as to drive the front sensor 113. The front sensor 113 measures the first distance 135. The contour measurement control unit 206 performs both movement of the front stage 110 and the back stage 114 and measurement using the front sensor 113 and the back sensor 117. Consequently, the contour measurement section 102 measures shapes of the front face 109a and the back face 109b.

When the front sensor 113 is moved to the location opposing the neck of the subject 109, the contour measurement control unit 206 outputs an instruction signal to the contour sensor driving device 193 so as to stop the driving of the front sensor 113. Thereafter, the contour measurement control unit 206 performs both movement of the front stage 110 and the back stage 114 and measurement using the back sensor 117. Consequently, the contour measurement section 102 measures a shape of the back face 109b.

When the front stage 110 and the back stage 114 arrive up to the height of the subject 109, the contour measurement control unit 206 outputs an instruction signal to the sensor lifting driving device 192 so as to stop the movement of the front stage 110 and the back stage 114. The contour measurement control unit 206 outputs an instruction signal to the contour sensor driving device 193 so as to stop the driving of the back sensor 117.

The contour sensor driving device 193 stores the measured data in the memory 191 as the subject contour data 201. As a result, as illustrated in FIG. 20A, a contour line 213 is formed. In FIG. 20A, the solid line portion is the contour line 213 indicated by the measured data. In FIG. 20A, the dotted line portion is a portion corresponding to no data. In the contour line 213, a front face line 213a is a line corresponding to the chest 109c of the subject 109. The front face line 213a is a line from the abdomen to the neck of the subject 109. The subject contour data 201 is three-dimensional shape data indicating the surface of the chest 109c. The front face line 213a is a line intersecting the YZ plane passing through the center of the heart in the three-dimensional shape data. In the contour line 213, a back face line 213b is a diagram corresponding to the back face 109b of the subject 109. The back face line 213b is a line from the heel to the head of the subject 109.

FIGS. 20B and 20C are diagrams corresponding to the table shape calculation step of step S12. As illustrated in FIG. 20B, in step S12, a reference plane 214 is set. The reference plane 214 is parallel to the surface on the −Z direction side of the magnetic sensor 122, and corresponds to a virtual plane which is moved from the surface on the −Z direction side of the magnetic sensor 122 by 5 mm in the −Z direction. A normal direction of the reference plane 214 is the first direction 122a. The table shape calculation unit 207 rotates the contour line 213 with the X direction as a rotation axis. The contour line 213 is moved in the +Z direction or the −Z direction so that the front face line 213a is in contact with the reference plane 214. The front face line 213a is a line corresponding to the measured surface 109d. Rotation and movement of the graphic are calculated by using affine transform. The back face line 213b when the front face line 213a is in contact with the reference plane 214 is set to a subject back face line 215. The subject back face line 215 is a line used as a reference of a shape of the contact surface 164. In other words, the table shape calculation unit 207 calculates a shape of the back face 109b when the normal direction of the measured surface 109d is set to the first direction 122a.

As illustrated in FIG. 20C, the contact surface 164 is calculated so as to be in contact with the subject back face line 215. First, the subject back face line 215 is disposed on the X-direction table 129 so that a portion of the subject back face line 215 corresponding to the calf of the leg is in contact with an upper surface 129c of the X-direction table 129. The upper surface 129c of the X-direction table 129 is a surface directed in the +Z direction side. Next, the table shape calculation unit 207 sets a position of the tenth reception portion 163 so that the tenth division surface 163a is in contact with the subject back face line 215.

Successively, the table shape calculation unit 207 sets a position of the ninth reception portion 162 so that the ninth division surface 162a as a division surface is in contact with the subject back face line 215. Next, the table shape calculation unit 207 sets respective positions of the eighth reception portion 161 to the first reception portion 152 so that the eighth division surface 161a to the first division surface 152a are in contact with the subject back face line 215. The set first division surface 152a to tenth division surface 163a correspond to the contact surface 164. In this case, the surface in contact with the front face line 213a is a parallel surface which is separated from the surface on the −Z direction side of the magnetic sensor 122 by 5 mm.

FIG. 20D is a diagram corresponding to the table forming step of step S13. As illustrated in FIG. 20D, in step S13, the table shape control unit 208 adjusts the heights of the first lifting portion 142 to the tenth lifting portion 151. Positions of the first reception portion 152 to the tenth reception portion 163 in the Z direction are set to the positions of the first reception portion 152 to the tenth reception portion 163 set in step S12. As a result, the contact surface 164 on the X-direction table 129 is in contact with the subject back face line 215 at a plurality of locations. In other words, in steps S11 to step S13, when the normal direction of the measured surface 109d is the same as the first direction 122a, the contour measurement control unit 206, the table shape calculation unit 207, the table shape control unit 208 control a shape of the contact surface 164 to be a shape corresponding to the shape of the back face 109b.

FIG. 20E is a diagram corresponding to the subject mounting step of step S14. As illustrated in FIG. 20E, in step S14, the subject 109 is mounted on the contact surface 164 of the table 121. Since the contact surface 164 has the shape corresponding to the shape of the back face 109b of the subject 109, the back face 109b of the subject 109 is in contact with the contact surface 164. In this case, the back face 109b of the subject 109 is in contact with each of the first division surface 152a to the tenth division surface 163a, and thus the subject 109 is stably mounted on the table 121. The surface on the −Z direction side of the magnetic sensor 122, the reference plane 214, and the surface of the chest 109c are parallel to each other. The surface on the −Z direction side of the magnetic sensor 122 is separated from the reference plane 214 by 5 mm. In this step, the Z-direction table 127 is located at a low position, and thus the reference plane 214 is separated from the surface of the chest 109c by a predetermined reference height 216.

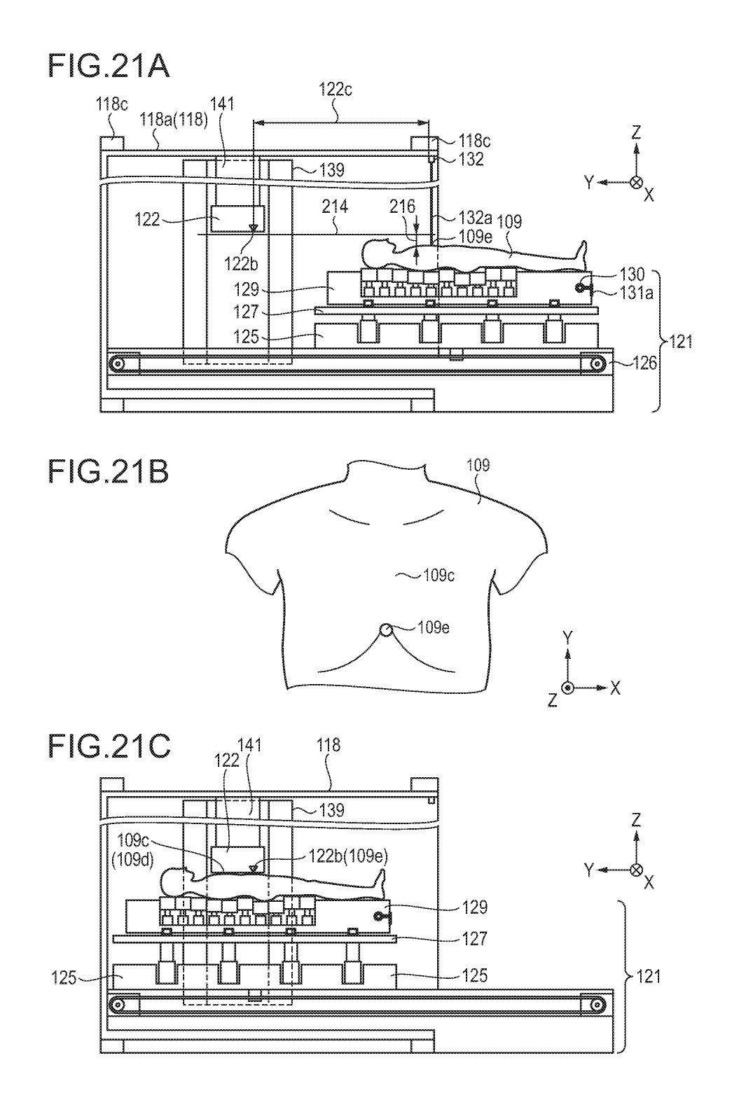

FIGS. 21A to 21C are diagrams corresponding to the table movement step of step S15. As illustrated in FIG. 21A, in step S15, the laser pointer 132 performs irradiation with the laser light 132a in the −Z direction. The operator operates the first handle 130c of the X-direction linear movement mechanism 130 to move the X-direction table 129. The operator operates the input device 134 of the controller 104 so that the Y-direction linear motion mechanism 126 is driven. The Y-direction linear motion mechanism 126 moves the Y-direction table 125 in the Y direction.

As illustrated in FIG. 21B, the xiphisternum 109e is present on the −Y direction side of the chest 109c in the subject 109. The xiphisternum 109e is a protrusion which protrudes at the lower end of sternum, and is present at a part called the pit of the stomach at which the rib bows join together. Referring to FIG. 21A again, the operator moves the Y-direction table 125 and the X-direction table 129 so as to move a position of the subject 109. The xiphisternum 109e is irradiation with the laser light 132a. Thereafter, the operator operates the input device 134 so as to input information indicating that positioning of the subject 109 has been finished.

A reference point 122b for checking a measurement point is set in the magnetic sensor 122. A position of the reference point 122b in the X direction is the same as the position in the X direction through the laser light 132a passes. A distance in the Y direction between the position of the reference point 122b and the position through which the laser light 132a passes is set to a predetermined reference distance 122c.

As illustrated in FIG. 21C, successively, the table movement control unit 209 outputs an instruction signal for moving the Y-direction table 125, to the table driving device 194. The table driving device 194 receives the instruction signal, and moves the Y-direction table 125 in the +Y direction by the reference distance 122c. Next, the table movement control unit 209 outputs an instruction signal for moving the Z-direction table 127, to the table driving device 194. The table driving device 194 receives the instruction signal, and moves up the Z-direction table 127 in the +Z direction by the reference height 216. Consequently, the measured surface 109d matches the reference plane 214.

As a result, the reference point 122b is located at a location opposing the xiphisternum 109e, and the measured surface 109d is located at a location opposing the magnetic sensor 122. The distance between the surface on the −Z direction side of the magnetic sensor 122 and the measured surface 109d is 5 mm. The operator checks whether or not the surface on the −Z direction side of the magnetic sensor 122 comes into contact with the measured surface 109d when the subject 109 takes a deep breath. In a case where the magnetic sensor 122 comes into contact with the subject 109, the operator moves down the Z-direction table 127. The operator operates the input device 134 so as to give an instruction to the table movement control unit 209. Consequently, the magnetic sensor 122 is not in contact with the subject 109 even when the subject 109 takes a deep breath.

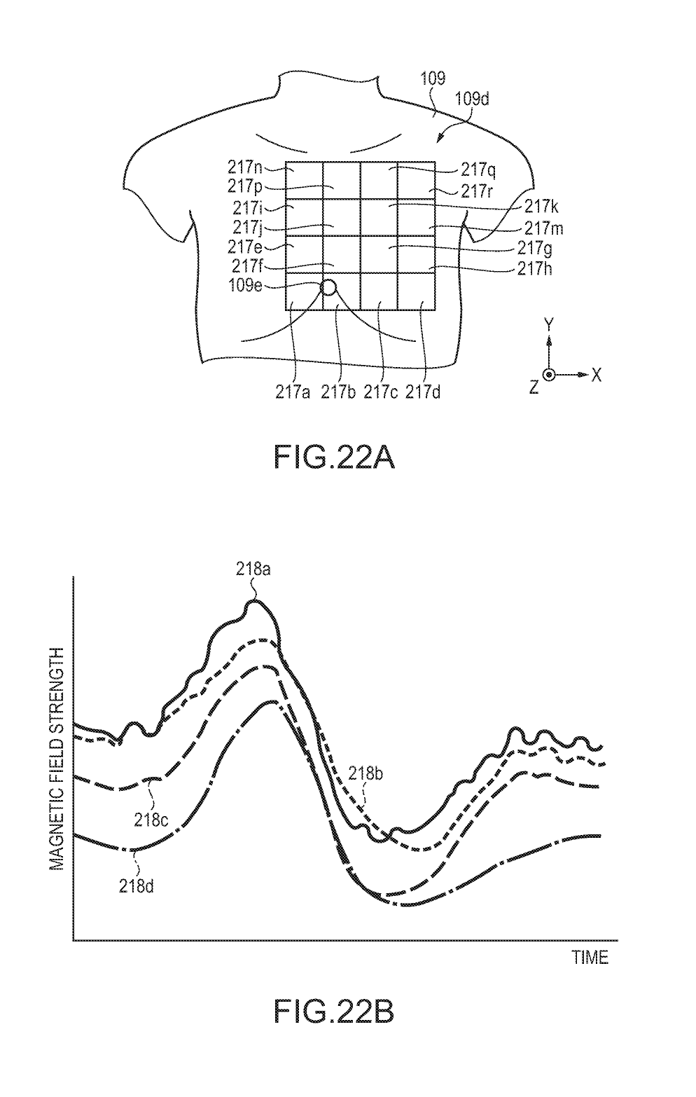

FIGS. 22A and 22B are diagrams corresponding to the measurement step of step S16. As illustrated in FIG. 22A, in step S16, the magnetic sensor 122 detects the magnetic vector 189 which travels in the Z direction from the measured surface 109d of the subject 109. The magnetic sensor control unit 211 outputs an instruction signal for starting measurement to the magnetic sensor driving device 195. The magnetic sensor driving device 195 receives the instruction signal for starting measurement, and causes the laser light source 169 to apply the laser light 170. If light emission of the laser light source 169 is stabilized, and the magnetic sensor 122 is stabilized at a predetermined temperature, the measurement is started. The strength of a magnetic field detected by the magnetic sensor 122 is output as an electric signal. The magnetic sensor driving device 195 converts the electric signal indicating the strength of the magnetic field into digital data which is then transmitted to the memory 191 as the magnetic measurement data 205.

In FIG. 22A, a first region 217a to a sixteenth region 217r indicate regions where the respective sensor elements 122d detect the magnetic vector 189. The first region 217a to the sixteenth region 217r are disposed in a lattice form of four rows and four columns. The xiphisternum 109e is disposed in the second region 217b. In this arrangement, the magnetic sensor 122 can detect the magnetic vector 189 generated from the heart of the subject 109 within the range of the first region 217a to the sixteenth region 217r.

FIG. 22B illustrates an example of change data of a magnetic field detected by the magnetic sensor 122. A longitudinal axis expresses the magnetic field strength, and the strength of an upper part in FIG. 22B is higher than the strength of a lower part therein. A transverse axis expresses a change in time, and time changes from a left part to a right part in FIG. 22B. The strength of the magnetic vector 189 detected by the sensor element 122d is referred to as the magnetic field strength. A first change line 218a indicates a change in the magnetic field strength in the twelfth region 217m, and indicates a change in the magnetic field strength on the upper left side of the heart. The upper left side of the heart represents a position in the X direction and the Y direction. A second change line 218b indicates a change in the magnetic field strength in the fourth region 217d, and indicates a change in the magnetic field strength on the lower left side of the heart. A third change line 218c indicates a change in the magnetic field strength in the second region 217b, and indicates a change in the magnetic field strength on the lower right side of the heart. A fourth change line 218d indicates a change in the magnetic field strength in the tenth region 217j, and indicates a change in the magnetic field strength on the upper right side of the heart. Sixteen magnetic field strength change lines can be obtained from the magnetic sensor 122. In FIG. 22B, for better viewing, four change lines are illustrated.

The first change line 218a has a peak, and then the second change line 218b has a peak. Next, the third change line 218c has a peak, and then the fourth change line 218d has a peak. As mentioned above, the peaks of the magnetic field strength move around the heart. When the heart does not normally operate, waveforms of the first change line 218a to the fourth change line 218d are deformed. Therefore, the operator can diagnose heart diseases of the subject 109 by observing waveforms of the first change line 218a to the fourth change line 218d.

After the measurement of a magnetic field is completed, the Z-direction table 127 is moved down, and the Y-direction table 125 is moved in the −Y direction. The subject 109 is demounted from the table 121, and thus the process of measuring a magnetic field from the heart of the subject 109 is finished.

As described above, according to the present embodiment, the following effects are achieved.

(1) According to the present embodiment, the magnetic sensor 122 detects a distribution of a component in the first direction 122a of the magnetic vector 189 of the measured surface 109d of the subject 109. The magnetic sensor 122 has high sensitivity for a component of the magnetic vector 189 in the first direction 122a, and has low sensitivity for a component thereof orthogonal to the first direction 122a. The measured surface 109d of the subject 109 is directed toward the magnetic sensor 122. The back face 109b is directed toward the table 121, and the back face 109b is in contact with the contact surface 164 of the table 121. The contour measurement section 102 measures shapes of the measured surface 109d and the back face 109b. The contact surface 164 of the table can be controlled. The table shape calculation unit 207 and the table shape control unit 208 control the contact surface 164 to have a shape corresponding to the back face 109b, and set the normal direction of the measured surface 109d of the subject 109 to be the same as the first direction 122a.

Therefore, the normal direction of the measured surface 109d of the subject 109 can be adjusted to a direction in which the sensitivity of the magnetic sensor 122 is high. If the normal direction of the measured surface 109d is inclined with respect to the first direction 122a, there may be the occurrence of a location where the distance between the measured surface 109d and the magnetic sensor 122 is short and a location where the distance therebetween is long. The weaker strength of the magnetic vector 189 is detected at the location where the distance between the measured surface 109d and the magnetic sensor 122 is short than at the location where the distance therebetween is long, and thus detection accuracy is reduced. In the present embodiment, the normal direction of the measured surface 109d is the same as the first direction 122a in which the magnetic vector 189 is detected. As a result, it is possible to detect a distribution of the magnetic vector 189 of the subject 109 with high accuracy.

(2) According to the present embodiment, the contact surface 164 is divided into a plurality of division surfaces moved in the first direction 122a. Positions of the plurality of division surfaces in the first direction 122a match the subject 109, and thus the contact surface 164 can be made to correspond to a shape of the back face 109b. The number of the plurality of division surfaces is equal to or larger than 10. Therefore, since the ten or more division surfaces are in contact with and support the subject 109, the subject 109 can be stably supported, and the measured surface 109d can be made to be directed in a predetermined direction. The number of the plurality of division surfaces is equal to or smaller than 20. Therefore, the table shape control unit 208 can easily control positions of the division surfaces.

(3) According to the present embodiment, a width of each of the first division surface 152a to the tenth division surface 163a is equal to or more than 5 cm and equal to or less than 15 cm, and more preferably 10 cm. Therefore, since the division surfaces are in contact with and support the subject 109 at the interval of 5 cm to 15 cm, the subject 109 can be stably supported, and the measured surface 109d can be directed in the first direction 122a.

(4) According to the present embodiment, a movable range of the first division surface 152a to the tenth division surface 163a in the first direction 122a is equal to or more than 3 cm and equal to or less than 10 cm. Therefore, the contact surface 164 can match the shape of the back face 109b of the subject 109. Thus, since the division surfaces are in contact with and support the subject 109, the subject 109 can be stably supported, and the measured surface 109d can be directed in the first direction 122a. Since the movable range is equal to or less than 10 cm, it is possible to easily control the division surfaces.

(5) According to the present embodiment, the electromagnetic shield device 118 attenuates an entering magnetic field line. The magnetic sensor 122 and the table 121 are provided inside the electromagnetic shield device 118. The electromagnetic shield device 118 is provided with the first opening 118b, and the subject 109 can come in and out of the electromagnetic shield device 118 via the first opening 118b. The controller 104 is located at a location separated from the first opening 118b.

The controller 104 makes an electric signal flow so as to control the table 121. A magnetic field is generated due to the electric signal, and becomes noise when detected by the magnetic sensor 122. In the present embodiment, since the controller 104 is present at the location separated from the first opening 118b, the magnetic field generated from the controller 104 hardly reaches the magnetic sensor 122. As a result, the magnetic sensor 122 can perform measurement with less noise.

(6) According to the present embodiment, the electromagnetic shield device 118 is provided with the first tube 167 and the second tube 168, and the first tube 167 and the second tube 168 extend in a direction orthogonal to the first direction 122a and allow the inside and the outside of the electromagnetic shield device 118 to communicate with each other. Directions of magnetic vectors passing through the first tube 167 and the second tube 168 are orthogonal to the first direction 122a. Therefore, magnetic vectors passing through the first tube 167 and the second tube 168 hardly influence the magnetic sensor 122. As a result, the magnetic sensor 122 can perform measurement with less noise.

(7) According to the present embodiment, the first tube 167 and the second tube 168 extend in the second direction 118f, and the second direction 118f is orthogonal to the first direction 122a. Therefore, magnetic vectors passing through the first tube 167 and the second tube 168 hardly influence the magnetic sensor 122. As a result, the magnetic sensor 122 can perform measurement with less noise. The first tube 167 and the second tube 168 are provided along the electromagnetic shield device 118, and arrangement is obtained in which the first tube 167 the second tube 168 are easily provided.

(8) According to the present embodiment, in the contour measurement step of step S11, shapes of the measured surface 109d and the back face 109b of the subject 109 are measured. In the table shape calculation step of step S12, a shape of the back face 109b is calculated when a normal direction of the measured surface 109d is set to be the same as a direction of the first direction component. In the table forming step of step S13, the contact surface 164 of the table 121 is formed to correspond to the calculated shape of the back face 109b. In the subject mounting step of step S14, the subject 109 is mounted on the contact surface 164 of the table 121. The contact surface 164 has a shape corresponding to the back face 109b of the subject 109, and the subject 109 is mounted so that the back face 109b thereof is in contact with the contact surface 164. Therefore, the measured surface 109d of the subject 109 can be directed in the first direction 122a.

### Third Embodiment

In the present embodiment, a description will be made of characteristic examples of a living body magnetic field measurement method of measuring a heart magnetic field generated from the heart by using a magnetic field measurement apparatus. A difference between the present embodiment and the second embodiment is that the table 121 is moved into the electromagnetic shield device 118, and then the contact surface 164 is changed. A description of the same content as that in the second embodiment will not be repeated.

In the present embodiment, the same steps as the steps in the second embodiment are performed. The same steps as the contour measurement step of step S11 to the subject mounting step of step S14 in the second embodiment are performed. In the table movement step of step S15, first, the table movement control unit 209 moves the Y-direction table 125. The Y-direction table 125 moves the measured surface 109d to a location opposing the magnetic sensor 122.

Next, the table shape control unit 208 controls the first lifting portion 142 to the tenth lifting portion 151 to deform the contact surface 164 to a shape corresponding to the shape of the back face line 213b. Successively, the table movement control unit 209 controls the lifting devices 138 so that the measured surface 109d comes close to the magnetic sensor 122. The table driving device 194 receives an instruction signal so as to move up the Z-direction table 127 by the reference height 216 in the +Z direction. Consequently, the measured surface 109d matches the reference plane 214.

In the measurement step of step S16, the same step as the step in the second embodiment is performed. Also in the above-described method, the normal direction of the measured surface 109d is the same as the first direction 122a in which the magnetic vector 189 is detected. As a result, it is possible to detect a distribution of the magnetic vector 189 of the subject 109 with high accuracy.

### Fourth Embodiment

In the present embodiment, with reference to the drawings, a description will be made of characteristic examples of a living body magnetic field measurement apparatus and a living body magnetic field measurement method of measuring a heart magnetic field generated from the heart by using the magnetic field measurement apparatus. A difference between the present embodiment and the first embodiment is that a most protruding portion of a measured surface is detected and is made to come close to a magnetic sensor. A description of the same content as that in the first embodiment will not be repeated.

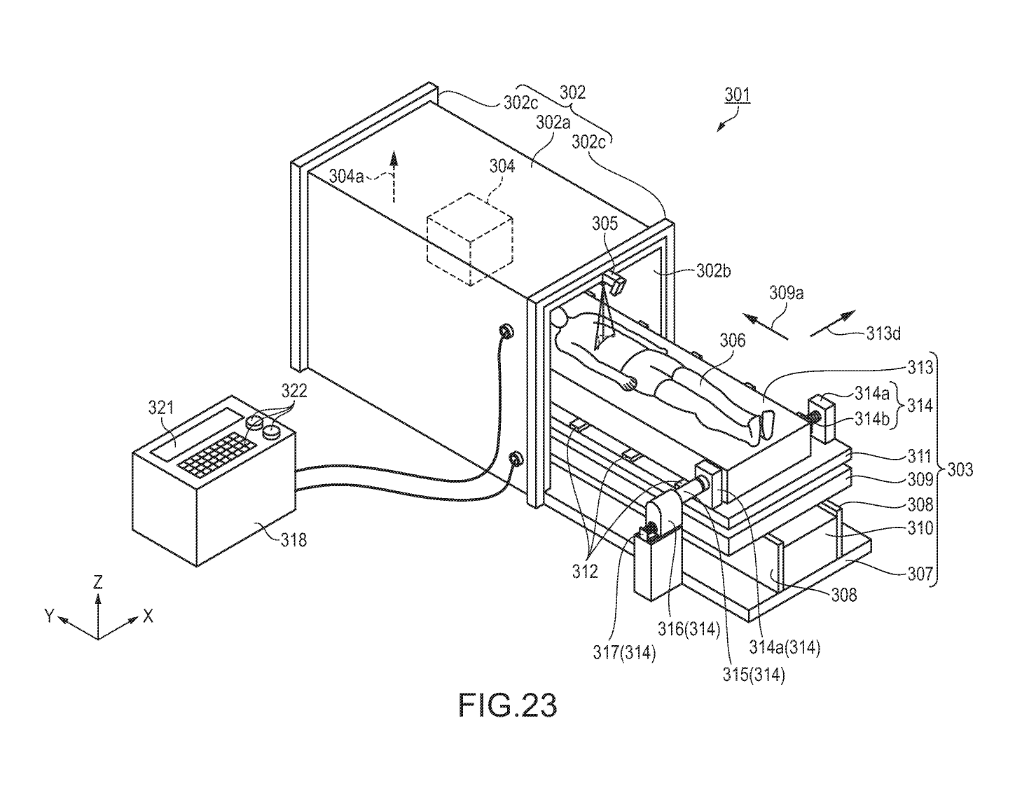

In the present embodiment, with reference to the drawings, a description will be made of characteristic examples of a living body magnetic field measurement apparatus and a living body magnetic field measurement method of measuring a heart magnetic field generated from the heart by using the magnetic field measurement apparatus. With reference to FIGS. 23 to 30, a description will be made of a structure of a living body magnetic field measurement apparatus according to the present embodiment. FIG. 23 is a schematic perspective view illustrating a configuration of the magnetic field measurement apparatus. As illustrated in FIG. 23, a living body magnetic field measurement apparatus 301 mainly includes an electromagnetic shield device 302 as a magnetic shield unit, a table 303, a magnetic sensor 304 as a magnetic detection unit, and a position measurement device 305 as a position measurement unit and a guide light irradiation unit.

The electromagnetic shield device 302 includes a rectangular tubular main body 302a. A longitudinal direction of the main body 302a is set to a Y direction. The gravity direction is set to a −Z direction, and a direction orthogonal to the Y direction and the Z direction is set to an X direction. The electromagnetic shield device 302 prevents an external magnetic field such as terrestrial magnetism from entering a space where the magnetic sensor 304 is disposed. That is, the influence of the external magnetic field on the magnetic sensor 304 is minimized by the electromagnetic shield device 302, and a magnetic field in the location where the magnetic sensor 304 is present is considerably lower than the external magnetic field. The main body 302a extends in the Y direction, and the main body 302a functions as a passive magnetic shield. The inside of the main body 302a is hollow, and a sectional shape of surfaces (orthogonal planes in the Y direction in the XZ section) passing through the X direction and the Z direction is a substantially quadrangle shape. In the present embodiment a sectional shape of the main body 302a is a square shape. The electromagnetic shield device 302 is provided with a first opening 302b on the −Y direction side, and the table 303 protrudes out of the first opening 302b. The size of the electromagnetic shield device 302 is not particularly limited, but, in the present embodiment, for example, a length thereof in the Y direction is about 200 cm, and one side of the first opening 302b is about 90 cm. A subject 306 mounted on the table 303 can come in and out of the electromagnetic shield device 302 via the first opening 302b along with the table 303.

The main body 302a is made of a ferromagnetic material having relative permeability of, for example, several thousands or more, or a conductor having high conductivity. As the ferromagnetic material, permalloy, ferrite, iron, chromium, cobalt-based amorphous metal, or the like may be used. As the conductor having high conductivity, for example, aluminum which has a magnetic field reduction function due to an eddy current effect may be used. The main body 302a may be formed by alternately stacking a ferromagnetic material and a conductor having high conductivity. In the present embodiment, for example, the main body 302a is formed by alternately staking an aluminum plate and a permalloy plate as two layers whose entire thickness is about 20 mm to 30 mm.

First correction coils (first Helmholtz coils 302c) as a magnetic shield unit are provided at ends on the +Y direction side and the −Y direction side of the main body 302a. The first Helmholtz coils 302c are coils for correcting an entering magnetic field which enters the internal space of the main body 302a. The entering magnetic field indicates an external magnetic field which passes through the first opening 302b and enters the internal space. The entering magnetic field is strongest with respect to the first opening 302b in the Y direction. The first Helmholtz coils 302c generate a magnetic field which cancels out the entering magnetic field by using a current.

The table 303 is provided with a foundation 307. The foundation 307 is disposed on the bottom inside the main body 302a, and extends from the inside of the main body 302a to the outside of the first opening 302b through the first opening 302b in the Y direction (in which the subject 306 is movable). A pair of Y-direction rails 308 extending in the Y direction is provided on the foundation 307. A Y-direction table 309 which is moved in the Y direction as a second direction 309a along the Y-direction rails 308 is provided on the Y-direction rails 308. A Y-direction linear motion mechanism 310 which moves the Y-direction table 309 is provided between the two Y-direction rails 308.

A Z-direction table 311 is provided on the Y-direction table 309, and a lifting device (not illustrated) is provided between the Y-direction table 309 and the Z-direction table 311. The lifting device lifts the Z-direction table 311. Six X-direction rails 312 extending in the X direction are provided on a surface on the +Z direction side of the Z-direction table 311. An X-direction table 313 which is moved in the X direction along the X-direction rails 312 is provided on the X-direction rails 312.

An X-direction linear movement mechanism 314 which moves the X-direction table 313 in the X direction as a third direction 313d is provided on the −Y direction side on the Z-direction table 311. The X-direction linear movement mechanism 314 includes a pair of bearing portions 314a, and the bearing portions 314a are provided to be erect on the Z-direction table 311. The X-direction table 313 is located between the two bearing portions 314a. The two bearing portions 314a rotatably support a first screw rod 314b. A first penetration hole (not illustrated) which penetrates in the X direction is provided in the X-direction table 313, and the first screw rod 314b is provided to penetrate through the first penetration hole of the X-direction table 313. A female screw (not illustrated) is formed on the first penetration hole, and the first screw rod 314b is engaged with the female screw.

An attachment/detachment portion 315 is provided at one end on the −X direction side of the first screw rod 314b, and the attachment/detachment portion 315 is fixed to the first screw rod 314b. If the attachment/detachment portion 315 is rotated, the first screw rod 314b is rotated. Since the first screw rod 314b is engaged with the female screw of the X-direction table 313, if the first screw rod 314b is rotated, the X-direction table 313 is moved in the X direction. The attachment/detachment portion 315 is coupled to a rotation shaft of an X-direction table motor 316 as a driving source. The X-direction table motor 316 rotates the attachment/detachment portion 315 so as to move the X-direction table 313 in the X direction. The X-direction table motor 316 is coupled to a motor movement portion 317 which moves the X-direction table motor 316 in the X direction. The X-direction linear movement mechanism 314 is constituted of the bearing portions 314a, the first screw rod 314b, the attachment/detachment portion 315, the X-direction table motor 316, the motor movement portion 317, and the like. The foundation 307, the Y-direction rails 308, the Y-direction table 309, the Y-direction linear motion mechanism 310, the Z-direction table 311, the X-direction rails 312, and the X-direction table 313 constituting the table 303 are made of non-magnetic materials such as a wood, a resin, a ceramic, and non-magnetic metal.

In the electromagnetic shield device 302, the position measurement device 305 is provided on the +Z direction side of the first opening 302b. The position measurement device 305 is a device used to position the subject 306 or measure a surface shape. The subject 306 mounted on the table 303 passes through the first opening 302b. The subject 306 passes near the position measurement device 305, and thus the position measurement device 305 can easily irradiate the subject 306 with light beams.

The magnetic sensor 304 is provided inside the electromagnetic shield device 302. The magnetic sensor 304 is a sensor which detects a magnetic field generated from the heart of the subject 306. The magnetic sensor 304 is fixed to the electromagnetic shield device 302. The location where the living body magnetic field measurement apparatus 301 is disposed is adjusted to a state in which no magnetic field is substantially present by the electromagnetic shield device 302. Therefore, the magnetic sensor 304 can measure a magnetic field generated from the heart without being influenced by noise. The magnetic sensor 304 detects an intensity component of a magnetic field in a first direction 304a which is the same direction as the Z direction.

The first direction 304a and the second direction 309a are directions orthogonal to each other. The first direction 304a and the third direction 313d are directions orthogonal to each other. The second direction 309a and the third direction 313d are also directions orthogonal to each other. The table 303 moves the subject 306 in the second direction 309a and the third direction 313d orthogonal to each other. The table 303 is moved in an orthogonal coordinate system, and can thus easily control a position of the table 303. A direction in which the electromagnetic shield device 302 extends is the second direction 309a.

A controller 318 is provided at a location separated from the first opening 302b. The controller 318 outputs an electric signal so as to control the living body magnetic field measurement apparatus 301. Specifically, the controller 318 controls the electromagnetic shield device 302, the table 303, the magnetic sensor 304, and the position measurement device 305. A magnetic field or a residual magnetic field is generated due to the electric signal of the controller 318, and becomes noise when detected by the magnetic sensor 304. Since the controller 318 is present at the location separated from the first opening 302b, the magnetic field or the residual magnetic field generated from the controller 318 hardly reaches the magnetic sensor 304. As a result, the magnetic sensor 304 can perform measurement with less noise.

The controller 318 is provided with a display device 321 and an input device 322. The display device 321 is a liquid crystal display (LCD) or an organic light emitting diode (OLED). A measurement situation, a measurement result, and the like are displayed on the display device 321. The input device 322 is constituted of a keyboard, a rotary knob, or the like. An operator operates the input device 322 so as to input various instructions such as a measurement starting instruction or a measurement condition to the living body magnetic field measurement apparatus 301.

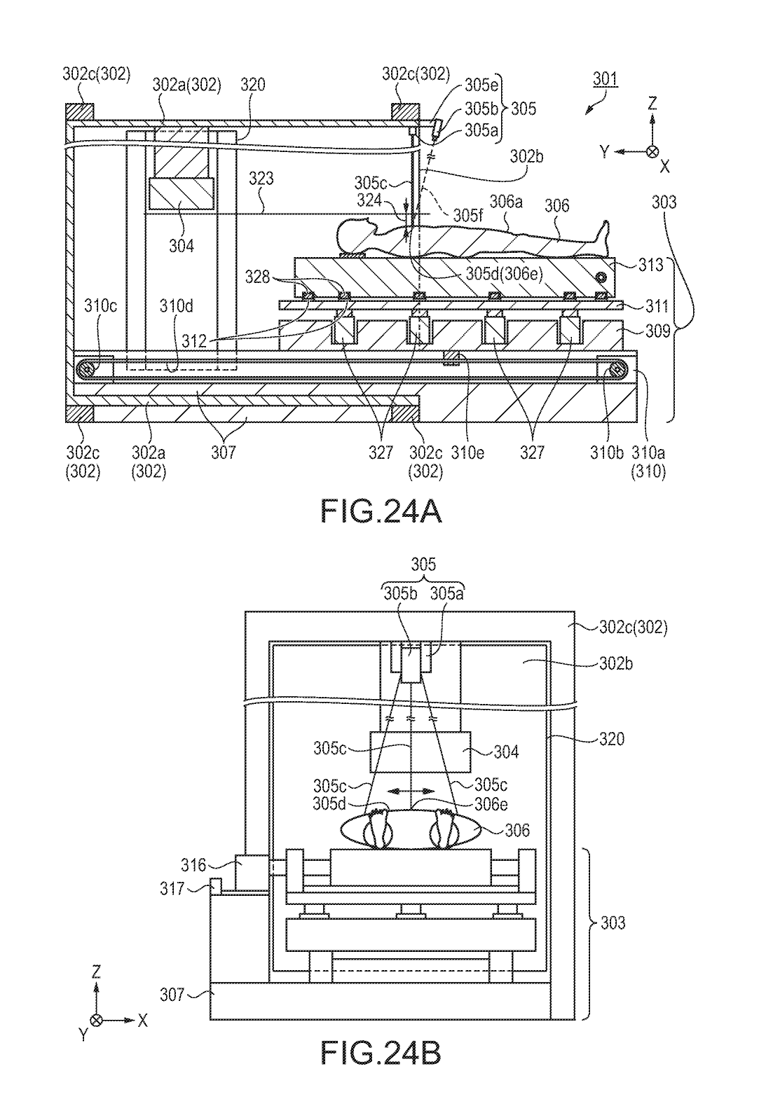

FIG. 24A is a schematic sectional view for explaining a structure of the shape measurement device, and is a view taken along the side surface of the electromagnetic shield device 302. FIG. 24B is a schematic sectional view for explaining a structure of the shape measurement device, and is a view in which the living body magnetic field measurement apparatus 301 is viewed from the −Y direction side. In FIGS. 24A and 24B, the position measurement device 305 includes a laser scanning unit 305a and an imaging device 305b. The laser scanning unit 305a is provided on a ceiling of the main body 302a in the first opening 302b, and emits laser light 305c as light and a light beam in the −Z direction. A front face 306a of the subject 306 is irradiated with the laser light 305c. The laser light 305c is reflected from the front face 306a. The laser scanning unit 305a has a function of performing scanning with the laser light 305c in the X direction, and a function of irradiating a single point without scanning. When the laser scanning unit 305a performs scanning with the laser light 305c, a reflection point 305d at which the laser light 305c is reflected from the front face 306a is linear when viewed from the imaging device 305b. When the laser scanning unit 305a does not perform scanning with the laser light 305c, the reflection point 305d at which the laser light 305c is reflected from the front face 306a is a single point.

When the subject 306 is positioned, the subject 306 is mounted on the table 303 so as to be directed upward. The laser scanning unit 305a irradiates the chest of the subject 306 without scanning with the laser light 305c. The operator drives the Y-direction linear motion mechanism 310 so as to move the Y-direction table 309 in the Y direction. The operator drives the X-direction linear movement mechanism 314 and the X-direction table motor 316 so as to move the X-direction table 313 in the X direction. Positions of the table 303 in the X direction and the Y direction are adjusted so that the laser light 305c is applied to the xiphisternum 306e of the subject 306.

The position measurement device 305 has a function of applying the laser light 305c as guide light, and a function of measuring a position. The function of applying guide light is a function of applying a light beam for guiding a position where the subject 306 is mounted. The function of measuring a position is a function of measuring a shape of the subject by irradiating the subject 306 with a light beam. The position measurement device 305 has the function of applying guide light, and thus the position measurement device 305 applies a light beam for guiding a position where the subject 306 is mounted. Therefore, it is possible to reduce the number of constituent elements compared with a case where the living body magnetic field measurement apparatus 301 separately includes a constituent element applying guide light and a constituent element measuring a position.

The imaging device 305b is provided in the main body 302a via a support portion 305e. The imaging device 305b is obliquely provided with respect to an advancing direction of the laser light 305c. The imaging device 305b images reflected light 305f which is reflected from the front face 306a of the subject 306. In this case, the laser scanning unit 305a, the reflection point 305d, and the imaging device 305b form a triangle. The distance between the laser scanning unit 305a and the imaging device 305b is a known value. An angle formed between the laser light 305c and the reflected light 305f can be detected on the basis of an image captured by the imaging device 305b. Therefore, the position measurement device 305 can measure the distance between the laser scanning unit 305a and the reflection point 305d by using a triangulation method.

A plane which is separated from the surface on the −Z direction side of the magnetic sensor 304 by a predetermined distance in the −Z direction is referred to as a reference plane 323. The reference plane 323 is a plane of a location where a surface for measuring a magnetic field of the subject 306 is disposed. The distance between the surface on the −Z direction side of the magnetic sensor 304 and the reference plane 323 is preferably equal to or more than 2 mm and equal to or less than 10 mm, and is more preferably 5 mm. At this distance, the subject 306 can be made to come close to the magnetic sensor 304 without contact therebetween. In the present embodiment, the distance between the surface on the −Z direction side of the magnetic sensor 304 and the reference plane 323 is, for example, 5 mm. The reference plane 323 is a plane coming into contact with a surface of the chest 306c which is swollen when the subject 306 breathes air. The distance between the laser scanning unit 305a and the reference plane 323 is a known value. The controller 318 calculates a distance 324 between the reference plane 323 and the front face 306a of the subject 306.

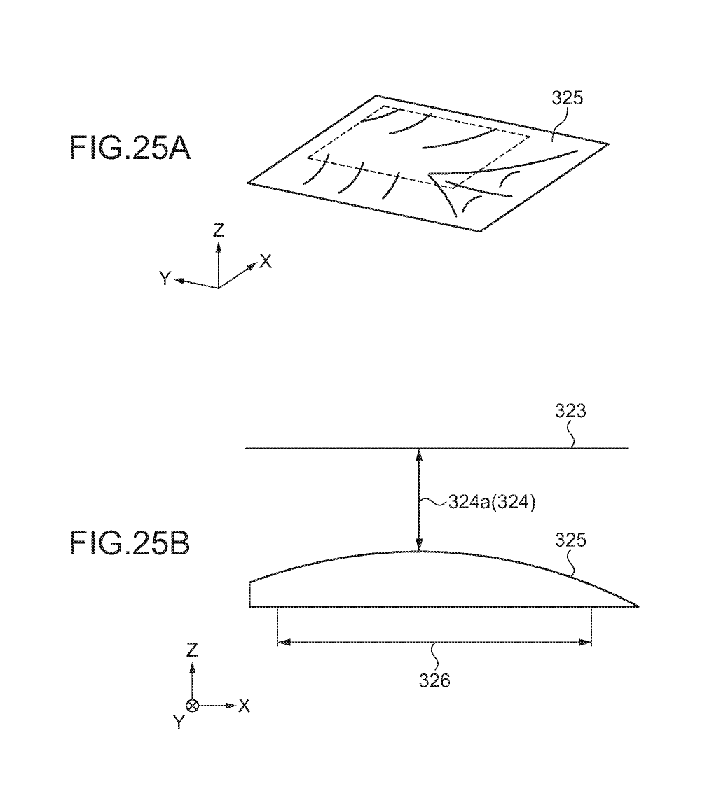

FIG. 25A is a perspective view of a three-dimensional image measured by the position measurement device. As illustrated in FIG. 25A, a three-dimensional image 325 measured by the position measurement device 305 is a stereoscopic image of the chest of the subject 306. The position measurement device 305 measures a shape of the front face 306a of the subject 306 while moving the Y-direction table 309 in the Y direction. The position measurement device 305 measures a stereoscopic image of the chest of the subject 306. The unevenness of the chest is observed on the three-dimensional image 325. FIG. 25B is a schematic side view of a stereoscopic image for explaining measurement in the position measurement device. As illustrated in FIG. 25B, the three-dimensional image 325 includes a location close to the reference plane 323 and a location distant from the reference plane 323. In the three-dimensional image 325, a range measured by the magnetic sensor 304 is set to a magnetic field measurement range 326. FIG. 25B illustrates the magnetic field measurement range 326 in the X direction. The magnetic field measurement range 326 is also set in the Y direction in the same manner. The controller 318 calculates the shortest distance 324a which is a distance 324 to the location closest to the reference plane 323 in the magnetic field measurement range 326 of the three-dimensional image 325.

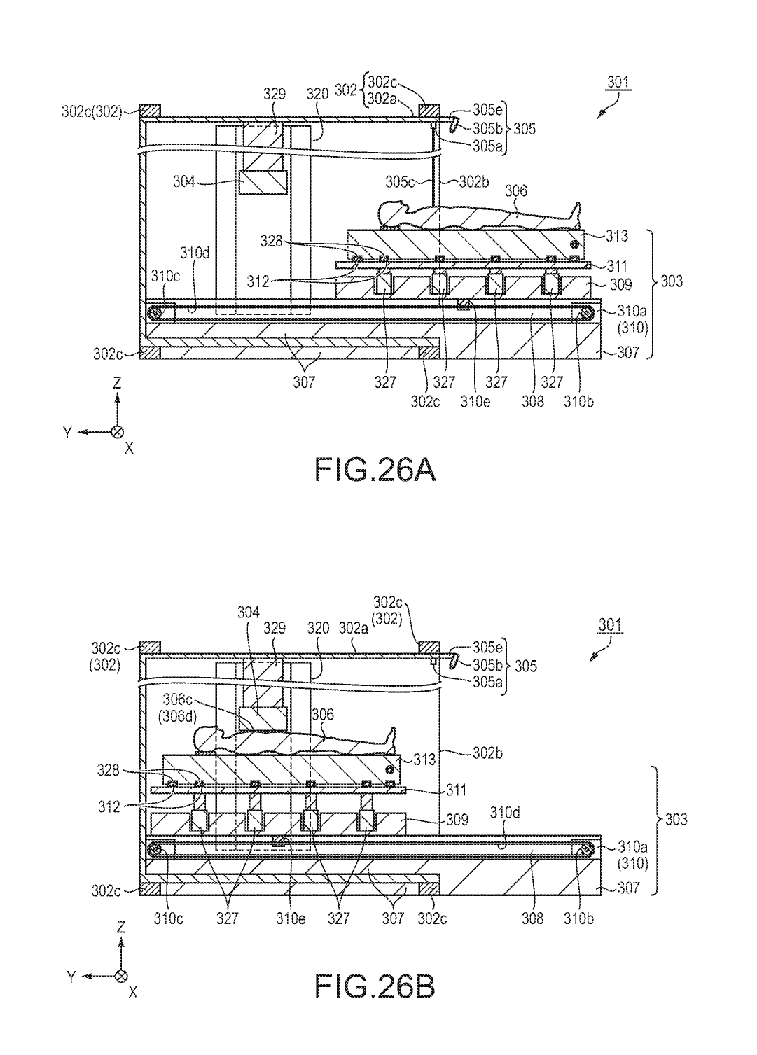

FIGS. 26A and 26B are schematic side sectional views illustrating a structure of the table. FIG. 26A illustrates a state in which the table 303 is being moved in the −Y direction, and FIG. 26B illustrates a state in which the table 303 is moved into the living body magnetic field measurement apparatus 301, and a heart magnetic field of the subject 306 is being measured. As illustrated in FIG. 26A, the pair of first Helmholtz coils 302c is disposed on the foundation 307. A shape of the first Helmholtz coil 302c is a frame shape, and is disposed to surround the main body 302a.

The Y-direction linear motion mechanism 310 includes a motor 310a as a driving source. A first pulley 310b is provided on a rotation shaft of the motor 310a, and a second pulley 310c is rotatably provided at an end on the Y direction side of the Y-direction linear motion mechanism 310. A timing belt 310d is hung on the first pulley 310b and the second pulley 310c. A connection portion 310e is provided on the timing belt 310d, and the connection portion 310e connects the timing belt 310d to the Y-direction table 309. When the motor 310a rotates the first pulley 310b, the connection portion 310e is moved in the Y direction by the torque of the motor 310a. The Y-direction table 309 is moved due to the movement of the connection portion 310e. Therefore, the motor 310a can move the Y-direction table 309 in the Y direction. The motor 310a changes a rotation direction of the first pulley 310b so as to move the Y-direction table 309 in both directions such as +Y direction and the −Y direction.

Materials of the Y-direction rails 308, the second pulley 310c, the timing belt 310d, and the connection portion 310e are non-magnetic materials. The timing belt 310d is made of rubber and resin. The Y-direction rails 308, the second pulley 310c, and the connection portion 310e are made of ceramics. Therefore, a portion of the Y-direction linear motion mechanism 310 entering the electromagnetic shield device 302 is non-magnetic.

Four lifting devices 327 are provided side by side in the Y direction in the Y-direction table 309. Each of the lifting devices 327 has a structure in which three air cylinders are arranged in the X direction. The lifting device 327 expands and contracts the air cylinders so as to lift the Z-direction table 311 in the first direction 304a. Each air cylinder is provided with a length measurement device (not illustrated), and thus the lifting device 327 can detect a movement amount of the Z-direction table 311. The respective air cylinders move the Z-direction table 311 by the same distance, and thus the lifting devices 327 can move the Z-direction table 311 in parallel. Pneumatic equipment such as a compressor and an electromagnetic valve (not illustrated) is provided in the controller 318. The lifting devices 327 are controlled by the controller 318. The Y-direction table 309, the lifting devices 327, and the Z-direction table 311 are made of aluminum. Therefore, the Y-direction table 309, the lifting devices 327, and the Z-direction table 311 are non-magnetic.

The X-direction table 313 is provided with wheels 328 which are in contact with the X-direction rails 312. The wheels 328 are rotated, and thus the X-direction table 313 can be easily moved in the X direction. The X-direction table 313, the X-direction rails 312, and the wheels 328 are made of ceramics which are non-magnetic materials. Therefore, the X-direction table 313, the X-direction rails 312, and the wheels 328 are non-magnetic. A portion of the table 303 entering the electromagnetic shield device 302 is non-magnetic. Consequently, it is possible to prevent magnetization of the table 303 from influencing measurement of a magnetic field.

The magnetic sensor 304 is provided on the ceiling of the main body 302a via a support member 329. A position of the center of the magnetic sensor 304 in the Z direction is the central position between the ceiling of the main body 302a and the bottom of the main body 302a. A position of the center of the magnetic sensor 304 in the X direction is the central position between a wall on the +X direction side of the main body 302a and a wall on the −X direction side thereof. The distance between the center of the magnetic sensor 304 in the Y direction and an end on the −Y direction side of the main body 302a is twice longer than the distance between the center of the magnetic sensor 304 and the wall on the +Y direction side of the main body 302a. When the center of the magnetic sensor 304 is located at this location, it is possible for the magnetic sensor 304 to be hardly influenced by a magnetic field which enters the electromagnetic shield device 302 from the outside thereof.

Second correction coils (second Helmholtz coils 320) having a cube frame shape are provided inside the electromagnetic shield device 302. Specifically, at least three pairs of second correction coils are provided so as to be orthogonal to each other in the X direction, the Y direction, and the Z direction. In the second Helmholtz coil 320 orthogonal to the X direction, a measurement space in which the subject 306 is disposed during measurement and the magnetic sensor 304 are interposed between a pair of coils from the X direction (left/right direction). The second Helmholtz coil 320 orthogonal to the X direction may generate a magnetic field in the X direction and may cancel out an external magnetic field in the X direction so that an X component of a magnetic field of the measurement space and the space in which the magnetic sensor 304 is disposed is reduced to the extent or less of not having an adverse effect on measurement. In the second Helmholtz coil 320 orthogonal to the Y direction, the measurement space and the magnetic sensor 304 are interposed between two pairs of coils (that is, four coils) from the Y direction (front/rear direction). The second Helmholtz coil 320 orthogonal to the Y direction may generate a magnetic field in the Y direction and may cancel out an external magnetic field in the Y direction so that a Y component of a magnetic field of the measurement space and the space in which the magnetic sensor 304 is disposed is reduced to the extent or less of not having an adverse effect on measurement. Since the main body 302a has a tubular shape extending in the front/rear direction, and an entering magnetic field is considerable in the Y direction, two pairs of second Helmholtz coils 320 are provided regarding the Y direction. In the second Helmholtz coil 320 orthogonal to the Z direction, the measurement space and the magnetic sensor 304 are interposed between a pair of coils from the Z direction (upper/lower direction). The second Helmholtz coil 320 orthogonal to the Z direction may generate a magnetic field in the Z direction and may cancel out an external magnetic field in the Z direction so that a Z component of a magnetic field of the measurement space and the space in which the magnetic sensor 304 is disposed is reduced to the extent or less of not having an adverse effect on measurement. The second Helmholtz coil 320 has a square frame shape when viewed from each orthogonal direction side, and is disposed so that the central position of the square frame overlaps the central position of the magnetic sensor 304. A length of the side of the square is not particularly limited, but, in the present embodiment, a length of one side is equal to or more than 75 cm and equal to or less than 85 cm. In FIGS. 26A and 26B, the shape of the second Helmholtz coil 320 is a rectangular shape for better viewing, but is actually a square shape.

The four second Helmholtz coils 320 having the square frame shape are disposed at the same interval in the Y direction. When viewed from the X direction, an outer circumference of the second Helmholtz coil 320 has a square frame shape, and there is a structure in which two coils are disposed inside the square frame shape. The second Helmholtz coil 320 is disposed so that the central position of the square frame shape overlaps the central position of the magnetic sensor 304.

A shape of the second Helmholtz coil 320 viewed from the Z direction is the same as the shape viewed from the X direction. The second Helmholtz coil 320 is disposed so that the central position of the square frame shape overlaps the central position of the magnetic sensor 304. The second Helmholtz coil 320 has such a shape, and thus it is possible to further reduce a disturbance magnetic field in the magnetic sensor 304. Particularly, it is possible to reduce the influence of a magnetic flux which comes from the −Y direction side of the electromagnetic shield device 302.

When the table 303 is located on the −Y direction side of the electromagnetic shield device 302, a half or more of the table 303 protrudes out of the electromagnetic shield device 302. Consequently, the subject 306 is easily mounted on the table 303. When the subject 306 is mounted on the table 303, a height from the floor to the nose of the subject 306 is smaller than a height from the floor to the surface on the −Z direction side of the magnetic sensor 304. Therefore, when the Y-direction table 309 is moved in the Y direction, the subject 306 does not interfere with the magnetic sensor 304.

As illustrated in FIG. 26B, the Y-direction table 309 is moved in the +Y direction, and then the Z-direction table 311 is moved up. A distance by which the Z-direction table 311 is moved up is the shortest distance 324a calculated by the controller 318. A location measured by the magnetic sensor 304 on a surface of the chest 306c of the subject 306 is a measured surface 306d. In this case, the measured surface 306d is located at a location opposing the magnetic sensor 304, and comes close to the magnetic sensor 304. The distance between the measured surface 306d and the magnetic sensor 304 becomes 5 mm. The measured surface 306d is measured by the magnetic sensor 304.

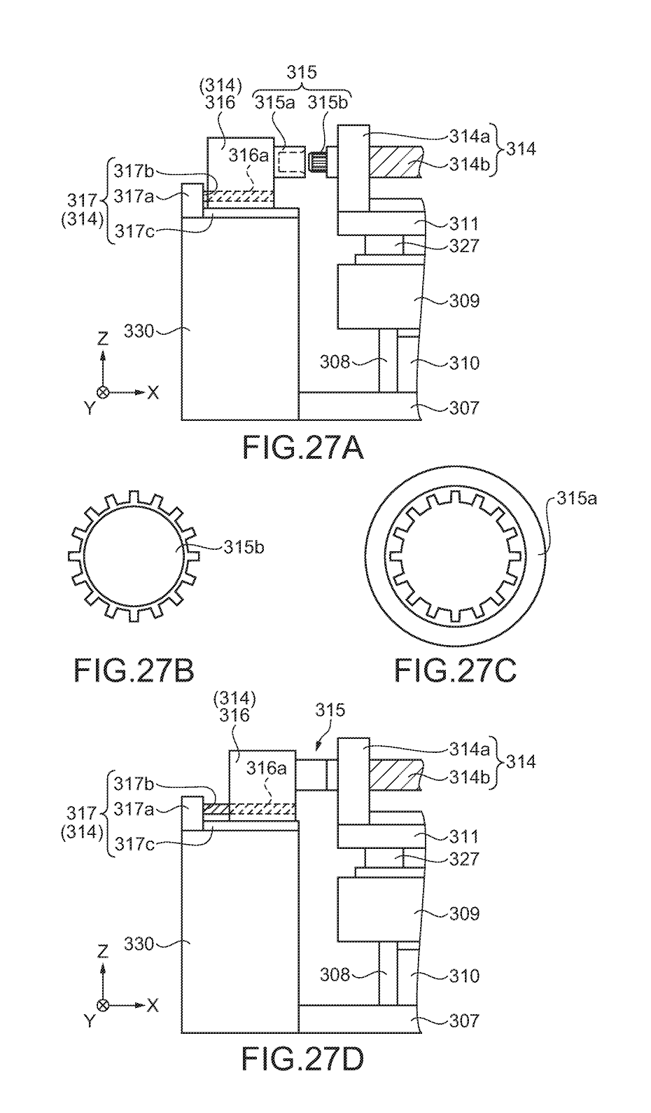

FIG. 27A is a schematic side view illustrating a structure of the attachment/detachment portion, and illustrates a separation state of the attachment/detachment portion 315. As illustrated in FIG. 27A, an attachment/detachment portion installation stand 330 is provided on the −X direction side of the foundation 307. The motor movement portion 317 is provided at an end on the −X direction side on the attachment/detachment portion installation stand 330. The motor movement portion 317 is constituted of a motor 317a, a screw rod 317b, guide rails 317c, and the like. The motor 317a is provided on the −X direction side on the attachment/detachment portion installation stand 330, and the guide rails 317c are provided on the +X direction side of the motor 317a. The guide rails 317c are provided as a pair, and extend in the X direction.

The X-direction table motor 316 is provided on the guide rails 317c, and the X-direction table motor 316 is reciprocally moved along the guide rails 317c. A rotation shaft of the motor 317a is provided with the screw rod 317b extending in the X direction. A penetration hole 316a extending in the X direction is provided in the X-direction table motor 316, and a female screw is formed on the penetration hole 316a. The female screw of the penetration hole 316a is screwed with the screw rod 317b. When the motor 317a rotates the screw rod 317b, the X-direction table motor 316 is moved in the X direction along the guide rails 317c. A rotation shaft of the X-direction table motor 316 is provided with a grooved cylinder 315a. A grooved rod 315b is provided at an end on the −X direction side of the first screw rod 314b. When the X-direction table motor 316 is moved in the X direction, the grooved rod 315b is inserted into the grooved cylinder 315a.

FIG. 27B is a side view of the grooved rod, and is a view in which the grooved rod 315b is viewed from an axial direction. As illustrated in FIG. 27B, grooves are provided on an outer circumference of the grooved rod 315b in an axial direction. FIG. 27C is a side view of the grooved cylinder, and is a view in which the grooved cylinder 315a is viewed from an axial direction. As illustrated in FIG. 27C, grooves extending in the axial direction are provided on an inner diameter of the grooved cylinder 315a. An outer circumference shape of the grooved rod 315b is substantially the same as an inner circumference shape of the grooved cylinder 315a. When the grooved rod 315b is inserted into the grooved cylinder 315a, the grooves of the grooved cylinder 315a mesh with the grooves of the grooved rod 315b. Consequently, the torque applied to the grooved cylinder 315a is transmitted to the grooved rod 315b.

FIG. 27D is a schematic side view illustrating a structure of the attachment/detachment portion, and illustrates a connection state of the attachment/detachment portion 315. In FIG. 27D, the motor movement portion 317 moves the X-direction table motor 316 in the X direction, and thus the grooved rod 315b is inserted into the grooved cylinder 315a. When the X-direction table motor 316 rotates the rotation shaft, the grooved rod 315b is rotated due to rotation of the grooved cylinder 315a. Therefore, when the X-direction table motor 316 rotates the rotation shaft, the first screw rod 314b coupled to the grooved rod 315b is rotated. The X-direction table motor 316 moves the X-direction table 313 in the X direction.

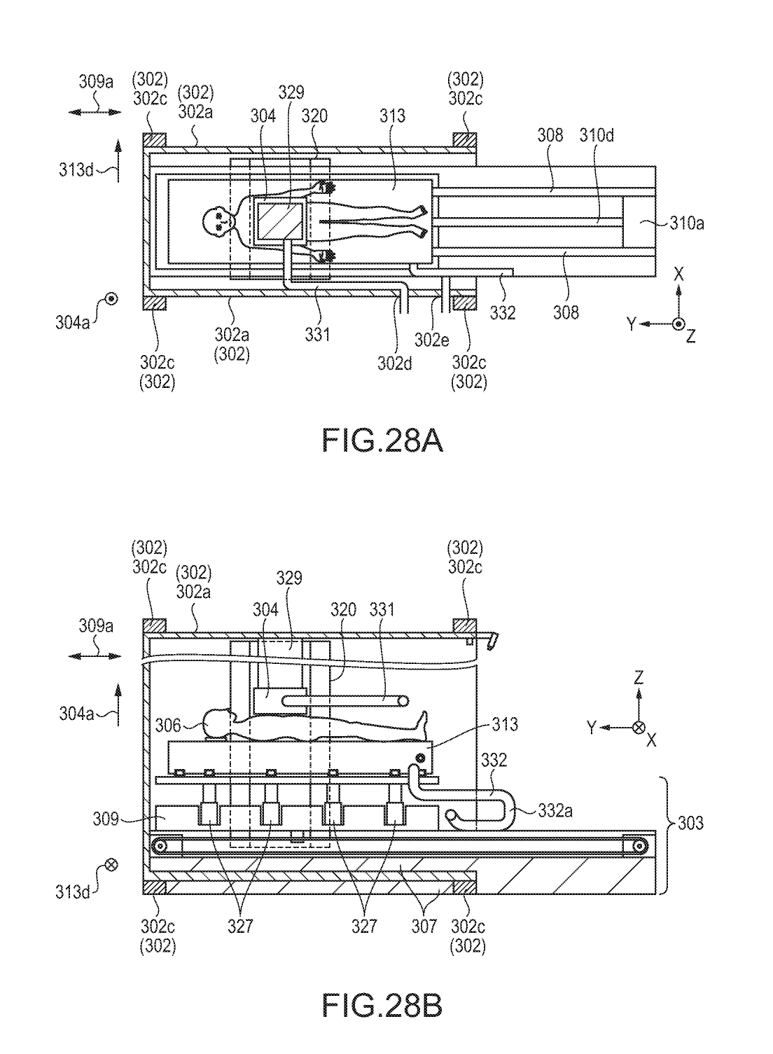

FIG. 28A is a top sectional view for explaining a configuration of a tube, and is a view in which the living body magnetic field measurement apparatus 301 is cut on an XY plane crossing a support member 329. FIG. 28B is a side sectional view for explaining a configuration of the tube, and is a view in which the living body magnetic field measurement apparatus 301 is cut on a YZ plane along the wall on the −X direction side of the electromagnetic shield device 302.

The living body magnetic field measurement apparatus 301 is provided with a first tube 331 as a tube and a second tube 332 as a tube. A wire through which electricity for driving the magnetic sensor 304 flows is provided in the first tube 331. The second tube 332 includes a tube through which air for driving the lifting devices 327 flows.

A second opening 302d and a third opening 302e are provided on a side surface of the −X direction side of the main body 302a. The first tube 331 is disposed through the second opening 302d and allows the inside and the outside of the electromagnetic shield device 302 to communicate with each other. In the second opening 302d, the first tube 331 extends in a third direction 313d orthogonal to the first direction 304a. In the second opening 302d, a direction of a magnetic vector passing through the first tube 331 is orthogonal to the first direction 304a. Therefore, the magnetic vector entering the electromagnetic shield device 302 through the first tube 331 hardly influences the magnetic sensor 304.

Similarly, the second tube 332 is disposed through the third opening 302e and causes the inside and the outside of the electromagnetic shield device 302 to communicate with each other. In the third opening 302e, the second tube 332 extends in the third direction 313d orthogonal to the first direction 304a. In the third opening 302e, a direction of a magnetic vector entering the electromagnetic shield device 302 through the second tube 332 is orthogonal to the first direction 304a. Therefore, the magnetic vector passing through the second tube 332 hardly influences the magnetic sensor 304. As a result, the living body magnetic field measurement apparatus 301 can perform measurement with less noise.

The electromagnetic shield device 302 extends in the second direction 309a. The second direction 309a is a direction orthogonal to the first direction 304a. The first tube 331 extends in the second direction 309a along the main body 302a. Therefore, arrangement is obtained in which the first tube 331 is easily provided. The first tube 331 extends in the second direction 309a, and the second direction 309a is orthogonal to the first direction 304a. Therefore, a magnetic vector passing through the first tube 331 hardly influences the magnetic sensor 304. As a result, the living body magnetic field measurement apparatus 301 can perform measurement with less noise.

The second tube 332 has a structure of tending to be bent, and the second tube 332 is bent twice at a bent portion 332a on the −Y direction side. When the Y-direction table 309 is moved in the Y direction, the bent portion 332a also is moved in the Y direction. Consequently, the second tube 332 can be moved with high durability without being twisted.

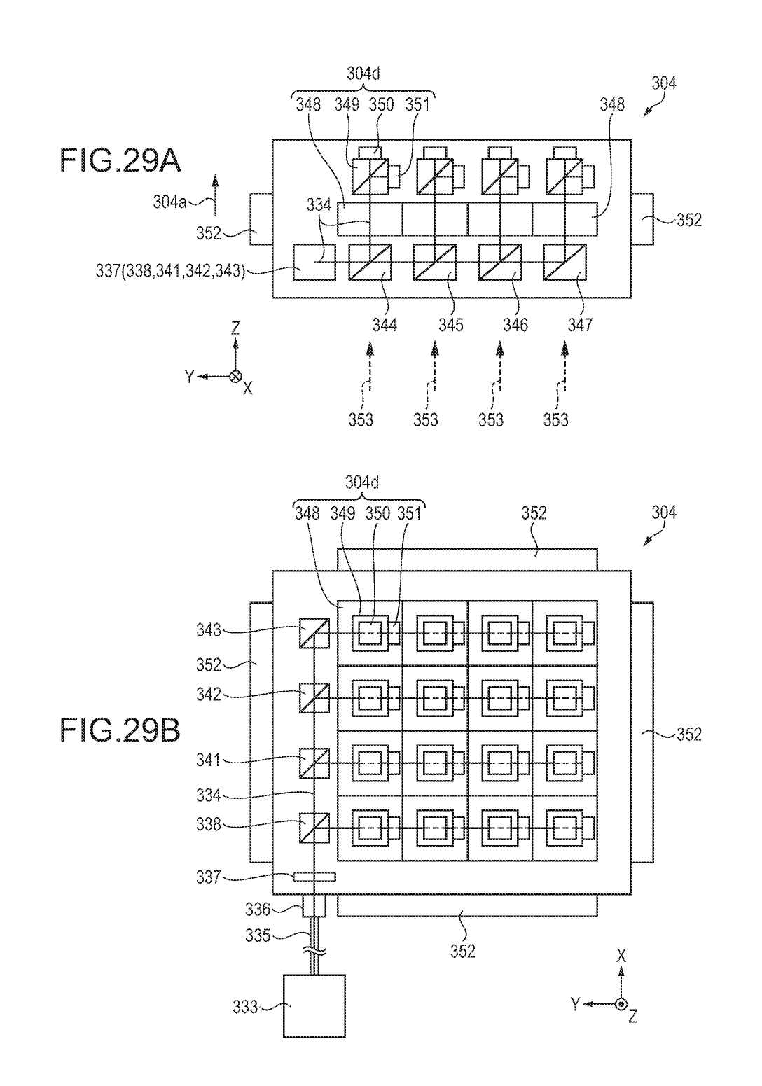

FIG. 29A is a schematic side view illustrating a structure of the magnetic sensor, and FIG. 29B is a schematic plan view illustrating a structure of the magnetic sensor. As illustrated in FIGS. 29A and 29B, laser light 334 is supplied from a laser light source 333 to the magnetic sensor 304. The laser light source 333 is provided in the controller 318, and the laser light 334 is supplied to the magnetic sensor 304 through an optical fiber 335 provided in the first tube 331. The magnetic sensor 304 is coupled to the optical fiber 335 via an optical connector 336.

The laser light source 333 outputs the laser light 334 with a wavelength corresponding to an absorption line of cesium. A wavelength of the laser light 334 is not particularly limited, but is set to a wavelength of 894 nm corresponding to the D1-line, in the present embodiment. The laser light source 333 is a tunable laser device, and the laser light 334 output from the laser light source 333 is continuous light with a predetermined light amount.

The laser light 334 supplied via the optical connector 336 travels in the +X direction and is applied to a polarization plate 337. The laser light 334 having passed through the polarization plate 337 is linearly polarized. The laser light 334 is sequentially applied to a first half mirror 338, a second half mirror 341, a third half mirror 342, and a first reflection mirror 343. Some of the laser light 334 is reflected by the first half mirror 338, the second half mirror 341, and the third half mirror 342 so as to travel in the −Y direction. The other light 334 is transmitted therethrough so as to travel in the +X direction. The first reflection mirror 343 reflects the entire incident laser light 334 in the −Y direction. An optical path of the laser light 334 is divided into four optical paths by the first half mirror 338, the second half mirror 341, the third half mirror 342, and the first reflection mirror 343. Reflectance of each mirror is set so that light intensities of the laser light beams 334 on the respective optical paths are the same as each other.

Next, the laser light 334 is sequentially applied to a fourth half mirror 344, a fifth half mirror 345, a sixth half mirror 346, and a second reflection mirror 347. Some of the laser light 334 is reflected by the fourth half mirror 344, the fifth half mirror 345, and the sixth half mirror 346 so as to travel in the +Z direction. The other light 334 is transmitted therethrough so as to travel in the −Y direction. The second reflection mirror 347 reflects the entire incident laser light 334 in the +Z direction. A single optical path of the laser light 334 is divided into four optical paths by the fourth half mirror 344, the fifth half mirror 345, the sixth half mirror 346, and the second reflection mirror 347. Reflectance of each mirror is set so that light intensities of the laser light beams 334 on the respective optical paths are the same as each other. Therefore, the optical path of the laser light 334 is divided into the sixteen optical paths. In addition, reflectance of each mirror is set so that light intensities of the laser light beams 334 on the respective optical paths are the same as each other.

Gas cells 348 are provided on the respective optical paths of the laser light 334 on the +Z direction side of the fourth half mirror 344, the fifth half mirror 345, the sixth half mirror 346, and the second reflection mirror 347. The number of gas cells 348 is 16 in four rows and four columns. The laser light beams 334 reflected by the fourth half mirror 344, the fifth half mirror 345, the sixth half mirror 346, and the second reflection mirror 347 pass through the gas cells 348. The gas cell 348 is a box having a cavity therein, and an alkali metal gas is enclosed in the cavity. The alkali metal is not particularly limited, and potassium, rubidium, or cesium may be used. In the present embodiment, for example, cesium is used as the alkali metal.

A polarization separator 349 is provided on the +Z direction side of each gas cell 348. The polarization separator 349 is an element which separates the incident laser light 334 into two polarization components of the laser light 334, which are orthogonal to each other. As the polarization separator 349, for example, a Wollaston prism or a polarized beam splitter may be used.

A first photodetector 350 is provided on the +Z direction side of the polarization separator 349, and a second photodetector 351 is provided on the −Y direction side of the polarization separator 349. The laser light 334 having passed through the polarization separator 349 is applied to the first photodetector 350, and the laser light 334 reflected by the polarization separator 349 is applied to the second photodetector 351. The first photodetector 350 and the second photodetector 351 output signals corresponding to an amount of incident laser light 334 to the controller 318. If the first photodetector 350 and the second photodetector 351 generate magnetic fields, this may influence measurement, and thus it is preferable that the first photodetector 350 and the second photodetector 351 are made of a non-magnetic material. The magnetic sensor 304 includes heaters 352 which are provided on both sides in the X direction and both sides in the Y direction. Each of the heaters 352 preferably has a structure in which a magnetic field is not generated, and may employ, for example, a heater of a type of performing heating by causing steam or hot air to pass through a flow passage. Instead of using a heater, the gas cell 348 may be inductively heated by using a high frequency voltage.

The magnetic sensor 304 is disposed on the +Z direction side of the subject 306. A magnetic vector 353 generated from the subject 306 enters the magnetic sensor 304 from the −Z direction side. The magnetic vector 353 passes through the fourth half mirror 344 to the second reflection mirror 347, and then passes through the gas cell 348. The magnetic vector 353 passes through the polarization separator 349, and comes out of the magnetic sensor 304.

The magnetic sensor 304 is a sensor which is called an optical pumping type magnetic sensor or an optical pumping atom magnetic sensor. Cesium in the gas cell 348 is heated and is brought into a gaseous state. The cesium gas is irradiated with the linearly polarized laser light 334, and thus cesium atoms are excited so that orientations of magnetic moments can be aligned. When the magnetic vector 353 passes through the gas cell 348 in this state, the magnetic moments of the cesium atoms precess due to a magnetic field of the magnetic vector 353. This precession is referred to as Larmore precession. The magnitude of the Larmore precession has a positive correlation with the strength of the magnetic vector 353. In the Larmore precession, a polarization plane of the laser light 334 is rotated. The magnitude of the Larmore precession has a positive correlation with a change amount of a rotation angle of the polarization plane of the laser light 334. Therefore, the strength of the magnetic vector 353 has a positive correlation with the change amount of a rotation angel of the polarization plane of the laser light 334. The magnetic sensor 304 has high sensitivity for a component of the magnetic vector 353 in the first direction 304a, and has low sensitivity for a component thereof orthogonal to the first direction 304a.

The polarization separator 349 separates the laser light 334 into two components of linearly polarized light which are orthogonal to each other. The first photodetector 350 and the second photodetector 351 respectively detect the strengths of the two components of linearly polarized light orthogonal to each other. Consequently, the first photodetector 350 and the second photodetector 351 can detect a rotation angle of a polarization plane of the laser light 334. The magnetic sensor 304 can detect the strength of the magnetic vector 353 on the basis of a change of the rotation angle of the polarization plane of the laser light 334. An element constituted of the gas cell 348, the polarization separator 349, the first photodetector 350, and the second photodetector 351 is referred to as a sensor element 304d. In the present embodiment, sixteen sensor elements 304d of four rows and four columns are disposed in the magnetic sensor 304. The number and arrangement of the sensor elements 304d in the magnetic sensor 304 are not particularly limited. The sensor elements 304d may be disposed in three or less rows or five or more rows. Similarly, the sensor elements 304d may be disposed in three or less columns or five or more columns. The larger the number of sensor elements 304d, the higher the spatial resolution.

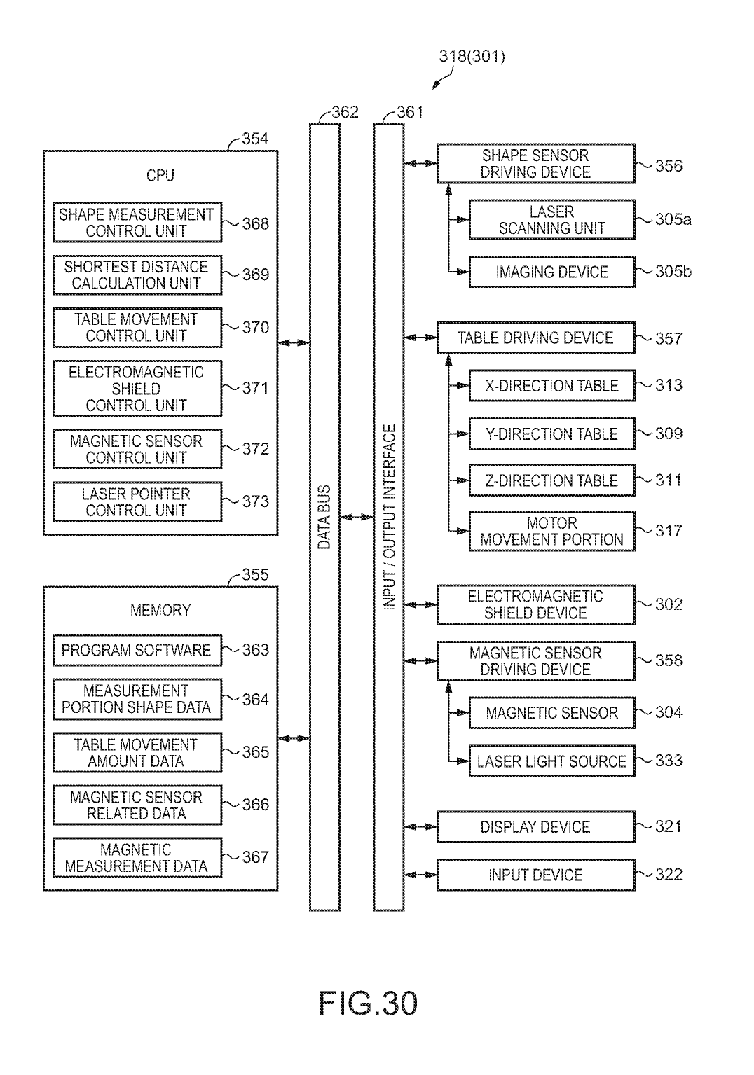

FIG. 30 is an electrical control block diagram of the controller. As illustrated in FIG. 30, the living body magnetic field measurement apparatus 301 includes the controller 318 controlling an operation of the living body magnetic field measurement apparatus 301. The controller 318 includes a central processing unit (CPU) 354 which performs various calculation processes as a processor, and a memory 355 which stores various information. A shape sensor driving device 356, a table driving device 357, the electromagnetic shield device 302, a magnetic sensor driving device 358, the display device 321, and the input device 322 are coupled to the CPU 354 via an input/output interface 361 and a data bus 362.

The shape sensor driving device 356 is a device which drives the laser scanning unit 305a and the imaging device 305b. The shape sensor driving device 356 drives the laser scanning unit 305a to emit the laser light 305c toward the subject 306. The shape sensor driving device 356 performs scanning with the laser light 305c in the horizontal direction. The shape sensor driving device 356 drives the imaging device 305b to capture an image of the reflection point 305d. In addition, the shape sensor driving device 356 irradiates a single location without scanning with the laser light 305c. The irradiated reflection point 305d is a guiding mark indicating a location where the subject 306 is positioned.

The table driving device 357 is a device which drives the X-direction table 313, the Y-direction table 309, the Z-direction table 311, and the motor movement portion 317. The table driving device 357 receives an instruction signal for moving a position of the X-direction table 313 from the CPU 354. The X-direction table 313 can be moved only when the Y-direction table 309 is located at a predetermined position. For this reason, first, the Y-direction table 309 is moved to the predetermined position. The table driving device 357 detects a position of the Y-direction table 309. The Y-direction table 309 includes a length measurement device detecting a position thereof, and the length measurement device detects a position of the Y-direction table 309. The table driving device 357 moves the Y-direction table 309, and thus the Y-direction table 309 is moved to a location where the grooved rod 315b opposes the grooved cylinder 315a.

Next, the table driving device 357 drives the motor movement portion 317 so that the grooved cylinder 315a is combined with the grooved rod 315b. Successively, the table driving device 357 detects a position of the X-direction table 313. The X-direction table 313 includes a length measurement device detecting a position thereof, and the length measurement device detects a position of the X-direction table 313. A difference between a position to which the X-direction table 313 is scheduled to be moved and the present position of the X-direction table 313 is calculated. The table driving device 357 drives the X-direction table motor 316 to move the X-direction table 313 to the position to which the X-direction table 313 is scheduled to be moved. Consequently, the table driving device 357 can move the X-direction table 313 to the location for which an instruction is given. Successively, the table driving device 357 drives the motor movement portion 317 to separate the grooved cylinder 315a from the grooved rod 315b.

Similarly, the table driving device 357 receives an instruction signal for moving a position of the Y-direction table 309 from the CPU 354. The table driving device 357 detects a position of the Y-direction table 309. A difference between a position to which the Y-direction table 309 is scheduled to be moved and the present position of the Y-direction table 309 is calculated. The table driving device 357 drives the motor 310a to move the Y-direction table 309 to the position to which the Y-direction table 309 is scheduled to be moved. Consequently, the table driving device 357 can move the Y-direction table 309 between the position inside the electromagnetic shield device 302 and the position outside the electromagnetic shield device 302. In a case where the position measurement device 305 measures the chest 306c of the subject 306, the Y-direction table 309 is moved at a constant speed.

Similarly, the table driving device 357 receives an instruction signal for moving a position of the Z-direction table 311 from the CPU 354. Each of the lifting devices 327 lifting the Z-direction table 311 includes a length measurement device detecting a position of the Z-direction table 311, and the table driving device 357 detects a position of the Z-direction table 311. A difference between a position to which the Z-direction table 311 is scheduled to be moved and the present position of the Z-direction table 311 is calculated. The lifting device 327 is an air cylinder, and the table driving device 357 is provided with pneumatic equipment such as a compressor or an electromagnetic valve driving the lifting device 327. The table driving device 357 controls an amount of air supplied to the lifting device 327 so as to move the Z-direction table 311 to the position to which the Z-direction table 311 is scheduled to be moved.

The electromagnetic shield device 302 includes the first Helmholtz coil 302c and a sensor detecting an internal magnetic field. The electromagnetic shield device 302 reduces an internal magnetic field of the main body 302a by driving the first Helmholtz coil 302c in response to an instruction from the CPU 354.

The magnetic sensor driving device 358 is a device driving the magnetic sensor 304 and the laser light source 333. The magnetic sensor 304 is provided with the first photodetector 350, the second photodetector 351, and the heater 352. The magnetic sensor driving device 358 drives the laser light source 333, the heater 352, the first photodetector 350, and the second photodetector 351. The magnetic sensor driving device 358 drives the laser light source 333 to supply the laser light 334 to the magnetic sensor 304. The magnetic sensor driving device 358 drives the heater 352 so that the magnetic sensor 304 is maintained at a predetermined temperature. The magnetic sensor driving device 358 converts electric signals output from the first photodetector 350 and the second photodetector 351 into digital signals which are then output to the CPU 354.

The display device 321 displays predetermined information in response to an instruction from the CPU 354. The operator operates the input device 322 on the basis of the display content and inputs the instruction content. The instruction content is transmitted to the CPU 354.

The memory 355 is a concept including a semiconductor memory such as a RAM or a ROM, a hard disk, and an external storage device such as a DVD-ROM. In terms of a function, a storage region for storing program software 363 in which control procedures of an operation of the living body magnetic field measurement apparatus 301 are described, or a storage region for storing measurement portion shape data 364 which is data obtained by measuring a stereoscopic shape of the magnetic field measurement range 326 of the subject 306 is set. In addition, a storage region for storing table movement amount data 365 which is data regarding movement amounts of the Y-direction table 309, and the Z-direction table 311 is set.

Further, a storage region for storing magnetic sensor related data 366 which is data such as parameters used to drive the magnetic sensor 304 is set in the memory 355. Furthermore, a storage region for storing magnetic measurement data 367 which is data measured by the magnetic sensor 304 is set in the memory 355. Moreover, a storage region functioning as a work area for the CPU 354, a temporary file, or the like, and other various storage regions are set.

The CPU 354 controls measurement of a magnetic field generated from the heart of the subject 306 according to the program software 363 stored in the memory 355. As a specific function realizing unit, the CPU 354 includes a shape measurement control unit 368 which is a position measurement unit. The shape measurement control unit 368 controls measurement of a stereoscopic shape of the magnetic field measurement range 326 of the subject 306 by driving the position measurement device 305 and the Y-direction table 309. The CPU 354 includes a shortest distance calculation unit 369. The shortest distance calculation unit 369 calculates the shortest distance 324a by using a measurement result of the stereoscopic shape of the subject 306.

The CPU 354 includes a table movement control unit 370. The table movement control unit 370 controls movement and stoppage positions of the X-direction table 313, and the Y-direction table 309, the Z-direction table 311. The CPU 354 includes an electromagnetic shield control unit 371. The electromagnetic shield control unit 371 performs control for minimizing a magnetic field around the magnetic sensor 304 by driving the electromagnetic shield device 302.

The CPU 354 includes a magnetic sensor control unit 372. The magnetic sensor control unit 372 performs control for causing the magnetic sensor driving device 358 to drive the magnetic sensor 304 to detect the strength of the magnetic vector 353. The CPU 354 includes a laser pointer control unit 373. The laser pointer control unit 373 performs control for driving the laser scanning unit 305a to apply the laser light 305c to only a single point of a predetermined location.

In the present embodiment, the above-described respective functions of the living body magnetic field measurement apparatus 301 are realized in the program software by using the CPU 354, but, in a case where the above-described respective functions can be realized by a stand-alone electronic circuit (hardware) without using the CPU 354, such an electronic circuit may be used.

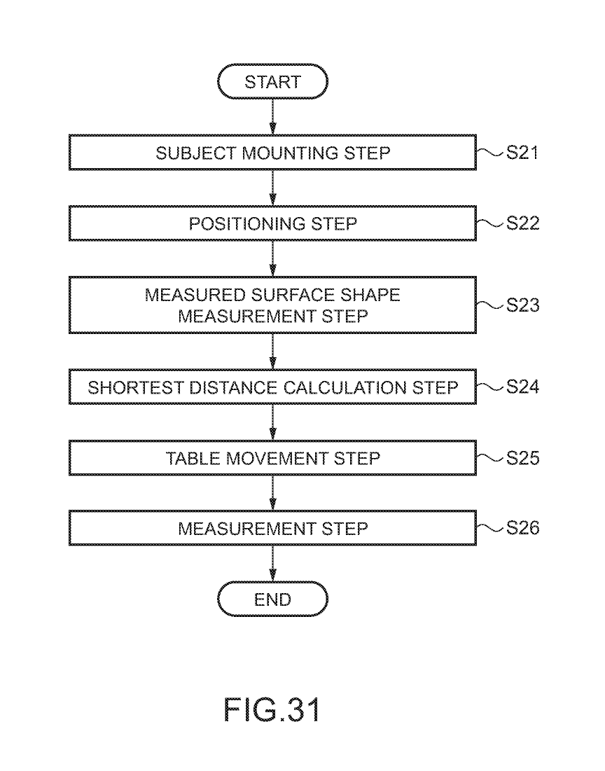

Next, a description will be made of a magnetic field measurement method using the above-described living body magnetic field measurement apparatus 301 with reference to FIGS. 31 to 33C. FIG. 31 is a flowchart illustrating a living body magnetic field measurement method. In the flowchart illustrated in FIG. 31, step S21 is a subject mounting step. In this step, the subject 306 is mounted on the X-direction table 313. Next, the flow proceeds to step S22. Step S22 is a positioning step. In this step, the laser scanning unit 305a irradiates one location of the chest 306c with the laser light 305c. In this step, the operator operates the input device 322 so that the X-direction table 313 and the Y-direction table 309 are moved, and thus the xiphisternum 306e of the subject 306 is irradiated with the reflection point 305d. Next, the flow proceeds to step S23.

Step S23 corresponds to a measured surface shape measurement step. In this step, the shape measurement control unit 368 drives the Y-direction table 309 and the position measurement device 305 to measure a stereoscopic shape of the measured surface 306d of the subject 306. Next, the flow proceeds to step S24. Step S24 is a shortest distance calculation step. In this step, the shortest distance calculation unit 369 calculates the shortest distance 324a by using data regarding the measured stereoscopic shape. Next, the flow proceeds to step S25.

Step S25 is a table movement step. In this step, the table movement control unit 370 moves the table 303 so that the chest 306c of the subject 306 is moved to a location opposing the magnetic sensor 304. The measured surface 306d of the subject 306 comes close to the magnetic sensor 304. Next, the flow proceeds to step S26. Step S26 is a measurement step. In this step, the magnetic sensor control unit 372 causes the magnetic sensor driving device 358 to drive the magnetic sensor 304. The magnetic sensor 304 detects a magnetic field coming out of the chest 306c of the subject 306. Through the above steps, the process of measuring a magnetic field of the subject 306 is finished.

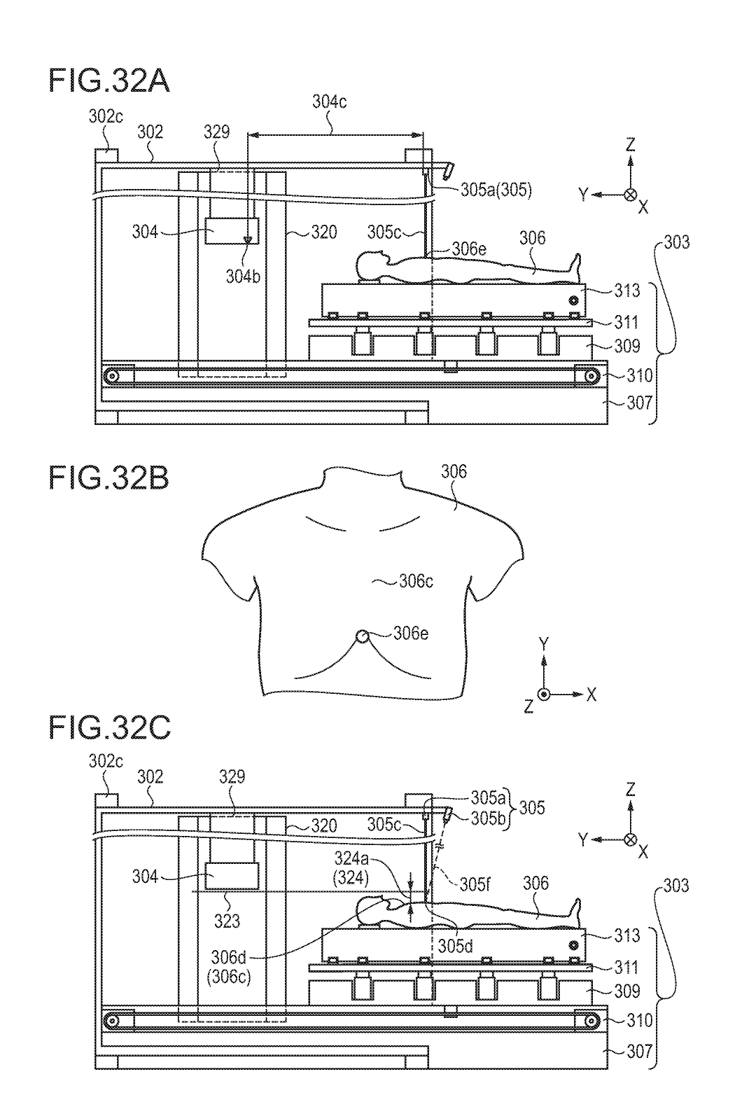

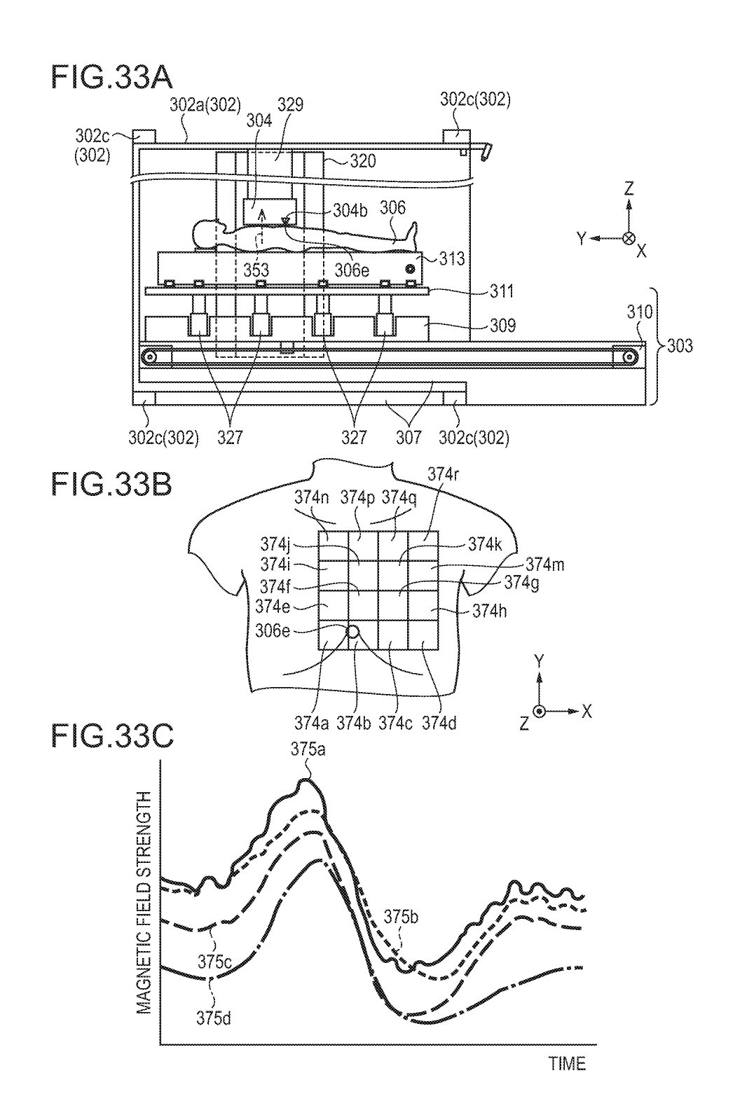

Next, with reference to FIGS. 32A to 33C, the living body magnetic field measurement method will be described in more detail so as to correspond to the steps illustrated in FIG. 31. FIGS. 32A to 33C are schematic diagrams for explaining the living body magnetic field measurement method. FIG. 32A is a diagram corresponding to the subject mounting step of step S21. As illustrated in FIG. 32A, in step S21, the subject 306 is mounted on the X-direction table 313. A half or more of the X-direction table 313 protrudes out of the electromagnetic shield device 302. The Z-direction table 311 is located at a low position, and thus the subject 306 easily moves onto the X-direction table 313.

FIGS. 32A and 32B are diagrams corresponding to the positioning step of step S22. As illustrated in FIG. 32A, in step S22, the operator operates the input device 322 so as to input an instruction for starting positioning. The laser pointer control unit 373 outputs an instruction signal for applying the laser light 305c, to the shape sensor driving device 356. The shape sensor driving device 356 receives the instruction signal so as to drive the laser scanning unit 305a. The laser scanning unit 305a performs irradiation with the laser light 305c in the −Z direction. The laser light 305c is applied to a single point located in the −Z direction from the laser scanning unit 305a.

As illustrated in FIG. 32B, the xiphisternum 306e is present on the −Y direction side of the chest 306c in the subject 306. The xiphisternum 306e is a protrusion which protrudes at the lower end of sternum, and is present at a part called the pit of the stomach at which the rib bows join together. Referring to FIG. 32A again, the operator operates the input device 322 so as to input an instruction for moving the X-direction table 313 in the X direction. The table movement control unit 370 outputs a signal for moving the X-direction table 313, to the table driving device 357. The table driving device 357 drives the motor movement portion 317 to move the X-direction table motor 316 in the +X direction. Consequently, the grooved cylinder 315a is connected to the grooved rod 315b.

Next, the table driving device 357 rotates the X-direction table motor 316 to move the X-direction table 313 in the X direction. The X-direction table 313 is moved in tracking of an instruction which is input by the operator via the input device 322. The operator causes the Y direction side of the xiphisternum 306e to be irradiated with the laser light 305c.

Successively, the operator operates the input device 322 so as to input an instruction for moving the Y-direction table 309. The table movement control unit 370 outputs a signal for moving the Y-direction table 309, to the table driving device 357. The table driving device 357 drives the motor movement portion 317 to move the X-direction table motor 316 in the −X direction. Consequently, the grooved cylinder 315a is separated from the grooved rod 315b.

Next, the table driving device 357 rotates the motor 310a to move the Y-direction table 309 in the Y direction. The Y-direction table 309 is moved in tracking of an instruction which is input by the operator via the input device 322. The operator causes the xiphisternum 306e to be irradiated with the laser light 305c. Thereafter, the operator operates the input device 322 so as to input information indicating that positioning of the subject 306 has been finished.

A reference point 304b for checking a measurement point is set in the magnetic sensor 304. A position of the reference point 304b in the X direction is the same as the position in the X direction where the laser light 305c is applied in step S22. A distance in the Y direction between the position of the reference point 304b and a position through which the laser light 305c passes is set to a predetermined reference distance 304c.

FIG. 32C is a diagram corresponding to the measured surface shape measurement step of step S23 and the shortest distance calculation step of step S24. In step S23, the operator causes the subject 306 to take a normal breath. The subject 306 may take a deep breath so as to control his or her breathing. The operator operates the input device 322 so as to input an instruction for starting measurement of a stereoscopic shape of the measured surface 306d. The shape measurement control unit 368 receives the instruction for starting measurement, and outputs an instruction signal for applying the laser light 305c, to the shape sensor driving device 356. As illustrated in FIG. 32C, the laser scanning unit 305a irradiates the measured surface 306d with the laser light 305c, and reciprocally moves the reflection point 305d in the X direction. The imaging device 305b receives the reflected light 305f. Since the reflection point 305d is reciprocally moved on the measured surface 306d, the imaging device 305b captures an image in which the reflection point 305d forms a line. The shape sensor driving device 356 calculates a distance from the laser scanning unit 305a to the reflection point 305d by using the image data and a triangulation method, and outputs the calculated distance to the memory 355. The memory 355 stores data regarding the distance from the laser scanning unit 305a to the reflection point 305d as a part of the measurement portion shape data 364.

The shape measurement control unit 368 outputs an instruction signal for moving the Y-direction table 309 to the table driving device 357 in cooperation with the table movement control unit 370. A movement range of the Y-direction table 309 is the same as the magnetic field measurement range 326. The table driving device 357 moves the Y-direction table 309 in the −Y direction and then moves the Y-direction table 309 in the +Y direction at a predetermined speed. The table driving device 357 outputs data indicating a position of the Y-direction table 309 in the Y direction to the memory 355. Consequently, the measurement portion shape data 364 of the memory 355 accumulates data regarding the distance between the laser scanning unit 305a and the reflection point 305d in the magnetic field measurement range 326. When the position measurement device 305 completes the measurement in the magnetic field measurement range 326, the table movement control unit 370 outputs an instruction signal for moving the Y-direction table 309 to the table driving device 357 so that the xiphisternum 306e is located at the location opposing the laser scanning unit 305a. The table driving device 357 receives the instruction signal and moves the Y-direction table 309. The operator gives a message that the subject 306 may take a deep breath.

In step S24, the shortest distance calculation unit 369 calculates the distance 324 between the reference plane 323 and the measured surface 306d within the magnetic field measurement range 326. The distance 324 is calculated by subtracting a predetermined value from the distance between the laser scanning unit 305a and the reflection point 305d measured by the position measurement device 305. Next, the shortest distance calculation unit 369 calculates the shortest distance 324a which is the shortest distance among the distances 324 calculated by the shortest distance calculation unit 369.

FIG. 33A is a diagram corresponding to the table movement step of step S25 and the measurement step of step S26. As illustrated in FIG. 33A, in step S25, the table movement control unit 370 outputs an instruction signal for moving the Y-direction table 309, to the table driving device 357. The table driving device 357 receives the instruction signal so as to move the Y-direction table 309 by the reference distance 304c in the +Y direction. Next, the table movement control unit 370 outputs an instruction signal for moving up the Z-direction table 311, to the table driving device 357. The table driving device 357 receives the instruction signal so as to move up the Z-direction table 311 by the shortest distance 324a in the +Z direction. Consequently, the location on the measured surface 306d which is closest to the magnetic sensor 304 matches the reference plane 323.

As a result, the reference point 304b is located at a location opposing the xiphisternum 306e, and the measured surface 306d is located at a location opposing the magnetic sensor 304. The distance between the surface on the −Z direction side of the magnetic sensor 304 and the measured surface 306d becomes 5 mm. When the subject 306 takes a normal breath, a state occurs in which the surface on the −Z direction side of the magnetic sensor 304 is not in contact with the measured surface 306d. Since the magnetic sensor 304 vibrates when the measured surface 306d is in contact with the magnetic sensor 304, the measurement accuracy is reduced. In the present embodiment, since the measured surface 306d is not in contact with the magnetic sensor 304, the living body magnetic field measurement apparatus 301 can detect a magnetic field of the measured surface 306d with high accuracy.

If the magnetic sensor 304 becomes distant from the measured surface 306d, the strength of a magnetic field detected by the magnetic sensor 304 is in inverse proportion to the square of a distance from the measured surface 306d. Therefore, detection performance of the magnetic sensor 304 is reduced as the magnetic sensor 304 becomes more distant from the measured surface 306d. In the present embodiment, since the measured surface 306d comes close to the magnetic sensor 304 to the extent to which the measured surface 306d is not in contact with the magnetic sensor 304, the living body magnetic field measurement apparatus 301 can detect a magnetic field of the measured surface 306d with high accuracy.

The position measurement device 305 is a device which is operated by electricity, and a magnetic field is formed in a case where the position measurement device 305 is operated. Even in a case where the operation of the position measurement device 305 is stopped, a residual magnetic field is formed. The magnetic sensor 304 is provided at a location separated from the position measurement device 305, and is provided inside the electromagnetic shield device 302. The table 303 is moved from the location where the position measurement device 305 measures the measured surface 306d to the location where the magnetic sensor 304 measures the measured surface 306d. Therefore, even if the position measurement device 305 is separated from the magnetic sensor 304, the measured surface 306d can be made to come close to the magnetic sensor 304. As a result, the magnetic sensor 304 can detect a magnetic field of the measured surface 306d with high accuracy without being influenced by the position measurement device 305.

FIGS. 33A to 33C are diagrams corresponding to the measurement step of step S26. As illustrated in FIG. 33A, in step S26, the magnetic sensor 304 detects the magnetic vector 353 which travels in the first direction 304a from the measured surface 306d of the subject 306. The magnetic sensor control unit 372 outputs an instruction signal for starting measurement to the magnetic sensor driving device 358. The magnetic sensor driving device 358 receives the instruction signal for starting measurement, and drives the laser light source 333 and the heater 352. The laser light source 333 applies the laser light 334. If light emission of the laser light source 333 is stabilized, and the magnetic sensor 304 is stabilized at a predetermined temperature, the measurement is started. The strength of a magnetic field detected by the magnetic sensor 304 is output as an electric signal. The magnetic sensor driving device 358 converts electric signals output from the first photodetector 350 and the second photodetector 351 into electric signals indicating the strength of the magnetic field. The magnetic sensor driving device 358 converts the electric signals indicating the strength of the magnetic field into digital data which is then transmitted to the memory 355 as the magnetic measurement data 367.

In FIG. 33B, a first region 374a to a sixteenth region 374r indicate regions where the respective sensor elements 304d detect the magnetic vector 353. The first region 374a to the sixteenth region 374r are disposed in a lattice form of four rows and four columns. The xiphisternum 306e is disposed in the second region 374b. In this arrangement, the magnetic sensor 304 can detect the magnetic vector 353 generated from the heart of the subject 306 without leakage within the range of the first region 374a to the sixteenth region 374r.

FIG. 33C illustrates an example of change data of a magnetic field detected by the magnetic sensor 304. A longitudinal axis expresses the magnetic field strength, and the strength of an upper part in FIG. 33C is higher than the strength of a lower part therein. A transverse axis expresses a change in time, and time changes from a left part to a right part in FIG. 33C. The strength of the magnetic vector 353 detected by the sensor element 304d is referred to as the magnetic field strength. A first change line 375a indicates a change in the magnetic field strength in the twelfth region 374m, and indicates a change in the magnetic field strength on the upper left side of the heart. The upper left side of the heart represents a position in the X direction and the Y direction. A second change line 375b indicates a change in the magnetic field strength in the fourth region 374d, and indicates a change in the magnetic field strength on the lower left side of the heart. A third change line 375c indicates a change in the magnetic field strength in the second region 374b, and indicates a change in the magnetic field strength on the lower right side of the heart. A fourth change line 375d indicates a change in the magnetic field strength in the tenth region 374j, and indicates a change in the magnetic field strength on the upper right side of the heart. Sixteen magnetic field strength change lines can be obtained from the magnetic sensor 304. In FIG. 33C, for better viewing, four change lines are illustrated.

The first change line 375a has a peak, and then the second change line 375b has a peak. Next, the third change line 375c has a peak, and then the fourth change line 375d has a peak. As mentioned above, the peaks of the magnetic field strength move around the heart. When the heart does not normally operate, waveforms of the first change line 375a to the fourth change line 375d are deformed. Therefore, the operator can diagnose heart diseases of the subject 306 by observing waveforms of the first change line 375a to the fourth change line 375d.

After the measurement of a magnetic field is completed, the Z-direction table 311 is moved down, and the Y-direction table 309 is moved in the −Y direction. The subject 306 leaves the table 303, and thus the process of measuring a magnetic field from the heart of the subject 306 is finished.

As described above, according to the present embodiment, the following effects are achieved.

(1) According to the present embodiment, the living body magnetic field measurement apparatus 301 includes the magnetic sensor 304, the position measurement device 305, the table 303, and the controller 318. The magnetic sensor 304 detects a component in the first direction 304a of the magnetic vector 353 coming out of the measured surface 306d of the subject 306. The position measurement device 305 measures a position of the measured surface 306d in the first direction 304a. The subject 306 is mounted on the table 303, and the table 303 moves the subject 306 in the first direction 304a. The controller 318 controls a position of the table 303. The controller 318 controls a distance by which the table 303 is moved on the basis of data regarding a relative position between the magnetic sensor 304 and the measured surface 306d in the first direction 304a, measured by the position measurement device 305. The controller 318 performs control so that the distance between the measured surface 306d and the magnetic sensor 304 is 5 mm.

If the magnetic sensor 304 becomes distant from the measured surface 306d, the strength of a magnetic field detected by the magnetic sensor 304 is in inverse proportion to the square of a distance from the measured surface 306d. Therefore, detection performance of the magnetic sensor 304 is reduced as the magnetic sensor 304 becomes more distant from the measured surface 306d. Since the magnetic sensor 304 vibrates when the measured surface 306d is in contact with the magnetic sensor 304, the measurement accuracy is reduced. In the present embodiment, the measured surface 306d can be made to come close to the magnetic sensor 304 in a range in which the measured surface 306d is not in contact with the magnetic sensor 304. The position measurement device 305 measures a position of the measured surface 306d relative to the magnetic sensor 304, and then the table 303 causes the subject 306 to come close to the magnetic sensor 304. Therefore, even if the position measurement device 305 is separated from the magnetic sensor 304, the subject 306 can be made to come close to the magnetic sensor 304. As a result, the magnetic sensor 304 is hardly influenced by the position measurement device 305, and thus the living body magnetic field measurement apparatus 301 can detect a magnetic field of the measured surface 306d with high accuracy.

(2) According to the present embodiment, the table 303 moves the subject 306 in the second direction 309a and the third direction 313d. The second direction 309a and the third direction 313d are orthogonal to the first direction 304a. The second direction 309a and the third direction 313d intersect each other. Therefore, the table 303 can move the subject 306 in a direction along a plane orthogonal to the first direction 304a. As a result, the table 303 can easily position the subject 306 in the plane direction orthogonal to the first direction 304a.

(3) According to the present embodiment, the second direction 309a is orthogonal to the third direction 313d. The table 303 moves the subject 306 in the second direction 309a and the third direction 313d orthogonal to each other. Therefore, the table 303 can be moved along the orthogonal coordinate system, and thus it is possible to easily control a movement position of the table 303.

(4) According to the present embodiment, the X-direction table motor 316 moves the table 303 in the third direction 313d. The X-direction table motor 316 is provided with the attachment/detachment portion 315 which is located outside the electromagnetic shield device 302, and allows the X-direction table 313 and the X-direction table motor 316 to be attached to or detached from each other. The attachment/detachment portion 315 connects the X-direction table 313 to the X-direction table motor 316, and then the X-direction table 313 is moved in the third direction 313d by using the X-direction table motor 316.

When the X-direction table 313 is not moved in the third direction 313d, the attachment/detachment portion 315 detaches the X-direction table motor 316 from the X-direction table 313. The X-direction table motor 316 can be located outside the electromagnetic shield device 302, and the X-direction table 313 can be moved into the electromagnetic shield device 302. Therefore, it is possible to prevent a magnetic field from the X-direction table motor 316 from influencing the inside of the electromagnetic shield device 302. As a result, the magnetic sensor 304 can perform measurement with less noise.

(5) According to the present embodiment, the position measurement device 305 measures a stereoscopic shape of the measured surface 306d. Therefore, it is possible to detect a position of a most protruding portion of the measured surface 306d. As a result, the measured surface 306d can be made to come close to the magnetic sensor 304 in a range in which the most protruding portion in the measured surface 306d does not come into contact with the magnetic sensor 304.

(6) According to the present embodiment, the position measurement device 305 scans the measured surface 306d with the laser light 305c. A location irradiated with the laser light 305c is measured by using a triangulation method. Therefore, the position measurement device 305 can detect a position of a most protruding portion within a range in which scanning is performed with the laser light 305c.

(7) According to the present embodiment, the position measurement device 305 applies the laser light 305c for guiding positioning of the subject 306. This function is a guide light irradiation function. The position measurement device 305 measures a stereoscopic shape of the measured surface 306d. This function is a position measurement function. The position measurement device 305 has the two functions. Therefore, it is possible to reduce the number of constituent elements compared with a case where the living body magnetic field measurement apparatus 301 separately includes a device having the guide light irradiation function and a device having the position measurement function. As a result, it is possible to manufacture the living body magnetic field measurement apparatus 301 with high productivity.

(8) According to the present embodiment, the position measurement device 305 is provided in the first opening 302b. The subject 306 mounted on the table 303 passes through the first opening 302b. Therefore, the subject 306 passes near the position measurement device 305, and thus the position measurement device 305 can easily irradiate the subject 306 with the laser light 305c.

(9) According to the present embodiment, a portion of the table 303 which is moved into the electromagnetic shield device 302 is non-magnetic. Therefore, it is possible to prevent magnetization of the table 303 from influencing measurement of a magnetic field.

(10) According to the present embodiment, the electromagnetic shield device 302 attenuates an entering magnetic field line. The magnetic sensor 304 and the table 303 are provided in the electromagnetic shield device 302. The electromagnetic shield device 302 is provided with the first opening 302b, and the subject 306 can come in and out of the electromagnetic shield device 302 via the first opening 302b. The controller 318 is located at a location separated from the first opening 302b.

The controller 318 makes an electric signal flow so as to control the table 303. A magnetic field is generated due to the electric signal, and becomes noise when detected by the magnetic sensor 304. In the present embodiment, since the controller 318 is present at the location separated from the first opening 302b, the magnetic field generated from the controller 318 hardly reaches the magnetic sensor 304. As a result, the magnetic sensor 304 can perform measurement with less noise.

(11) According to the present embodiment, the electromagnetic shield device 302 is provided with the first tube 331 and the second tube 332, and the first tube 331 and the second tube 332 extend in a direction orthogonal to the first direction 304a and allow the inside and the outside of the electromagnetic shield device 302 to communicate with each other. Directions of magnetic vectors passing through the first tube 331 and the second tube 332 are orthogonal to the first direction 304a. Therefore, magnetic vectors passing through the first tube 331 and the second tube 332 hardly influence the magnetic sensor 304. As a result, the magnetic sensor 304 can perform measurement with less noise.

(12) According to the present embodiment, the first tube 331 and the second tube 332 extend in the second direction 309a, and the second direction 309a is orthogonal to the first direction 304a. Therefore, magnetic vectors passing through the first tube 331 and the second tube 332 hardly influence the magnetic sensor 304. As a result, the magnetic sensor 304 can perform measurement with less noise. The first tube 331 and the second tube 332 are provided along the electromagnetic shield device 302, and arrangement is obtained in which the first tube 331 the second tube 332 are easily provided.

(13) According to the present embodiment, in the subject mounting step of step S21, the subject 306 is mounted on the table 303. In the measured surface shape measurement step of step S23, a stereoscopic shape of the measured surface 306d of the subject 306 is measured. In the shortest distance calculation step of step S24, the shortest distance 324a to a most protruding portion of the stereoscopic shape is calculated. In the table movement step of step S25, the table 303 is moved so that the most protruding portion comes close to the magnetic sensor 304 with a predetermined gap. In the measurement step of step S26, a distribution of the magnetic vector 353 in the subject 306 is detected.

Therefore, the magnetic sensor 304 comes close to the measured surface 306d in a range in which there is no contact therebetween, and performs measurement. The position measurement device 305 measures a position of the measured surface 306d relative to the magnetic sensor 304, and then the table 303 causes the subject 306 to come close to the magnetic sensor 304. Therefore, even if the position measurement device 305 is separated from the magnetic sensor 304, the subject 306 can be made to come close to the magnetic sensor 304. As a result, the magnetic sensor 304 is hardly influenced by the position measurement device 305, and thus the living body magnetic field measurement apparatus 301 can detect a magnetic field of the measured surface 306d with high accuracy.

(14) According to the present embodiment, the motor 310a of the Y-direction linear motion mechanism 310 is located outside the electromagnetic shield device 302. The motor 310a tends to generate an electromagnetic wave so as to generate a residual magnetic field. Since the motor 310a of the Y-direction linear motion mechanism 310 is located outside the electromagnetic shield device 302, the residual magnetic field of the motor 310a hardly influences the magnetic sensor 304. Therefore, the living body magnetic field measurement apparatus 301 can detect a magnetic field of the measured surface 306d with high accuracy.

The present embodiment is not limited to the above-described embodiments, and various modifications or alterations may be employed by a person skilled in the art within the technical spirit of the invention. Modification examples will be described below.

### Modification Example 1

In the first embodiment, the shape measurement device 5 irradiates the subject 6 with the laser light 5c, and the imaging device 5b images the reflection point 5d. A stereoscopic shape of the measured surface 6d is measured by using an image captured by the imaging device 5b. Other methods may be used to measure a surface shape of the measured surface 6d. For example, measurement may be performed by using an ultrasonic wavelength measurement device or by using interference of laser light. A stereoscopic shape may be measured by lifting a contact type displacement gauge and tracing a surface of the subject 6. An easy measurement method may be selected.

### Modification Example 2

In the first embodiment, an air cylinder is used in the lifting device 24. A hydraulic cylinder may be used in the lifting device 24. Oil is less expandable than air, and thus a movement amount can be controlled with high accuracy. A manual jack may be used in the lifting device 24. The device can be simplified.

### Modification Example 3

In the first embodiment, a length measurement device is provided in the lifting device 24, and a movement amount is feedback. A lifting distance may be controlled by controlling an amount of air supplied to the air cylinder. The number of components can be reduced, and thus it is possible to manufacture the magnetic field measurement apparatus 1 with high productivity.

### Modification Example 4

In the first embodiment, the X-direction linear movement mechanism 14 is of an electric type of being moved by the X-direction table motor 16. The X-direction linear movement mechanism 14 may be of a manual type. It is possible to reduce generation of a magnetic field. The Y-direction linear motion mechanism 10 of the Y-direction table 9 is of an electric type. The Y-direction linear motion mechanism 10 may be of a manual type. It is possible to minimize generation of an electromagnetic wave and thus to prevent the influence of a residual magnetic field.

### Modification Example 5

In the first embodiment, a magnetic field is measured inside the electromagnetic shield device 2. When the magnetic field measurement apparatus 1 is provided in a room in which an electromagnetic wave is blocked, the electromagnetic shield device 2 may be omitted. The number of components can be reduced, and thus it is possible to manufacture the magnetic field measurement apparatus 1 with high productivity.

### Modification Example 6

In the first embodiment, a stereoscopic shape of the measured surface 6d is measured in a state in which the subject 6 takes a normal breath. A stereoscopic shape of the measured surface 6d may be measured in a state in which the lung is swollen. A most protruding portion may be detected, and a position of the most protruding portion may be measured in a state in which the lung is swollen. Even if the subject 6 swells the lung thereof, it is possible to prevent the subject 6 from coming into contact with the magnetic sensor 4.

### Modification Example 7

In the first embodiment, the door 2d is provided at the opening 2b of the electromagnetic shield device 2. In a case where an amount of magnetic fields entering the electromagnetic shield device 2 through the opening 2b is small, the door 2d may be omitted. The number of components can be reduced, and thus it is possible to manufacture the magnetic field measurement apparatus 1 with high productivity.

In a case where the door on the −Y direction side of the electromagnetic shield device 2 is omitted, positions of the magnetic sensor 4 and the second Helmholtz coil 28 are preferably changed. A position of the center of the magnetic sensor 4 in the Y direction is located further in the +Y direction than an intermediate position between the wall on the +Y direction side and the door on the −Y direction side of the main body 2a. A position of the center of the second Helmholtz coil 28 is set to be the same as a position of the center of the magnetic sensor 4. If the center of the magnetic sensor 4 is located at this position, it is possible for the magnetic sensor 4 to be hardly influenced by a magnetic field which enters the electromagnetic shield device 2 from the outside thereof.

### Modification Example 8

In the first embodiment, the electromagnetic shield device 2 includes the rectangular tubular main body 2a. Therefore, the electromagnetic shield device 2 has a quadrangle frame shape as a sectional shape along a plane orthogonal to the Y direction. The electromagnetic shield device 2 may have a frame shape such as a circular shape, a hexagonal shape, or an octagonal shape as a sectional shape along a plane orthogonal to the Y direction. It is possible to further reduce a magnetic field in the magnetic sensor 4.

### Modification Example 9

In the first embodiment, in the table movement step of step S5, the tilting table 18 is tilted with the X direction as an axis. Next, the tilting table 18 is tilted with the Y direction as an axis. These operations may be performed in a reverse procedure. That is, the tilting table 18 may be tilted with the Y direction as an axis, and then the tilting table 18 may be tilted with the X direction as an axis. The operations may be performed in an easily checked procedure. The first tilting portion 26a to the third tilting portion 26c may be simultaneously expanded so that the tilting table 18 is tilted. It is possible to reduce tilting time.

### Modification Example 10

In the first embodiment, in the table movement step of step S5, the tilting table 18 is tilted, and then the Y-direction table 9 is moved. Next, the Z-direction table 11 is moved up. These operations may be performed in a reverse procedure. That is, the Y-direction table 9 is moved in the Y direction, and then the tilting table 18 is tilted. Next, the Z-direction table 11 may be moved up.

### Modification Example 11

In the first embodiment, the table 3 is provided with the lifting device 24 and the tilting device 26. The lifting device 24 and the tilting device 26 may be integrated into a single device. This device may perform lifting and tilting of the tilting table 18. The number of constituent elements can be reduced, and thus it is possible to manufacture the magnetic field measurement apparatus 1 with high productivity.

### Modification Example 12

In the first embodiment, the tilting table 18 is supported by three legs such as the first tilting portion 26a, the second tilting portion 26b, and the third tilting portion 26c. The tilting table 18 may be supported by four or more legs. A load applied to each leg can be reduced.

### Modification Example 13

In the first embodiment, the magnetic sensor 4 detects the magnetic vector 50 which travels in the first direction 4a. The magnetic sensor 4 may detect the magnetic vectors 50 with components in the second direction 9a and the third direction 13d in addition to the first direction 4a. It is possible to more finely detect a motion of the heart 6g.

### Modification Example 14

In the first embodiment, the heater 49 heats the magnetic sensor 4. The heater 49 employs a method in which heating is performed by making steam or hot air pass through a flow passage. Heating may be performed by using a heating wire. In this case, heating is performed before a magnetic field is measured, and heating using the heating wire is stopped when a magnetic field is measured. Consequently, the magnetic sensor 4 can be heated without a magnetic field influencing the magnetic sensor 4. Preferably, a member with large heat capacity is provided in the magnetic sensor 4 so that the temperature of the magnetic sensor 4 hardly decreases.

### Modification Example 15

In the second embodiment, the contour measurement section 102 irradiates the subject 109 with the laser light 113c and the laser light 117c, and the imaging device 113b and the imaging device 117b capture images of the reflection point 113d and the reflection point 117d. A surface shape of the subject 109 is measured by using the images captured by the imaging device 113b and the imaging device 117b. Other methods may be used to measure a surface shape of the subject 109. For example, measurement may be performed by using an ultrasonic wavelength measurement device or by using interference of laser light. A stereoscopic shape may be measured by lifting a contact type displacement gauge and tracing a surface of the subject 109. An easy measurement method may be selected.

### Modification Example 16

In the second embodiment, an air cylinder is used in the lifting device 138. A hydraulic cylinder may be used in the lifting device 138. Similarly, air cylinders are used in the first lifting portion 142 to the tenth lifting portion 151. Hydraulic cylinder may be used in the first lifting portion 142 to the tenth lifting portion 151. Oil is less expandable than air, and thus a movement amount can be controlled with high accuracy. Manual jacks may be used in the lifting device 138, and the first lifting portion 142 to the tenth lifting portion 151. The device can be simplified.

### Modification Example 17

In the second embodiment, length measurement devices are provided in the lifting device 138, and the first lifting portion 142 to the tenth lifting portion 151, and a movement amount is feedback. A lifting distance may be controlled by controlling an amount of air supplied to the air cylinder. The number of components can be reduced, and thus it is possible to manufacture the living body magnetic field measurement apparatus 101 with high productivity.

### Modification Example 18

In the second embodiment, the subject 109 is directly disposed on the contact surface 164. An elastic sheet may be disposed between the contact surface 164 and the subject 109. It is possible to distribute a load of the back face 109b of the subject 109. Consequently, it is possible to reduce pain which the subject 109 may feel due to the table 121 being too hard.

### Modification Example 19

In the second embodiment, the X-direction linear movement mechanism 130 is of a manual type. The X-direction linear movement mechanism 130 may be of an electric type of being moved by a motor. It is possible to move the X-direction table 129 with high operability. The Y-direction linear motion mechanism 126 of the Y-direction table 125 is of an electric type. The Y-direction linear motion mechanism 126 may be of a manual type. It is possible to minimize generation of an electromagnetic wave and thus to prevent the influence of a residual magnetic field.

### Modification Example 20

In the second embodiment, a magnetic field is measured inside the electromagnetic shield device 118. When the magnetic field measurement section 103 is provided in a room in which an electromagnetic wave is blocked, the electromagnetic shield device 118 may be omitted. The number of components can be reduced, and thus it is possible to manufacture the living body magnetic field measurement apparatus 101 with high productivity.

### Modification Example 21

In the second embodiment, widths of the first division surface 152a to the tenth division surface 163a are the same as each other. The widths of the first division surface 152a to the tenth division surface 163a may be different from each other. The widths which easily match a person's shape may be employed. Consequently, it is possible to easily bring the contact surface 164 into contact with the back face 109b of the subject 109.

### Modification Example 22

In the second embodiment, the −Y direction side of the electromagnetic shield device 118 is open without a wall. A door may be provided at the open location on the −Y direction side of the electromagnetic shield device 118. A material of the door is the same as a material of the main body 118a, and is a material blocking a magnetic field. When the Y-direction table 125 enters the electromagnetic shield device 118, the door is closed. Consequently, it is possible to block a magnetic field which travels toward the magnetic sensor 122 from the −Y direction side of the electromagnetic shield device 118. As a result, the magnetic sensor 122 can detect a magnetic field of the subject 109 with higher accuracy without being influenced by disturbance of the magnetic field.

In a case where the door is provided on the −Y direction side of the electromagnetic shield device 118, positions of the magnetic sensor 122 and the second Helmholtz coil 139 are preferably changed. A position of the center of the magnetic sensor 122 in the Y direction is an intermediate position between the wall on the +Y direction side and the door on the −Y direction side of the main body 118a. A position of the center of the second Helmholtz coil 139 is set to be the same as a position of the center of the magnetic sensor 122. If the center of the magnetic sensor 122 is located at this position, it is possible for the magnetic sensor 122 to be hardly influenced by a magnetic field which enters the electromagnetic shield device 118 from the outside thereof.

### Modification Example 23

In the fourth embodiment, the position measurement device 305 irradiates the subject 306 with the laser light 305c, and the imaging device 305b captures an image of the reflection point 305d. A stereoscopic shape of the measured surface 306d is measured by using the image captured by the imaging device 305b. Other methods may be used to measure a stereoscopic surface shape of the measured surface 306d. For example, measurement may be performed by using an ultrasonic wavelength measurement device or by using interference of laser light. A stereoscopic shape may be measured by lifting a contact type displacement gauge and tracing a surface of the subject 306. An easy measurement method may be selected.

### Modification Example 24

In the fourth embodiment, an air cylinder is used in the lifting device 327. A hydraulic cylinder may be used in the lifting device 327. Oil is less expandable than air, and thus a movement amount can be controlled with high accuracy. A manual jack may be used in the lifting device 327. The device can be simplified.

### Modification Example 25

In the fourth embodiment, a length measurement device is provided in the lifting device 327, and a movement amount is feedback. A lifting distance may be controlled by controlling an amount of air supplied to the air cylinder. The number of components can be reduced, and thus it is possible to manufacture the living body magnetic field measurement apparatus 301 with high productivity.

### Modification Example 26

In the fourth embodiment, the X-direction linear movement mechanism 314 is of an electric type of being moved by the X-direction table motor 316. The X-direction linear movement mechanism 314 may be of a manual type. It is possible to reduce generation of a magnetic field. The Y-direction linear motion mechanism 310 of the Y-direction table 309 is of an electric type. The Y-direction linear motion mechanism 310 may be of a manual type. It is possible to minimize generation of an electromagnetic wave and thus to prevent the influence of a residual magnetic field.

### Modification Example 27

In the fourth embodiment, a magnetic field is measured inside the electromagnetic shield device 302. When the living body magnetic field measurement apparatus 301 is provided in a room in which an electromagnetic wave is blocked, the electromagnetic shield device 302 may be omitted. The number of components can be reduced, and thus it is possible to manufacture the living body magnetic field measurement apparatus 301 with high productivity.

### Modification Example 28

In the fourth embodiment, a stereoscopic shape of the measured surface 306d is measured in a state in which the subject 306 takes a normal breath. A stereoscopic shape of the measured surface 306d may be measured in a state in which the lung is swollen. A most protruding portion may be detected, and a position of the most protruding portion may be measured in a state in which the lung is swollen. Even if the subject 306 swells the lung thereof, it is possible to prevent the subject 306 from coming into contact with the magnetic sensor 304.

### Modification Example 29

In the fourth embodiment, the −Y direction side of the electromagnetic shield device 302 is open without a wall. A door may be provided at the open location on the −Y direction side of the electromagnetic shield device 302. A material of the door is the same as a material of the main body 302a, and is a material blocking a magnetic field. When the Y-direction table 309 enters the electromagnetic shield device 302, the door is closed. Consequently, it is possible to block a magnetic field which travels toward the magnetic sensor 304 from the −Y direction side of the electromagnetic shield device 302. As a result, the magnetic sensor 304 can detect a magnetic field of the subject 306 with higher accuracy without being influenced by disturbance of the magnetic field.

In a case where the door is provided on the −Y direction side of the electromagnetic shield device 302, positions of the magnetic sensor 304 and the second Helmholtz coil 320 are preferably changed. A position of the center of the magnetic sensor 304 in the Y direction is an intermediate position between the wall on the +Y direction side and the door on the −Y direction side of the main body 302a. A position of the center of the second Helmholtz coil 320 is set to be the same as a position of the center of the magnetic sensor 304. If the center of the magnetic sensor 304 is located at this position, it is possible for the magnetic sensor 304 to be hardly influenced by a magnetic field which enters the electromagnetic shield device 302 from the outside thereof.

### Modification Example 30

In the fourth embodiment, the electromagnetic shield device 302 includes the rectangular tubular main body 302a. Therefore, the electromagnetic shield device 302 has a quadrangle frame shape as a sectional shape along a plane orthogonal to the Y direction. The electromagnetic shield device 302 may have a frame shape such as a circular shape, a hexagonal shape, or an octagonal shape as a sectional shape along a plane orthogonal to the Y direction. It is possible to further reduce a magnetic field in the magnetic sensor 304.

### Modification Example 31

In the fourth embodiment, the imaging device 305b captures the three-dimensional image 325, and the shortest distance calculation unit 369 calculates the shortest distance 324a. The operator may select a location which is closest to the reference plane 323 in the measured surface 306d, and the location may be measured alone. In other words, the distance 324 may be measured at the location which is highest from the table 303 in the measured surface 306d. The measured value may be used as the shortest distance 324a. It is possible to efficiently measure the shortest distance 324a within a short period of time.

The entire disclosure of Japanese Patent Application No. 2015-116409, filed Jun. 9, 2015 is expressly incorporated by reference herein.

What is claimed is:

1. A magnetic field measurement apparatus comprising:
a detection unit that detects a magnetic field from a subject and that possesses a first surface;
a table on which the subject is mounted;
a measurement unit that measures a surface shape of the subject;
a calculation unit that calculates an average plane of the surface shape; and
a control unit that controls the table so that the first surface is parallel to the average plane.

2. The magnetic field measurement apparatus according to claim 1,
wherein the control unit controls the table so that the distance between the first surface and the subject becomes a predetermined distance.

3. The magnetic field measurement apparatus according to claim 1, further comprising:
a magnetic shield unit that encloses the detection unit and the table, includes an opening through which the subject comes in and out, and attenuates an external magnetic field,
wherein the measurement unit is provided in the opening.

4. The magnetic field measurement apparatus according to claim 1,
wherein the measurement unit scans the subject with a first light.

5. The magnetic field measurement apparatus according to claim 4, further comprising:
a guide light irradiation unit that irradiates a second light for guiding a position where the subject is mounted,
wherein the measurement unit is also used as the guide light irradiation unit.

6. The magnetic field measurement apparatus according to claim 1, wherein
the table includes a plurality of leg portions, and
the control unit controls lengths of the leg portions so as to tilt the subject.

7. The magnetic field measurement apparatus according to claim 3,
wherein a portion of the table is non-magnetic.

8. The magnetic field measurement apparatus according to claim 1,
wherein a location where the detection unit detects a magnetic field is on the heart.

9. A magnetic field measurement method comprising:
mounting a subject on a table;
measuring a surface shape of the subject;
calculating an average plane of the subject;
tilting the table so that a first surface of a detection unit is parallel to the average plane;
causing the subject to come close to the first surface; and
causing the detection unit to detect a magnetic field from the subject.
